SEQUENCE LISTING (446 sequences)

SEQ ID NO: 1 - DNA, 363 bp, synthetic construct
  source 1..363: mol_type = other DNA
gaggtgcagctggtggagtctgggggaggcttggtacagcctggggggtccctgagactctcctgtgcagcctctggattcgcctttagcagctatgccatgacctgggtccgacaggctccagggaaggggctggagtgggtctcagttatcagtggtactggtggtagtacatactacgcagactccgtgaagggccggttcaccatctccagagacaattccaagaacacgctgtatctacaaatgaacagcctgagagccgaggacacggccgtatattactgtgcgaaagggggagcagctcgtagaatggaatacttccagtactggggccagggcaccctggtcaccgtctcctca

SEQ ID NO: 2 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 3 - protein, 8 aa, synthetic construct
GFAFSSYA

SEQ ID NO: 4 - protein, 8 aa, synthetic construct
ISGTGGST

SEQ ID NO: 5 - protein, 14 aa, synthetic construct
AKGGAARRMEYFQY

SEQ ID NO: 6 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaacctccttatctatgctgcatccactttgcaatcaggggtcccatctcgattcagtggcagtggatctgggacagatttcactctcaccatcagcagcctgcagcctgaagatgttgcaacttattactgtcaaaagtataacagtgcccctctcactttcggcggagggaccaaggtggagatcaaa

SEQ ID NO: 7 - protein, 107 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIK

SEQ ID NO: 8 - protein, 6 aa, synthetic construct
QGISNY

SEQ ID NO: 9 - skipped

SEQ ID NO: 10 - protein, 9 aa, synthetic construct
QKYNSAPLT

SEQ ID NO: 11 - DNA, 363 bp, synthetic construct
  source 1..363: mol_type = other DNA
gaggtgcagctggtggagtctgggggaggcttggtacagcctggggggtccctgagactctcctgtgcagcctctggattcacctttaacagctatgccatgacctgggtccgccaggctccagggaaggggctggagtgggtctcatttattggtggtagtactggtaacacatactacgcaggctccgtgaagggccggttcaccatctccagcgacaattccaagaagacgctgtatctgcaaatgaacagcctgagagccgaggacacggccgtatattactgtgcgaaagggggagcagctcgtagaatggaatacttccagcactggggccagggcaccctggtcaccgtctcctca

SEQ ID NO: 12 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 13 - protein, 8 aa, synthetic construct
GFTFNSYA

SEQ ID NO: 14 - protein, 8 aa, synthetic construct
IGGSTGNT

SEQ ID NO: 15 - protein, 14 aa, synthetic construct
AKGGAARRMEYFQH

SEQ ID NO: 16 - DNA, 321 bp, synthetic construct
  source 1..321: mol_type = other DNA
gacatccagatgacccagtctccatcctccctgtctgcatctataggagacagagtcaccatcacttgccgggcgagtcagggcattagcaattatttagcctggtatcaacagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaaccataacagtgtccctctcactttcggcggagggaccaaggtggagatcaaa

SEQ ID NO: 17 - protein, 107 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIK

SEQ ID NO: 18 - protein, 6 aa, synthetic construct
QGISNY

SEQ ID NO: 19 - skipped

SEQ ID NO: 20 - protein, 9 aa, synthetic construct
QNHNSVPLT

SEQ ID NO: 21 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcactacctatggcatgcactgggtccgccaggctccaggcaaggggctggagtgggtggctgttatatggtatgatggaagtaataaatattatggagactccgtgaagggccgattcaccatctccagagacaattccaagaacacactgtatctgcaaatgaacagcctgagagtcgacgacacggctgtttattactgtacgagaacccatggctataccaggtcgtcggacggttttgactactggggccagggaaccctggtcaccgtctcctca

SEQ ID NO: 22 - protein, 122 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 23 - protein, 8 aa, synthetic construct
GFTFTTYG

SEQ ID NO: 24 - protein, 8 aa, synthetic construct
IWYDGSNK

SEQ ID NO: 25 - protein, 15 aa, synthetic construct
TRTHGYTRSSDGFDY

SEQ ID NO: 26 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagaaatgttttaggctggtttcagcagaaaccagggaaagcccctcagcgcctgatctatgctgcatccagtttgcaaagtggggtcccatcaaggttcagcggcagtggatctgggacagaattcactctcacaatcagcagcctacagcctgaagattttgcaacttattactgtctacagcataatttttacccgctcactttcggcggagggaccaaggtggagatcaaa

SEQ ID NO: 27 - protein, 107 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIK

SEQ ID NO: 28 - protein, 6 aa, synthetic construct
QSIRNV

SEQ ID NO: 29 - skipped

SEQ ID NO: 30 - protein, 9 aa, synthetic construct
LQHNFYPLT

SEQ ID NO: 31 - DNA, 369 bp, synthetic construct
  source 1..369: mol_type = other DNA
gaagtgcagctggtggagtctgggggaggcttggtacagcctggcaggtccctgagactctcctgtgcagcctctggattcacctttgatgataaagccatgcactgggtccggcaagttccagggaagggcctggaatggatctcaggtattagttggaatagtggtactataggctatgcggactctgtgaagggccgattcatcatctccagagacaacgccaagaactccctgtatctacaaatgaacagtctgagagctgaggacacggccttgtattactgcgcaaaagatggagataccagtggctggtactggtacggtttggacgtctggggccaagggaccacggtcaccgtctcctca

SEQ ID NO: 32 - protein, 123 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 33 - protein, 8 aa, synthetic construct
GFTFDDKA

SEQ ID NO: 34 - protein, 8 aa, synthetic construct
ISWNSGTI

SEQ ID NO: 35 - protein, 16 aa, synthetic construct
AKDGDTSGWYWYGLDV

SEQ ID NO: 36 - DNA, 321 bp, synthetic construct
  source 1..321: mol_type = other DNA
gaaattgtgttgacacagtctcctgccaccctgtctttgtctccaggggaaagagccaccctctcctgcagggccagtcagagtgttagcagctacttagcctggtaccaacagaaacctggccaggctcccaggctcctcatccatgatgtatccaacagggccactggcatcccagccaggttcagtggcagtgggtctgggacagacttcactctcaccatcagcagtctagagcctgaagattttgtagtttattactgtcagcagcgtagcgactggcccatcaccttcggccaagggacacgactggagattaaa

SEQ ID NO: 37 - protein, 107 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 38 - protein, 6 aa, synthetic construct
QSVSSY

SEQ ID NO: 39 - skipped

SEQ ID NO: 40 - protein, 9 aa, synthetic construct
QQRSDWPIT

SEQ ID NO: 41 - DNA, 360 bp, synthetic construct
  source 1..360: mol_type = other DNA
caggtgcagctggtggagtctgggggaggcgtggtccagcctgggaggtccctgagactctcctgtatagcctctggattcaccttcagtgtctatggcattcactgggtccgccaggctccaggcaaggggctggagtggatggcagtaatatcacatgatggaaatattaaacactatgcagactccgtgaagggccgattcaccatctccagagacaattccaagaacacgctgtatcttcaaattaacagcctgagaactgaggacacggctgtgtattactgtgcgaaagatacctggaactcccttgatacttttgatatctggggccaagggacaatggtcaccgtctcttca

SEQ ID NO: 42 - protein, 120 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 43 - protein, 8 aa, synthetic construct
GFTFSVYG

SEQ ID NO: 44 - protein, 8 aa, synthetic construct
ISHDGNIK

SEQ ID NO: 45 - protein, 13 aa, synthetic construct
AKDTWNSLDTFDI

SEQ ID NO: 46 - DNA, 321 bp, synthetic construct
  source 1..321: mol_type = other DNA
gacatccagttgacccagtctccatcctccctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggagatcaaa

SEQ ID NO: 47 - protein, 107 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 48 - protein, 6 aa, synthetic construct
QGISSY

SEQ ID NO: 49 - skipped

SEQ ID NO: 50 - protein, 9 aa, synthetic construct
QQLNSYPLT

SEQ ID NO: 51 - DNA, 360 bp, synthetic construct
  source 1..360: mol_type = other DNA
caggtcaccttgagggagtctggtcccgcgctggtgaaaccctcacagaccctcacactgacctgcaccttctctggattctcactcaacacttatgggatgtttgtgagctggatccgtcagcctccagggaaggccctagagtggcttgcacacattcattgggatgatgataaatactacagcacatctctgaagaccaggctcaccatctccaaggacacctccaaaaaccaggtggtccttacaatgaccaacatggaccctgtggacacagccacgtattattgtgcacgggggcacaataatttgaactacatcatccactggggccagggaaccctggtcaccgtctcctca

SEQ ID NO: 52 - protein, 120 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 53 - protein, 10 aa, synthetic construct
GFSLNTYGMF

SEQ ID NO: 54 - protein, 7 aa, synthetic construct
IHWDDDK

SEQ ID NO: 55 - protein, 12 aa, synthetic construct
ARGHNNLNYIIH

SEQ ID NO: 56 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccactttacaaagtggggtcccatcaaggttcagcggcagtggatctggcacagatttcactctcaccatcagcagcctgcagcctgaagattttgcaacttattactgtctacaagattacaattacccattcactttcggccctgggaccaaagtggatatcaaa

SEQ ID NO: 57 - protein, 106 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDI

SEQ ID NO: 58 - protein, 6 aa, synthetic construct
QGIRND

SEQ ID NO: 59 - skipped

SEQ ID NO: 60 - protein, 9 aa, synthetic construct
LQDYNYPFT

SEQ ID NO: 61 - DNA, 348 bp, synthetic construct
  source 1..348: mol_type = other DNA
gaggtgcagctggtggagtctgggggaggcttggtacagcctggagggtccctgagactctcctgtgcagtctctggattcatcttcagtagttatgaaatgaactgggtccgccaggctccagggaaggggctggagtgggtttcatacattagtagtagtggtagtaccatattctacgcagactctgtgaagggccgattcaccatctccagagacaacgccaagaactcactgtatctgcaaatgaacagcctgagagccgaggacacggctgtttattactgtgtgtctggagtggtcctttttgatgtctggggccaagggacaatggtcaccgtctcttca

SEQ ID NO: 62 - protein, 116 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 63 - protein, 8 aa, synthetic construct
GFIFSSYE

SEQ ID NO: 64 - protein, 8 aa, synthetic construct
ISSSGSTI

SEQ ID NO: 65 - protein, 9 aa, synthetic construct
VSGVVLFDV

SEQ ID NO: 66 - DNA, 321 bp, synthetic construct
  source 1..321: mol_type = other DNA
gaaatagtgatgacgcagtctccagccaccctgtctgtgtctccgggggaaagagccaccctctcctgcagggccagtcagagtgttagcagcaactttgcctggtaccaacagaaacctggccaggctcccaggctcctcatctatagtgcatcctccagggccactggtatcccagtcaggttcagtggcagtgggtctgggacagagttcactctcaccatcagcagcctgcagtctgaagattttgcagtttattactgtcagcagtataatatctggcctcggacgttcggccaagggaccaaggtggaaatcaaa

SEQ ID NO: 67 - protein, 107 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 68 - protein, 6 aa, synthetic construct
QSVSSN

SEQ ID NO: 69 - skipped

SEQ ID NO: 70 - protein, 9 aa, synthetic construct
QQYNIWPRT

SEQ ID NO: 71 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaactattggatgacctgggtccgccaggctccagggaaggggctggagtgggtggccaacataaaggaagatggaagtgagaaagactatgtggactctgtgaagggccgattcaccatctccagagacaacgccaagaactcactgtatctgcaaatgaacagcctgagaggcgaggacacggctgtgtattactgtgcgagagatggggagcagctcgtcgattactactactactacgttatggacgtctggggccaagggaccacggtcaccgtctcctca

SEQ ID NO: 72 - protein, 125 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 73 - protein, 8 aa, synthetic construct
GFTFSNYW

SEQ ID NO: 74 - protein, 8 aa, synthetic construct
IKEDGSEK

SEQ ID NO: 75 - protein, 18 aa, synthetic construct
ARDGEQLVDYYYYYVMDV

SEQ ID NO: 76 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccgtacacttttggccaggggaccaagctggagatcaaa

SEQ ID NO: 77 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIK

SEQ ID NO: 78 - protein, 6 aa, synthetic construct
QGISSW

SEQ ID NO: 79 - skipped

SEQ ID NO: 80 - protein, 9 aa, synthetic construct
QKANSFPYT

SEQ ID NO: 81 - DNA, 381 bp, synthetic construct
  source 1..381: mol_type = other DNA
gaagtgcagctggtggagtctgggggaggcttggtacagcctggcaggtccctgagactctcctgtgcagcctctggattcacctttgatgactatgccatgcactgggtccggcaagctccagggaagggcctggagtgggtctcaggtattagttggaatagtggttacataggctatgcggactctgtgaagggccgattcaccatctccagagacaacgccgagaactccctacatctgcaaatgaacagtctgagagctgaggacacggccttgtattactgtgcaagagggggatctactctggttcggggagttaagggaggctactacggtatggacgtctggggccaagggaccacggtcaccgtctcctca

SEQ ID NO: 82 - protein, 127 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 83 - protein, 8 aa, synthetic construct
GFTFDDYA

SEQ ID NO: 84 - protein, 8 aa, synthetic construct
ISWNSGYI

SEQ ID NO: 85 - protein, 20 aa, synthetic construct
ARGGSTLVRGVKGGYYGMDV

SEQ ID NO: 86 - DNA, 321 bp, synthetic construct
  source 1..321: mol_type = other DNA
gacatccagatgacccagtctccatcctccctgtctgcatctgtaggagacagagtcaccatcacttgccgggcaagtcagagcataagtagctatttaaattggtatcagcagaaaccaggtaaagcccctaaggtcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattccgctcactttcggcggagggaccaaggtggagatcaaa

SEQ ID NO: 87 - protein, 107 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIK

SEQ ID NO: 88 - protein, 6 aa, synthetic construct
QSISSY

SEQ ID NO: 89 - skipped

SEQ ID NO: 90 - protein, 9 aa, synthetic construct
QQSYSIPLT

SEQ ID NO: 91 - DNA, 351 bp, synthetic construct
  source 1..351: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacatttccaagaacacgctgtatctgcaaatgaacagcctgagagccgaggacacggccgtatattactgtgcgggtcaactggatctcttctttgactactggggccagggaaccctggtcaccgtctcctca

SEQ ID NO: 92 - protein, 117 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 93 - protein, 8 aa, synthetic construct
GFTFSSYG

SEQ ID NO: 94 - protein, 8 aa, synthetic construct
IWYDGSNK

SEQ ID NO: 95 - protein, 10 aa, synthetic construct
AGQLDLFFDY

SEQ ID NO: 96 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattgacaggtatttaaattggtatcggcagaaaccagggaaagcccctaagctcctgatctatactacatccagtttgcaaagtggggtcccatcaaggttcagtggcagtggatctgggacagatttcactctcaccctcagcagtctgcagcctgaagattttgcaacttactactgtcagcagagttacagtcccccgctcactttcggcggagggaccaaggtggagatcaaa

SEQ ID NO: 97 - protein, 107 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIK

SEQ ID NO: 98 - protein, 6 aa, synthetic construct
QSIDRY

SEQ ID NO: 99 - skipped

SEQ ID NO: 100 - protein, 9 aa, synthetic construct
QQSYSPPLT

SEQ ID NO: 101 - DNA, 375 bp, synthetic construct
  source 1..375: mol_type = other DNA
gaggtgcagctggtggagtctgggggaggcttggtccagcctggggggtccctgagactctcctgtacagcctctggattcacctttagtaactattggatgacctgggtccgccaggctccagggaaggggctggagtgggtggccaacataaaggaagatggaagtgagaaagagtatgtggactctgtgaagggccggttcaccatctccagagacaacgccaagaattcactgtatctgcaaatgaacagcctgagaggcgaggacacggctgtatattactgtgcgagagatggggagcagctcgtcgattactattactactacgttatggacgtctggggccaagggaccacggtcaccgtctcctca

SEQ ID NO: 102 - protein, 125 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 103 - protein, 8 aa, synthetic construct
GFTFSNYW

SEQ ID NO: 104 - protein, 8 aa, synthetic construct
IKEDGSEK

SEQ ID NO: 105 - protein, 18 aa, synthetic construct
ARDGEQLVDYYYYYVMDV

SEQ ID NO: 106 - DNA, 321 bp, synthetic construct
  source 1..321: mol_type = other DNA
gacatccagatgacccagtctccatcttccgtgtctgcatctgttggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctgacagtctcccgtacgcttttggccaggggaccaagctggagatcaaa

SEQ ID NO: 107 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIK

SEQ ID NO: 108 - protein, 6 aa, synthetic construct
QGISSW

SEQ ID NO: 109 - skipped

SEQ ID NO: 110 - protein, 9 aa, synthetic construct
QKADSLPYA

SEQ ID NO: 111 - DNA, 363 bp, synthetic construct
  source 1..363: mol_type = other DNA
gaggtgcagctggtggagtctgggggaggcttggttcagcctggggggtccctgagactctcctgtgcaacctctggattcacctttaccagctatgacatgaagtgggtccgccaggctccagggctgggcctggagtgggtctcagctattagtggtagtggtggtaacacatactacgcagactccgtgaagggccggttcaccatctccagagacaattccaggaacacgctgtatctgcaaatgaacagcctgagagccgaggacacggccgtatattactgtacgaggtcccatgacttcggtgccttcgactactttgactactggggccagggaaccctggtcaccgtctcctca

SEQ ID NO: 112 - protein, 121 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 113 - protein, 8 aa, synthetic construct
GFTFTSYD

SEQ ID NO: 114 - protein, 8 aa, synthetic construct
ISGSGGNT

SEQ ID NO: 115 - protein, 14 aa, synthetic construct
TRSHDFGAFDYFDY

SEQ ID NO: 116 - DNA, 321 bp, synthetic construct
  source 1..321: mol_type = other DNA
gacatccagatgacccagtctccatcctccctgtctgcatctgtgggagacagagtcaccatcacttgccgggcaagtcagggcattagagatcattttggctggtatcagcagaaaccagggaaagcccctaagcgcctgatctatgctgcatccagtttgcacagtggggtcccatcaaggttcagcggcagtggatctgggacagaattcactctcacaatcagcagcttgcagcctgaagattttgcaacctattactgtctacagtatgatacttacccgctcactttcggcggagggaccaaggtggagatcaaa

SEQ ID NO: 117 - protein, 107 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 118 - protein, 6 aa, synthetic construct
QGIRDH

SEQ ID NO: 119 - skipped

SEQ ID NO: 120 - protein, 9 aa, synthetic construct
LQYDTYPLT

SEQ ID NO: 121 - DNA, 366 bp, synthetic construct
  source 1..366: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggagtgggtctcaggtattagttggaatagtgctaccagagtctatgcggactctgtgaagggccgattcaccatctccagagacaacgccaagaatttcctgtatctgcaaatgaacagtctgagatctgaggacacggccttgtatcactgtgcaaaagatatggatatctcgctagggtactacggtttggacgtctggggccaagggaccacggtcaccgtctcctca

SEQ ID NO: 122 - protein, 122 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 123 - protein, 8 aa, synthetic construct
GFTFDDYA

SEQ ID NO: 124 - protein, 8 aa, synthetic construct
ISWNSATR

SEQ ID NO: 125 - protein, 15 aa, synthetic construct
AKDMDISLGYYGLDV

SEQ ID NO: 126 - DNA, 324 bp, synthetic construct
  source 1..324: mol_type = other DNA
gaaatagtgatgacgcagtctccagccaccctgtctgtgtctccaggggaaagagccaccctctcctgcagggccagtcagactgttagcagcaacttagcctggtatcagcagaaacctggccaggctcccaggctcctcatctatggttcatcctccagggccactggtatcccagccaggttcagtggcagtgggtctgggacagagttcactctcaccatcagcagcctgcagtctgaagattttgcagtttattactgtcagcagtataataactggcctccctacacttttggccaggggaccaagctggagatcaaa

SEQ ID NO: 127 - protein, 108 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIK

SEQ ID NO: 128 - protein, 6 aa, synthetic construct
QTVSSN

SEQ ID NO: 129 - skipped

SEQ ID NO: 130 - protein, 10 aa, synthetic construct
QQYNNWPPYT

SEQ ID NO: 131 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctca

SEQ ID NO: 132 - protein, 124 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 133 - protein, 10 aa, synthetic construct
GFSLSTSGVG

SEQ ID NO: 134 - protein, 7 aa, synthetic construct
IYWNDHK

SEQ ID NO: 135 - protein, 16 aa, synthetic construct
AHYSGSYSYYYYGLDV

SEQ ID NO: 136 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaa

SEQ ID NO: 137 - protein, 107 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIK

SEQ ID NO: 138 - protein, 6 aa, synthetic construct
QGIASW

SEQ ID NO: 139 - skipped

SEQ ID NO: 140 - protein, 9 aa, synthetic construct
QQANYFPWT

SEQ ID NO: 141 - DNA, 363 bp, synthetic construct
  source 1..363: mol_type = other DNA
gaggtgcagctgttggagtctgggggagccttggtacagcctggggggtccctgagactctcctgtgcagcctctggattcacctttacctcctatgccatgcactgggtccgccaggctccagggaagggtctggagtgggtctcatctattagaggtagtggtggtggcacatactccgcagactccgtgaagggccggttcaccatctccagagacaattccagggacactctatatctgcaaatgaacagtgtgagagccgaggacacggccgtttattactgtgcgaggtcccatgactacggtgccttcgacttctttgactactggggccagggaaccctggtcaccgtctcctca

SEQ ID NO: 142 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 143 - protein, 8 aa, synthetic construct
GFTFTSYA

SEQ ID NO: 144 - protein, 8 aa, synthetic construct
IRGSGGGT

SEQ ID NO: 145 - protein, 14 aa, synthetic construct
ARSHDYGAFDFFDY

SEQ ID NO: 146 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaactgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcggcctgaagattttgcaactttttactgtctacagtataatagttacccgctcactttcggcggagggaccaaggtggagatcaaa

SEQ ID NO: 147 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIK

SEQ ID NO: 148 - protein, 6 aa, synthetic construct
QGIRTD

SEQ ID NO: 149 - skipped

SEQ ID NO: 150 - protein, 9 aa, synthetic construct
LQYNSYPLT

SEQ ID NO: 151 - DNA, 348 bp, synthetic construct
  source 1..348: mol_type = other DNA
caggtgcagctggtggagtctgggggaggcttggtcaagcctggagggtccctgagactctcctgtgcagcctctggattcaccttcagtgactacttcatgagctggatccgccaggctccagggaaggggctggagtgggtttcatacattagtagtactggtagtaccataaattatgcagactctgtgaagggccgattcaccatctccagggacaatgtcaagaattcactgtatctgcaaatgaccagcctgagagtcgaggacacggccgtgtattactgtacgagagataactggaactatgaatactggggccagggaaccctggtcaccgtctcctca

SEQ ID NO: 152 - protein, 116 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 153 - protein, 8 aa, synthetic construct
GFTFSDYF

SEQ ID NO: 154 - protein, 8 aa, synthetic construct
ISSTGSTI

SEQ ID NO: 155 - protein, 9 aa, synthetic construct
TRDNWNYEY

SEQ ID NO: 156 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcatcaacttagcctggtaccagcagaaacctggccaggctcccaggctcctcatctttgttgcatccaccagggccactggtatcccagccaggttcagtggcagtgggtctgggacagagttcactctcaccatcagcagcctgcagtctgaagattttgcaacttattactgtcagcagtatgatatctggccgtacacttttggccaggggaccaagctggagatcaaa

SEQ ID NO: 157 - protein, 107 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIK

SEQ ID NO: 158 - protein, 6 aa, synthetic construct
QSVSIN

SEQ ID NO: 159 - skipped

SEQ ID NO: 160 - protein, 9 aa, synthetic construct
QQYDIWPYT

SEQ ID NO: 161 - DNA, 369 bp, synthetic construct
  source 1..369: mol_type = other DNA
cagctgcagctgcaggagtcgggcccaggactggtgaagccttcggagaccctgtccctcacctgcactgtgtctggtgaatccatcagcagtaatacttactactggggctggatccgccagcccccagggaaggggctggaatggattgggagtatcgattatagtgggaccaccaattataacccgtccctcaagagtcgagtcaccatatccgtagacacgtccaggaatcacttctccctgaggctgaggtctgtgaccgccgcagacacggctgtgtattactgtgcgagagagtggggaaactacggctactattacggtatggacgtttggggccaagggaccacggtcaccgtctcctca

SEQ ID NO: 162 - protein, 123 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 163 - protein, 10 aa, synthetic construct
GESISSNTYY

SEQ ID NO: 164 - protein, 7 aa, synthetic construct
IDYSGTT

SEQ ID NO: 165 - protein, 15 aa, synthetic construct
AREWGNYGYYYGMDV

SEQ ID NO: 166 - DNA, 321 bp, synthetic construct
  source 1..321: mol_type = other DNA
gacatccagatgacccagtctccatcctccctgtctgcatctgtaggagacagagtcaccatcaattgccgggcaagtcagggcattagaaatgatttaggctggtatcagcagaaaccagggaaagcccctaagcgcctgatctatgctgcatccagtttgcaaagtggggtcccattaaggttcagtggcagtggatctgggacagaattcactctcacaatcaacaacctgcagcctgaagattttgcaacttattactgtctatcgcataatagttacccgtggacgttcggccaagggaccaaggtggaaatcaaa

SEQ ID NO: 167 - protein, 107 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIK

SEQ ID NO: 168 - protein, 6 aa, synthetic construct
QGIRND

SEQ ID NO: 169 - skipped

SEQ ID NO: 170 - protein, 9 aa, synthetic construct
LSHNSYPWT

SEQ ID NO: 171 - DNA, 354 bp, synthetic construct
  source 1..354: mol_type = other DNA
caggtgcagctggtgcagtctggggctgaggtgaagaagcctgggtcctcggtgagggtctcctgcaaggcttctagaggcaccttcagcagctatgctatcagctgggtgcgacaggcccctggacaaggccttgagtggatgggagggatcatccccatctttggtacagcaaactacgcacagaagttcctggccagagtcacgattaccgcggacgaatccacgagcacagcctacatggagctgagcagcctgagatctgaggacacggccgtgtattactgtgcgagagagaaggggtggaactactttgactactggggccagggaaccctggtcaccgtctcctca

SEQ ID NO: 172 - protein, 118 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 173 - protein, 8 aa, synthetic construct
RGTFSSYA

SEQ ID NO: 174 - protein, 8 aa, synthetic construct
IIPIFGTA

SEQ ID NO: 175 - protein, 11 aa, synthetic construct
AREKGWNYFDY

SEQ ID NO: 176 - DNA, 321 bp, synthetic construct
  source 1..321: mol_type = other DNA
gacatccagatgacccagtctccaccttccgtgtctgcatctgtaggagacagagtcaccatcacttgtcgggcgagtcagggtattagcagctggttagcctggtatcagcagaaaccagggaaagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctcggacgttcggccaagggaccaaggtggaaatcaaa

SEQ ID NO: 177 - protein, 107 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIK

SEQ ID NO: 178 - protein, 6 aa, synthetic construct
QGISSW

SEQ ID NO: 179 - skipped

SEQ ID NO: 180 - protein, 9 aa, synthetic construct
QQANSFPRT

SEQ ID NO: 181 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattagtagtagtgggactaccatatactacgcagactctgtgaagggccgattcaccatctccagggacaacgccaagaaatcactgtatctggagatgaacagcctcagagccgaggacacggccgtgtactactgtgcgagagaggggtacggtaatgactactattactacggtatagacgtctggggccaagggaccacggtcaccgtctcctca

SEQ ID NO: 182 - protein, 123 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 183 - protein, 8 aa, synthetic construct
GFTFSDYY

SEQ ID NO: 184 - protein, 8 aa, synthetic construct
ISSSGTTI

SEQ ID NO: 185 - protein, 16 aa, synthetic construct
AREGYGNDYYYYGIDV

SEQ ID NO: 186 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatggtaatggatacaactatttgacttggtacctgcagaagccagggcagtctccacagctcctgatctatttgggttctaatcgggcctccggggtccctgacaggttcagtggcagtggatcaggcacagattttacactgaaaataagcagagtggaggctgaggatgttggggtttattactgcatgcaagctctacaaactccgtacacttttggccaggggaccaagctggagatcaaa

SEQ ID NO: 187 - protein, 112 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIK

SEQ ID NO: 188 - protein, 11 aa, synthetic construct
QSLLHGNGYNY

SEQ ID NO: 189 - skipped

SEQ ID NO: 190 - protein, 9 aa, synthetic construct
MQALQTPYT

SEQ ID NO: 191 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagctttggcatgcactgggtccgccaggctccaggcaaggggctggagtgggtgatatttatatcatatgatggaagtgataaatactatgcagactccgtgaagggccgattcgccatctccagagacagttccaagaacacgctatatctgcaaatgaacagcctgagagctgaggacacggctgtgtattactgtgcgaaagaaaacggtattttgactgattcctacggtatggacgtctggggccaagggaccacggtcaccgtctcctca

SEQ ID NO: 192 - protein, 122 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 193 - protein, 8 aa, synthetic construct
GFTFSSFG

SEQ ID NO: 194 - protein, 8 aa, synthetic construct
ISYDGSDK

SEQ ID NO: 195 - protein, 15 aa, synthetic construct
AKENGILTDSYGMDV

SEQ ID NO: 196 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 197 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 198 - protein, 6 aa, synthetic construct
QSISSY

SEQ ID NO: 199 - skipped

SEQ ID NO: 200 - protein, 10 aa, synthetic construct
QQSYSTPPIT

SEQ ID NO: 201 - DNA, 372 bp, synthetic construct
  source 1..372: mol_type = other DNA
caggttcagctggtgcagtctggagctgaggtgaagaagcctggggcctctgtgaaggtctcctgcaaggcttctggttacacctttaccagctatggtatcagctgggtgcgacaggcccctggacaagggcttgagtggatgggatggatcagtgtttaccatggtaacacaaactatgcacagaagttccagggcagagtcaccatgaccacagacacatccacgagcacagcctacatggagctgaggagcctgagatctgacgacacggccgtgtattactgtgcgagagaggggtattacgatttttggagtggttattacccttttgactactggggccagggaaccctggtcaccgtctcctca

SEQ ID NO: 202 - protein, 124 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 203 - protein, 8 aa, synthetic construct
GYTFTSYG

SEQ ID NO: 204 - protein, 8 aa, synthetic construct
ISVYHGNT

SEQ ID NO: 205 - protein, 17 aa, synthetic construct
AREGYYDFWSGYYPFDY

SEQ ID NO: 206 - DNA, 324 bp, synthetic construct; identical to SEQ ID NO: 196
  source 1..324: mol_type = other DNA

SEQ ID NO: 207 - protein, 108 aa, synthetic construct; identical to SEQ ID NO: 197

SEQ ID NO: 208 - protein, 6 aa, synthetic construct
QSISSY

SEQ ID NO: 209 - skipped

SEQ ID NO: 210 - protein, 10 aa, synthetic construct
QQSYSTPPIT

SEQ ID NO: 211 - DNA, 354 bp, synthetic construct
  source 1..354: mol_type = other DNA
gaggtgcagctggtggagtctgggggaggcttgatacaacctggagggtccctgagactctcctgtgaagcctctggattcaccttcagaaattatgaaatgaattgggtccgccaggctccagggaaggggctggagtgggtttcatatattagtagtagtggtaatatgaaagactacgcagagtctgtgaagggccgattcaccatctccagagacaatgtcaagaattcactgcagctgcaaatgaacagcctgagagtcgaggacacggctgtttattactgtgcgagagacgagtttccttacggaatggacgtctggggccaagggaccacggtcaccgtctcctca

SEQ ID NO: 212 - protein, 118 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 213 - protein, 8 aa, synthetic construct
GFTFRNYE

SEQ ID NO: 214 - protein, 8 aa, synthetic construct
ISSSGNMK

SEQ ID NO: 215 - protein, 11 aa, synthetic construct
ARDEFPYGMDV

SEQ ID NO: 216 - DNA, 324 bp, synthetic construct; identical to SEQ ID NO: 196
  source 1..324: mol_type = other DNA

SEQ ID NO: 217 - protein, 108 aa, synthetic construct; identical to SEQ ID NO: 197

SEQ ID NO: 218 - protein, 6 aa, synthetic construct
QSISSY

SEQ ID NO: 219 - skipped

SEQ ID NO: 220 - protein, 10 aa, synthetic construct
QQSYSTPPIT

SEQ ID NO: 221 - DNA, 363 bp, synthetic construct
  source 1..363: mol_type = other DNA
gaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctca

SEQ ID NO: 222 - protein, 121 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 223 - protein, 8 aa, synthetic construct
GFTFDDYA

SEQ ID NO: 224 - protein, 8 aa, synthetic construct
ISWSSGSM

SEQ ID NO: 225 - protein, 14 aa, synthetic construct
AKAREVGDYYGMDV

SEQ ID NO: 226 - DNA, 324 bp, synthetic construct; identical to SEQ ID NO: 196
  source 1..324: mol_type = other DNA

SEQ ID NO: 227 - protein, 108 aa, synthetic construct; identical to SEQ ID NO: 197

SEQ ID NO: 228 - protein, 6 aa, synthetic construct
QSISSY

SEQ ID NO: 229 - skipped

SEQ ID NO: 230 - protein, 10 aa, synthetic construct
QQSYSTPPIT

SEQ ID NO: 231 - DNA, 357 bp, synthetic construct
  source 1..357: mol_type = other DNA
gaagtgcagctggtggagtctgggggaggcttggtacagcctggcaggtccctgacactctcctgtgcagcctctggattcacctttgataattttggcatgcactgggtccggcaaggtccagggaagggcctggaatgggtctcaggtcttacttggaatagtggtgtcataggctatgcggactctgtgaagggccgattcaccatctccagagacaacgccaagaactccctgtatctgcaaatgaacagtctgagacctgaggacacggccttatattactgtgcaaaagatatacggaattacggcccctttgactactggggccagggaaccctggtcaccgtctcctca

SEQ ID NO: 232 - protein, 119 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 233 - protein, 8 aa, synthetic construct
GFTFDNFG

SEQ ID NO: 234 - protein, 8 aa, synthetic construct
LTWNSGVI

SEQ ID NO: 235 - protein, 12 aa, synthetic construct
AKDIRNYGPFDY

SEQ ID NO: 236 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 237 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 238 - protein, 7 aa, synthetic construct
QSVSSSY

SEQ ID NO: 239 - skipped

SEQ ID NO: 240 - protein, 9 aa, synthetic construct
QQYGSSPWT

SEQ ID NO: 241 - DNA, 357 bp, synthetic construct
  source 1..357: mol_type = other DNA
gaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctca

SEQ ID NO: 242 - protein, 119 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 243 - protein, 8 aa, synthetic construct
GFTFNIFE

SEQ ID NO: 244 - protein, 8 aa, synthetic construct
ISSRGTTT

SEQ ID NO: 245 - protein, 12 aa, synthetic construct
ARDYEATIPFDF

SEQ ID NO: 246 - DNA, 324 bp, synthetic construct; identical to SEQ ID NO: 196
  source 1..324: mol_type = other DNA

SEQ ID NO: 247 - protein, 108 aa, synthetic construct; identical to SEQ ID NO: 197

SEQ ID NO: 248 - protein, 6 aa, synthetic construct
QSISSY

SEQ ID NO: 249 - skipped

SEQ ID NO: 250 - protein, 10 aa, synthetic construct
QQSYSTPPIT

SEQ ID NO: 251 - DNA, 363 bp, synthetic construct
  source 1..363: mol_type = other DNA
gaggtgcagctggtggagtctgggggaagtgtggtacggcctggggggtccctgagactctcctgtgaagcctctggattcacctttgatgattatggcatgagctgggtccgccaagatccagggaaggggctggagtgggtctctggtattaattggaatggtgatagaacaaattatgcagactctgtgaagggccgattcatcatttccagagacaacgccaagaactctgtgtatctacaaatgaacagtctgagagcggaggactcggccttgtatcactgtgcgagagatcagggactcggagtggcagctacccttgactactggggccagggaaccctggtcaccgtctcctca

SEQ ID NO: 252 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 253 - protein, 8 aa, synthetic construct
GFTFDDYG

SEQ ID NO: 254 - protein, 8 aa, synthetic construct
INWNGDRT

SEQ ID NO: 255 - protein, 14 aa, synthetic construct
ARDQGLGVAATLDY

SEQ ID NO: 256 - DNA, 324 bp, synthetic construct; identical to SEQ ID NO: 196
  source 1..324: mol_type = other DNA

SEQ ID NO: 257 - protein, 108 aa, synthetic construct; identical to SEQ ID NO: 197

SEQ ID NO: 258 - protein, 6 aa, synthetic construct
QSISSY

SEQ ID NO: 259 - skipped

SEQ ID NO: 260 - protein, 10 aa, synthetic construct
QQSYSTPPIT

SEQ ID NO: 261 - DNA, 384 bp, synthetic construct
  source 1..384: mol_type = other DNA
gaggtgcagctggtggagtctgggggaggcttggtacagcctggagggtccctgagactctcctgtgcagcctctggattcaccgtcagtaattatgaaatgaactgggtccgccaggctccagggaaggggctggagtgggtttcatacattagtagtagtaccagtaacatatactacgcagactctgtgaagggccgattcaccatctccagagacaacgccgagaactcactgtatctgcagatgaacagcctgagagtcgaggacacggctgtttattactgtgtgagagatgggattgtagtagttccagttggtcgtggatactactattacggtttggacgtctggggccaagggaccacggtcaccgtctcctca

SEQ ID NO: 262 - protein, 94 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVY

SEQ ID NO: 263 - protein, 8 aa, synthetic construct
GFTVSNYE

SEQ ID NO: 264 - protein, 8 aa, synthetic construct
ISSSTSNI

SEQ ID NO: 265 - protein, 21 aa, synthetic construct
VRDGIVVVPVGRGYYYYGLDV

SEQ ID NO: 266 - DNA, 324 bp, synthetic construct; identical to SEQ ID NO: 196
  source 1..324: mol_type = other DNA

SEQ ID NO: 267 - protein, 108 aa, synthetic construct; identical to SEQ ID NO: 197

SEQ ID NO: 268 - protein, 6 aa, synthetic construct
QSISSY

SEQ ID NO: 269 - skipped

SEQ ID NO: 270 - protein, 10 aa, synthetic construct
QQSYSTPPIT

SEQ ID NO: 271 - DNA, 378 bp, synthetic construct
  source 1..378: mol_type = other DNA
caggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctca

SEQ ID NO: 272 - protein, 126 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 273 - protein, 8 aa, synthetic construct
GFPFSNYV

SEQ ID NO: 274 - protein, 8 aa, synthetic construct
IFFDGKKN

SEQ ID NO: 275 - protein, 19 aa, synthetic construct
AKIHCPNGVCYKGYYGMDV

SEQ ID NO: 276 - DNA, 324 bp, synthetic construct; identical to SEQ ID NO: 196
  source 1..324: mol_type = other DNA

SEQ ID NO: 277 - protein, 108 aa, synthetic construct; identical to SEQ ID NO: 197

SEQ ID NO: 278 - protein, 6 aa, synthetic construct
QSISSY

SEQ ID NO: 279 - skipped

SEQ ID NO: 280 - protein, 10 aa, synthetic construct
QQSYSTPPIT

SEQ ID NO: 281 - DNA, 375 bp, synthetic construct
  source 1..375: mol_type = other DNA
gaggtgcagctggtggagtctgggggaggcttggtccagcctggggggtccctaagactctcctgtgcagcctctggattcacctttagtaactattggatgaactgggtccgccaggctccagggaagggactggagtgggtggccaatataaaagaagatggaggtaagaaattgtatgtggactctgtgaagggccgattcaccatctccagagacaacgccaagaactcactgtttctgcaaatgaacagcctgagagccgaggacacggctgtgtattattgtgcgagagaagatacaactttggttgtggactactactactacggtatggacgtctggggccaagggaccacggtcaccgtctcctca

SEQ ID NO: 282 - protein, 125 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 283 - protein, 8 aa, synthetic construct
GFTFSNYW

SEQ ID NO: 284 - protein, 8 aa, synthetic construct
IKEDGGKK

SEQ ID NO: 285 - protein, 18 aa, synthetic construct
AREDTTLVVDYYYYGMDV

SEQ ID NO: 286 - DNA, 324 bp, synthetic construct; identical to SEQ ID NO: 196
  source 1..324: mol_type = other DNA

SEQ ID NO: 287 - protein, 108 aa, synthetic construct; identical to SEQ ID NO: 197

SEQ ID NO: 288 - protein, 6 aa, synthetic construct
QSISSY

SEQ ID NO: 289 - skipped

SEQ ID NO: 290 - protein, 10 aa, synthetic construct
QQSYSTPPIT

SEQ ID NO: 291 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcaacacctatgctatcacctgggtgcgacaggcccctggacaagggcttgaatggatggggggaatcatccctatctctggcatagcagagtacgcacagaagttccagggcagagtcacgatcaccacggatgactcctcgaccacagcctacatggaactgaacagtctgagatctgaggacacggccgtgtattactgtgcgagctggaactacgcactctactacttctacggtatggacgtctggggccgagggaccacggtcaccgtctcctca

SEQ ID NO: 292 - protein, 122 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 293 - protein, 8 aa, synthetic construct
GGTFNTYA

SEQ ID NO: 294 - protein, 8 aa, synthetic construct
IIPISGIA

SEQ ID NO: 295 - protein, 15 aa, synthetic construct
ASWNYALYYFYGMDV

SEQ ID NO: 296 - DNA, 324 bp, synthetic construct; identical to SEQ ID NO: 236
  source 1..324: mol_type = other DNA

SEQ ID NO: 297 - protein, 108 aa, synthetic construct; identical to SEQ ID NO: 237

SEQ ID NO: 298 - protein, 7 aa, synthetic construct
QSVSSSY

SEQ ID NO: 299 - skipped

SEQ ID NO: 300 - protein, 9 aa, synthetic construct
QQYGSSPWT

SEQ ID NO: 301 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgtctattacatgaactggatccgccaggctccagggaagggcctggagtgggtttcatacattagtagtagtggtagtaccatatactacgcagactctgtgaagggccgattcaccatctccagggacaacgccaagaactcactgtatctccaaatgaacagtctgagagccgaggacacggccgtatattactgtgggagagaagggtatagtgggacttattcttattacggtatggacgtctggggccaagggaccacggtcaccgtctcctca

SEQ ID NO: 302 - protein, 123 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 303 - protein, 8 aa, synthetic construct
GFTFSVYY

SEQ ID NO: 304 - protein, 8 aa, synthetic construct
ISSSGSTI

SEQ ID NO: 305 - protein, 16 aa, synthetic construct
GREGYSGTYSYYGMDV

SEQ ID NO: 306 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcaacagagtggaggctgaggatgttggggtttattactgcatgcaagctctacaaactccgtacacttttggccaggggaccaagctggagatcaaa

SEQ ID NO: 307 - protein, 112 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIK

SEQ ID NO: 308 - protein, 11 aa, synthetic construct
QSLLHSNGYNY

SEQ ID NO: 309 - skipped

SEQ ID NO: 310 - protein, 9 aa, synthetic construct
MQALQTPYT

SEQ ID NO: 311 - DNA, 366 bp, synthetic construct
  source 1..366: mol_type = other DNA
gaagtgcagctggtggagtctgggggagggttggtacagcctggcaggtccctgagactctcctgtgcagtctctggattcacctttgatgattatgccatgcactgggtccggcaagctccagggaagggcctggagtgggtctcaggtattagttggaatagtggtaccagaggatatgcggactctgtgaagggccgattcaccatctccagagacaacgccaagaactccctgtatctgcaaatgaacagtctgagaggtgaggacacggccttgtattactgtgtaaaagatattacgatatcccccaactactacggtatggacgtctggggccaagggaccacggtcaccgtctcctca

SEQ ID NO: 312 - protein, 122 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 313 - protein, 8 aa, synthetic construct
GFTFDDYA

SEQ ID NO: 314 - protein, 8 aa, synthetic construct
ISWNSGTR

SEQ ID NO: 315 - protein, 15 aa, synthetic construct
VKDITISPNYYGMDV

SEQ ID NO: 316 - DNA, 321 bp, synthetic construct
  source 1..321: mol_type = other DNA
gacatccagatgacccagtctccatcctccctgtctgcatctgtaggagacagagtcaccatcacttgccgggcgagtcaggacattagccattattcagcctggtatcagcagaaaccagggaaacttcctaacctcctgatctatgctgcatccactttgcaatcaggggtcccatctcggttcagtggcagtggatctgggacagatttctctctcaccaccagcagcctgcagcctgaagatgttgcaacttattactgtcaaaagtataacagtgtccctctcactttcggcggagggaccaaggtggagatcaaa

SEQ ID NO: 317 - protein, 107 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIK

SEQ ID NO: 318 - protein, 6 aa, synthetic construct
QDISHY

SEQ ID NO: 319 - skipped

SEQ ID NO: 320 - protein, 9 aa, synthetic construct
QKYNSVPLT

SEQ ID NO: 321 - protein, 13 aa, synthetic construct
AHIVMVDAYKPTK

SEQ ID NO: 322 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 323 - protein, 10 aa, synthetic construct
ATHIKFSKRD

SEQ ID NO: 324 - protein, 14 aa, synthetic construct
VPTIVMVDAYKRYK

SEQ ID NO: 325 - protein, 16 aa, synthetic construct
TDKDMTITFTNKKDAE

SEQ ID NO: 326 - protein, 10 aa, synthetic construct
EQKLISEEDL

SEQ ID NO: 327 - protein, 8 aa, synthetic construct
IGTRYLTR

SEQ ID NO: 328 - protein, 4 aa, synthetic construct
GLSG

SEQ ID NO: 329 - protein, 6 aa, synthetic construct
GLSGSG

SEQ ID NO: 330 - protein, 8 aa, synthetic construct
GLSGLSGS

SEQ ID NO: 331 - protein, 10 aa, synthetic construct
GLSGLSGLSG

SEQ ID NO: 332 - protein, 10 aa, synthetic construct
GLSGGSGLSG

SEQ ID NO: 333 - protein, 243 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 334 - protein, 245 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 335 - protein, 246 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 336 - protein, 243 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 337 - protein, 238 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 338 - protein, 245 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 339 - protein, 240 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 340 - protein, 250 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 341 - protein, 245 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 342 - protein, 247 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 343 - protein, 241 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 344 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 345 - protein, 248 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 346 - protein, 242 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 347 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 348 - protein, 251 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 349 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 350 - protein, 242 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 351 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 352 - protein, 243 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 353 - protein, 243 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 354 - protein, 250 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 355 - protein, 244 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 356 - protein, 239 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 357 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 358 - protein, 249 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 359 - protein, 238 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 360 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 361 - protein, 242 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 362 - protein, 242 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 363 - protein, 245 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 364 - protein, 244 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 365 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTTVTVSS

SEQ ID NO: 366 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTTVTVSS

SEQ ID NO: 367 - protein, 122 aa, synthetic construct
EVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTMVTVSS

SEQ ID NO: 368 - protein, 107 aa, synthetic construct
DIVMTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 369 - protein, 107 aa, synthetic construct
DIQMTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 370 - protein, 120 aa, synthetic construct
QVQLVESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 371 - protein, 107 aa, synthetic construct
DILMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVEIK

SEQ ID NO: 372 - protein, 116 aa, synthetic construct
QVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 373 - protein, 107 aa, synthetic construct
DIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 374 - protein, 125 aa, synthetic construct
QVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 375 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKVEIK

SEQ ID NO: 376 - protein, 117 aa, synthetic construct
EVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 377 - protein, 121 aa, synthetic construct
EVQLVQSGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTMVTVSS

SEQ ID NO: 378 - protein, 107 aa, synthetic construct
DIQLTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 379 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTTVTVSS

SEQ ID NO: 380 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVDIK

SEQ ID NO: 381 - protein, 123 aa, synthetic construct
QVQLVESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 382 - protein, 123 aa, synthetic construct
EVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 383 - protein, 112 aa, synthetic construct
DIQLTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKVEIK

SEQ ID NO: 384 - protein, 122 aa, synthetic construct
EVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 385 - protein, 124 aa, synthetic construct
EVQLVESGAEVKKPGASVKVSCKASGYTFTSYGISWVRQAPGQGLEWMGWISVYHGNTNYAQKFZGRVTMTTDTSTSTAYMELRSLRSDDTAVYYCAREGYYDFWSGYYPFDYWGQGTTVTVSS

SEQ ID NO: 386 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTMVTVSS

SEQ ID NO: 387 - protein, 123 aa, synthetic construct
EVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 388 - protein, 107 aa, synthetic construct
DIQLTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGRTKVEIK

SEQ ID NO: 389 - protein, 108 aa, synthetic construct
MSYYHHHHHHDYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 390 - protein, 12 aa, synthetic construct
KLGDIEFIKVNK

SEQ ID NO: 391 - protein, 133 aa, synthetic construct
MGSSHHHHHHSSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 392 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 393 - protein, 735 aa, synthetic construct [sequence omitted]

SEQ ID NO: 394 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcgcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 395 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 396 - DNA, 2244 bp, synthetic construct
  source 1..2244: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcccacatcgtgatggtggacgcctacaagccgacgaagcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 397 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAHIVMVDAYKPTKQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 398 - DNA, 2253 bp, synthetic construct
  source 1..2253: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcgagcgcccacatcgtgatggtggacgcctacaagccgacgaaggcgcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 399 - protein, 750 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNASAHIVMVDAYKPTKAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 400 - DNA, 2256 bp, synthetic construct
  source 1..2256: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcagcgcccacatcgtgatggtggacgcctacaagccgacgaagggcagccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 401 - protein, 751 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSAHIVMVDAYKPTKGSQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 402 - DNA, 2268 bp, synthetic construct
  source 1..2268: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcgcccacatcgtgatggtggacgcctacaagccgacgaagggcctgagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 403 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGAHIVMVDAYKPTKGLSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 404 - DNA, 2280 bp, synthetic construct
  source 1..2280: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcagcggcgcccacatcgtgatggtggacgcctacaagccgacgaagggcctgagcggcagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 405 - protein, 759 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGSGAHIVMVDAYKPTKGLSGSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 406 - DNA, 2292 bp, synthetic construct
  source 1..2292: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcctgagcggcagcgcccacatcgtgatggtggacgcctacaagccgacgaagggcctgagcggcctgagcggcagccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 407 - protein, 763 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGLSGSAHIVMVDAYKPTKGLSGLSGSQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 408 - DNA, 2304 bp, synthetic construct
  source 1..2304: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcctgagcggcctgagcggcgcccacatcgtgatggtggacgcctacaagccgacgaagggcctgagcggcctgagcggcctgagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 409 - protein, 767 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGLSGLSGAHIVMVDAYKPTKGLSGLSGLSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 410 - DNA, 2247 bp, synthetic construct
  source 1..2247: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggagcccacatcgtgatggtggacgcctacaagccgacgaagaccaccacgcagtcaaggcttcagttttctcaggccggagcgagtgacattcgggaccagtctaggaactggcttcctggaccctgttacgcccagcaggccgtatcaaagacatctgcggataacaacaacagtgaatactcgtggactggagctaccaagtaccacctcaatggcagagactctctggtgaatccgggcccggccatggcaagccacaaggacgatgaagaagccttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 411 - protein, 748 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGAHIVMVDAYKPTKTTTQSRLQFSQAGASDIRDQSRNWLPGPCYAQQAVSKTSADNNNSEYSWTGATKYHLNGRDSLVNPGPAMASHKDDEEAFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 412 - DNA, 2307 bp, synthetic construct
  source 1..2307: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggcctgagcggcctgagcggcctgagcggcgcccacatcgtgatggtggacgcctacaagccgacgaagggcctgagcggcctgagcggcctgagcggcaccaccacgcagtcaaggcttcagttttctcaggccggagcgagtgacattcgggaccagtctaggaactggcttcctggaccctgttacgcccagcaggccgtatcaaagacatctgcggataacaacaacagtgaatactcgtggactggagctaccaagtaccacctcaatggcagagactctctggtgaatccgggcccggccatggcaagccacaaggacgatgaagaagccttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 413 - protein, 768 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGLSGLSGLSGAHIVMVDAYKPTKGLSGLSGLSGTTTQSRLQFSQAGASDIRDQSRNWLPGPCYAQQAVSKTSADNNNSEYSWTGATKYHLNGRDSLVNPGPAMASHKDDEEAFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 414 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 415 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 416 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 417 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 418 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 419 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 420 - DNA, 2250 bp, synthetic construct
  source 1..2250: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcagcccacatcgtgatggtggacgcctacaagccgacgaagcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 421 - protein, 749 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAAHIVMVDAYKPTKQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 422 - DNA, 2310 bp, synthetic construct
  source 1..2310: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcaggcctgagcagcggcagcggcctgagcggcgcccacatcgtgatggtggacgcctacaagccgacgaagggcctgagcggcggcagcggcctgagcggccaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 423 - protein, 769 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAGLSSGSGLSGAHIVMVDAYKPTKGLSGGSGLSGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 424 - DNA, 2310 bp, synthetic construct
  source 1..2310: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggtggcctgagcagcggcagcggcctgagcggcgcccacatcgtgatggtggacgcctacaagccgacgaagggcctgagcggcggcagcggcctgagcggctctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 425 - protein, 769 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGGLSSGSGLSGAHIVMVDAYKPTKGLSGGSGLSGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 426 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 427 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 428 - DNA, 2211 bp, synthetic construct
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 429 - protein, 735 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 430 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 431 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacgaacaaaaactcatctcagaagaggatctgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 432 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEQKLISEEDLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 433 - protein, 6 aa, synthetic construct
GSGESG

SEQ ID NO: 434 - protein, 760 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 435 - protein, 763 aa, synthetic construct
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 436 - protein, 672 aa, synthetic construct
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 437 - DNA, 1341 bp, synthetic construct
  source 1..1341: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaagcggcgaaagcggcgtggataccctgtccggactgagcagtgagcaaggccagtccggagatatgacaattgaagaagatagcgccacccatattaaattctccaaaagagatgaggacggcaaagagctggctggagcaacaatggagctgagagattcctctggaaagactattagtacatggatctctgatggccaagtgaaagatttctatctgtatccaggaaagtacacatttgtcgaaaccgctgcaccagacggatatgaggtggctacagctattacctttacagtgaatgagcaaggacaggtgactgttaatggcaaagctactaaaggagacgctcatatttaa

SEQ ID NO: 438 - protein, 446 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGESGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 439 - protein, 224 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGSGESGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 440 - DNA, 1020 bp, synthetic construct
  source 1..1020: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaagcggcgaaagcggcgtggataccctgtccggactg

SEQ ID NO: 441 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 442 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 443 - DNA, 22 bp, synthetic construct
  source 1..22: mol_type = other DNA
atcctcccccttgctgtcctgc

SEQ ID NO: 444 - DNA, 57 bp, synthetic construct
  source 1..57: mol_type = other DNA
tcgtcggcagcgtcagatgtgtataagagacaggcactgacaattccgtggtctagg

SEQ ID NO: 445 - DNA, 59 bp, synthetic construct
  source 1..59: mol_type = other DNA
gtctcgtgggctcggagatgtgtataagagacagcaaacaacagatggctggcaactag

SEQ ID NO: 446 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0242422 A1
(43) Pub. Date: Aug. 20, 2026

(54) VIRAL PARTICLES RETARGETED TO TRANSFERRIN RECEPTOR 1

(71) Applicant: Regeneron Pharmaceuticals, Inc., Tarrytown, NY (US)

(72) Inventors: Leah Sabin, Goldens Bridge, NY (US); Christos Kyratsous, Irvington, NY (US); Kyle Plasterer, White Plains, NY (US); John Dugan, Hoboken, NJ (US); Min Gao, Woodcliff Lake, NJ (US); Pascaline Aime-Wilson, New York, NY (US); Nicole Keating, Yorktown Heights, NY (US); Robert Babb, River Edge, NJ (US); Maria Praggastis, Cortlandt Manor, NY (US); Katherine Cygnar, New York, NY (US)

(21) Appl. No.: 19/099,297

(22) PCT Filed: Jul. 28, 2023
(86) PCT No.: PCT/US2023/071268

§ 371 Date: Jan. 28, 2025

Related U.S. Application Data
(60) Provisional application No. 63/393,741, filed on Jul. 29, 2022.
(60) Provisional application No. 63/502,562, filed on May 16, 2023.

(51) Int. Cl. C07K 14/005; C07K 16/28; C12N 15/86

(52) U.S. Cl. CPC C07K 14/005; C07K 16/2881; C12N 15/86; C07K 2317/35; C07K 2317/55; C07K 2317/622; C07K 2319/00; C12N 2750/14122; C12N 2750/14143; C12N 2750/14145

(57) ABSTRACT

Provided herein are compositions and methods for retargeting viral particles, e.g., adeno-associated virus (AAV) particles, to TfR1 expressing cells, including blood brain barrier (BBB) endothelial cells. AAV adapted accordingly may be a viable gene therapy platform for gene therapy of target cells that express TfR1, e.g., for gene therapy across the blood-brain barrier in a patient in need thereof.

### TECHNICAL FIELDS

The disclosure herein relates to methods of making and using recombinant viral particles, e.g., recombinant AAV particles, comprising capsid proteins retargeted to a cell surface protein that allows the viral particles to bind transferrin receptor 1 (also referred to in this application as TfR or CD71; encoded by TFRC), which may be (a) useful for infection (e.g., gene modification) of cells that express TfR and/or (b) transcytosis of the AAV particle across cells that express TfR, such as blood brain barrier endothelial cells, in vitro or in vivo.

### SEQUENCE LISTING

A Sequence Listing in xml format entitled “11081WO01_xml.xml,” which was created Jul. 28, 2023, and is 461 Kb, is incorporated herein by reference in its entirety.

### BACKGROUND OF THE INVENTION

The delivery of genes into particular target cells has become one of the most important technologies in modern medicine for the potential treatment of a variety of chronic and genetic diseases. Ideally, a gene delivery vehicle is able to stably introduce genetic material into desired cells and avoid introducing genetic material into non-target cells.

Viral particles, particularly those based on adeno-associated virus (AAV), as gene delivery vehicles have been the focus of much research since AAVs are capable of transducing a wide range of primate species and tissues in vivo with no evidence of pathogenicity. (Muzyczka, et al. (1992) Current Topics in Microbiology and Immunology, 158:97-129). Moreover, AAV safely transduces postmitotic tissues. Although the virus can occasionally integrate into host chromosomes, it does so very infrequently into a safe-harbor locus in human chromosome 19, and only when the replication (Rep) proteins are supplied in trans. AAV genomes rapidly circularize and concatemerize in infected cells, and exist in a stable, episomal state in infected cells to provide long-term stable expression of their payloads.

Additionally, manipulating and redirecting AAV infection to specific cells has been achieved in recent years. Many of the advances in targeted gene therapy using viral particles may be summarized as non-recombinatorial (non-genetic) or recombinatorial (genetic) modification of the viral particle, which result in the pseudotyping, expanding, and/or retargeting of the natural tropism of the viral particle. (Reviewed in Nicklin and Baker (2002) Curr. Gene Ther. 2:273-93; Verheiji and Rottier (2012) Advances Virol 2012:1-15).

In a direct recombinatorial targeting approach, a targeting ligand is directly inserted into, or coupled to, a viral capsid, i.e., protein viral capsid genes are modified to express capsid proteins comprising a heterologous targeting ligand. The targeting ligand than redirects, e.g., binds, a receptor or marker preferentially or exclusively expressed on a target cell. (Stachler et al. (2006) Gene Ther. 13:926-931; White et al. (2004) Circulation 109:513-519; see also Park et al., (2007) Frontiers in Bioscience 13:2653-59; Girod et al. (1999) Nature Medicine 5:1052-56; Grifman et al. (2001) Molecular Therapy 3:964-75; Shi et al. (2001) Human Gene Therapy 12:1697-1711; Shi and Bartlett (2003) Molecular Therapy 7:515-525).

In indirect recombinatorial approaches, a viral capsid is modified with a heterologous “scaffold”, which then links to an adaptor that includes a targeting ligand. The adaptor binds to the scaffold and the target cell. (Arnold et al. (2006)Mol. Ther. 5:125-132; Ponnazhagen et al. (2002) J. Virol. 76:12900-907; see also WO 97/05266) Scaffolds such as (1) Fc binding molecules (e.g., Fc receptors, Protein A, etc.), which bind to the Fc of antibody adaptors, (2) (strept)avidin, which binds to biotinylated adaptors, (3) biotin, which binds to adaptors fused with (strept)avidin, (4) a detectable label, which is useful for detection and/or isolation of viral particles, bound by a bispecific adaptor able to non-covalently bind the detectable label and target molecule, and recently (5) protein:protein binding pairs that form isopeptide bonds have been described for a variety of viral particles. (See, e.g., Gigout et al. (2005) Molecular Therapy 11:856-865; Stachler et al. (2008) Molecular Therapy 16:1467-1473; Quetglas et al. (2010) Virus Research 153:179-196; Ohno et al. (1997) Nature Biotechnology 15:763-767; Klimstra et al. (2005) Virology 338:9-21).

With the advances providing the ability to direct AAV infection, there remains a need for viral systems that are adaptable for the targeted transfer of nucleic acids of interest to a target cell or receptor.

### SUMMARY OF THE INVENTION

It is shown herein that an AAV capsid protein may be modified to allow for the targeted introduction of a nucleotide of interest into mammalian cells that express transferrin receptor 1 (TfR1; CD71) and/or for the crossing of the modified AAV capsid across the blood brain barrier (BBB) via BBB endothelial cells that express TfR1.

Viral particles as described herein are particularly suited for the targeted introduction of a nucleotide of interest specifically to a cell expressing transferrin receptor 1 (TfR1; CD71) or across the blood brain barrier since the viral capsid or viral capsid protein(s) described herein comprise a first member of a protein:protein binding pair, associated with its cognate second member of the protein:protein binding pair, wherein the second member is linked (e.g., fused to) a targeting ligand that binds a transferrin receptor 1 (TfR1) that is expressed on a cell surface.

Described herein are recombinant viral capsid proteins, and viral particles (e.g., wherein the viral capsid proteins encapsidates a nucleic acid of interest) and compositions comprising the viral capsid proteins and/or viral particles, e.g., pharmaceutical compositions comprising the viral capsid proteins and/or viral particles, wherein a recombinant viral capsid protein as described herein comprises: (i) a first member of a protein:protein binding pair inserted and/or displayed by the viral capsid, (ii) a second member of the protein:protein binding pair, wherein the first member of the protein:protein binding pair and the second member of the protein:protein binding pair are associated, and (iii) an antibody or binding portion thereof that binds an extracellular domain of a transferrin receptor protein 1 (abbreviated TfR1, TfR or CD71), wherein the antibody or binding portion thereof is fused to the second member of the protein:protein binding pair. In some embodiments, (i) the first member of the protein:protein binding pair, (ii) the second member of the protein:protein binding pair, and (iii) the antibody or binding portion thereof together direct the tropism of the viral capsid to a cell that expresses the TfR1, or an extracellular portion thereof, e.g., an amino acid sequence set forth in SEQ ID NO:436. In some embodiments, the extracellular domain of TfR1 is an extracellular domain of human (h) TfR1.

In some embodiments, the viral capsid protein/viral particle and/or composition comprising the same further comprises a cell that expresses TfR1 on its surface, e.g., wherein the viral capsid is bound to an extracellular domain of the TfR1 expressed on the surface of the cell. In some embodiments, the cell is a cell selected from the group of cells listed in Table 2, optionally wherein the cell is in vivo, ex vivo, or in vitro. In some embodiments, an AAV viral capsid protein displaying a TfR1 targeting ligand as disclosed herein (including a viral particle and/or composition comprising the same) is bound to a cell that expresses TfR1 on its surface, wherein the cell is a central nervous system cell, such as a cortical neuron, purkinje cell, a glial cell (e.g., an astrocyte, oligodendrocyte, etc.), and the like. In some embodiments, the cell is a blood brain barrier endothelial cell and/or a brain microvascular endothelial that expresses TfR1 on its surface, wherein the viral capsid is bound to an extracellular domain of the TfR1 expressed on the surface of the blood brain barrier endothelial cell and/or a brain microvascular endothelial, optionally wherein the blood brain barrier endothelial cell and/or a brain microvascular endothelial is in vivo, ex vivo, or in vitro.

Protein:protein binding pairs described herein are those protein:protein binding pairs that spontaneously form an isopeptide bond upon contact. In some embodiments:
(a) the first member of the protein:protein binding pair comprises SpyTag, Isopeptag, SnoopTag, SpyTag002, SpyTag003, or variants thereof,
(b) the second member of the protein:protein binding pair comprises, fused to the targeting ligand, a SpyCatcher, KTag, pilin-C, SnoopCatcher, SpyCatcher002, SpyTag003, or variants thereof, and
(c) the first member of the protein:protein binding pair and the second member of the protein:protein binding pair are associated by an isopeptide bond. In some embodiments, (a) the first member of the protein:protein binding pair comprises SpyTag, or a variant thereof, and (b) the second member of the protein:protein binding pair comprises SpyCatcher, or a variant thereof, fused to the targeting ligand. In some embodiments, (a) the first member of the protein:protein binding pair comprises the c-myc amino acid sequence set forth as SEQ ID NO:326, and (b) the second member of the protein:protein binding pair comprises a bispecific binding protein comprising an anti-c-myc antibody and the targeting ligand.

In some further embodiments, the viral capsid protein further comprises a linker that flanks one or both sides of the first member of the protein:protein binding pair. In some embodiments, a first and/or second linker operably linking the first member of the protein:protein binding pair to a capsid protein of the viral capsid are each independently at least one amino acid in length (e.g., 10 amino acids in length), and are not identical or identical. In some embodiments, the first linker is 10 amino acids in length and/or the second linker is 10 amino acids in length, optionally wherein the amino acid sequence of the first linker and/or the amino acid sequence of the second linker comprises the amino acid sequence set forth as SEQ ID NO:331 or SEQ ID NO:332.

In some recombinant viral capsid protein embodiments, the viral capsid protein comprises an amino acid sequence of a modified VP1 capsid protein, a modified VP2 capsid protein, and/or modified VP3 capsid protein encoded by a mutated cap gene, and the mutated cap gene or a portion thereof (e.g., about 15 nucleotides) comprises a nucleotide sequence at least 90% identical to a cap gene of an AAV or portion thereof, wherein the mutated cap gene or portion thereof is genetically modified to comprise an insertion of a nucleotide sequence encoding the first member of the protein:protein binding pair such that the modified VP1 capsid protein, the modified VP2 capsid protein and/or the modified VP3 capsid protein comprises the first member of the protein:protein binding pair. In some embodiments, the mutated cap gene or portion thereof is genetically modified to comprise one or more additional mutations such that the modified VP1 capsid protein, the modified VP2 capsid protein, and/or the modified VP3 capsid protein comprises, in addition to the first member of the protein:protein binding pair:
(i) a point mutation, e.g., a substitution, insertion, or deletion of an amino acid,
(ii) a chimeric amino acid sequence, or
(iii) both a point mutation and a chimeric amino acid sequence, optionally wherein the substitution, insertion, or deletion of an amino acid reduces the natural tropism of the viral particle and/or creates a detectable label. In some recombinant viral capsid protein embodiments, (a) the viral capsid protein comprises an amino acid sequence of a modified VP1 capsid protein, a modified VP2 capsid protein, and/or modified VP3 capsid protein encoded by a mutated cap gene, (b) the mutated cap gene or a portion thereof (e.g., at least 5 nucleotides, at least 15 nucleotides, at least 30 nucleotides, etc.) comprises a nucleotide sequence at least 90% identical to a cap gene of an AAV or portion thereof, wherein the mutated cap gene or portion thereof is genetically modified to comprise an insertion of a nucleotide sequence encoding the first member of the protein:protein binding pair such that the modified VP1 capsid protein, the modified VP2 capsid protein and/or the modified VP3 capsid protein comprises the first member of the protein:protein binding pair, and/or (c) the AAV is selected from the group consisting of AAV1, AAV2, AAV3B, AAV4, AAV5, AAV6, AAV7, AAV8, AAV9, AAV rh10, AAV rh32.33, a non-primate animal AAV listed in Table 5, a combination thereof, and any variant or hybrid thereof.

In some embodiments, the AAV is AAV2, and optionally, the viral capsid comprises modified AAV2 VP1 capsid proteins that comprise the first member of the protein:protein binding pair linked, optionally via a linker, to an amino acid at position 1453 and/or 1587. In some embodiments, the viral capsid comprises modified AAV2 VP1 capsid proteins that comprise the first member of the protein:protein binding pair displayed, via a linker, at position G453, optionally wherein the modified AAV2 VP1 capsid proteins further comprise an R585A modification, an R588A modification, or both the R585A modification and the R588A modification, and optionally wherein the modified AAV2 VP1 capsid proteins further comprise an R484A modification, an R487A modification, an R585A modification, an R588A modification, and an K532A modification, or any combination of an R484A modification, an R487A modification, an R585A modification, an R588A modification, and an K532A modification.

In some embodiments, the AAV is AAV9, and optionally, the viral capsid comprises modified AAV9 VP1 capsid proteins that comprise the first member of the protein:protein binding pair linked, optionally via a linker, to an amino acid at position 1453 or 1589. In some embodiments, the viral capsid comprises modified AAV9 VP1 capsid proteins that comprise the first member of the protein:protein binding pair displayed, via a linker, at G453, optionally wherein the modified AAV9 VP1 capsid proteins further comprise an N272A modification, a W503A modification, or both the N272A modification and the W503A modification. In some embodiments, the recombinant viral capsid the viral capsid is a mosaic viral capsid comprising a second set of AAV9 VP1 capsid proteins lacking the first member of the protein:protein binding pair, optionally wherein the second set of AAV2 VP1 capsid proteins comprise an N272A modification, a W503A modification, or both the N272A modification and the W503A modification.

In some embodiments, the AAV is AAV1, and optionally, the viral capsid comprises modified AAV1 VP1 capsid proteins that comprise the first member of the protein:protein binding pair linked, optionally via a linker. In some embodiments, the recombinant viral capsid the viral capsid is a mosaic viral capsid comprising a second set of AAV1 VP1 capsid proteins lacking the first member of the protein:protein binding pair.

In some embodiments, the AAV is AAV8, and optionally, the viral capsid comprises modified AAV8 VP1 capsid proteins that comprise the first member of the protein:protein binding pair linked, optionally via a linker. In some embodiments, the recombinant viral capsid the viral capsid is a mosaic viral capsid comprising a second set of AAV8 VP1 capsid proteins lacking the first member of the protein:protein binding pair.

In some embodiments, the AAV is AAVrh32.33, and optionally, the viral capsid comprises modified AAVrh32.33 VP1 capsid proteins that comprise the first member of the protein:protein binding pair linked, optionally via a linker. In some embodiments, the recombinant viral capsid the viral capsid is a mosaic viral capsid comprising a second set of AAV rh32.33 VP1 capsid proteins lacking the first member of the protein:protein binding pair.

In some embodiments, the non-primate animal AAV is an avian AAV (AAAV), a non-human mammalian AAV or a squamate AAV.

In some embodiments, the non-primate animal AAV is an AAAV, and optionally, the viral capsid comprises modified AAAV VP1 capsid proteins that comprise the first member of the protein:protein binding pair linked, optionally via a linker, to an amino acid at position 1444 or 1580. In some embodiments, the viral capsid comprises modified AAAV VP1 capsid proteins that comprise the first member of the protein:protein binding pair linked, optionally via a linker, to an amino acid at a position selected from the group consisting of I429, I430, I431, I432, I433, I434, I436, I437, and I565.

In some embodiments, the AAV is a squamate AAV, e.g., bearded dragon AAV. In some embodiments, the viral capsid comprises modified bearded dragon VP1 capsid proteins that comprise the first member of the protein:protein binding pair linked, optionally via a linker, to an amino acid at position I573 or I436.

In some embodiments, the AAV is a sea lion AAV.

An anti-TfR binding protein, e.g., anti-TfR antibody or binding portion thereof, may be used to retarget a recombinant AAV viral capsid protein/AAV viral particle to a cell that expresses a TfR (e.g., a human TfR). In some embodiments, the antibody or binding portion thereof that binds an extracellular domain of TfR1 binds the same epitope on the extracellular domain of TfR1 as a reference antibody comprising an HCVR/LCVR amino acid sequence pair as set forth in Table 1; comprises heavy chain complementarity determining regions (HCDR1, HCDR2 and HCDR3) from a heavy chain variable region (HCVR) comprising an amino acid sequence set forth in SEQ ID NO: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302 or 312 (or a variant thereof); and/or comprises light chain complementarity determining regions (LCDR1, LCDR2 and LCDR3) from a light chain variable region (LCVR) comprising an amino acid sequence set forth in SEQ ID NO: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307 or 317 (or a variant thereof). In some embodiments, the antibody or binding portion thereof that binds TfR1, e.g., human TfR1, and which may be used to retarget an AAV viral particle as described herein comprises:
(i) a HCVR comprising the HCDR1, HCDR2 and HCDR3 of a HCRV that comprises the amino acid sequence set forth in SEQ ID NO: 2 (or a variant thereof); and a LCVR comprising the LCDR1, LCDR2 and LCDR3 of a LCRV that comprises the amino acid sequence set forth in SEQ ID NO: 7 (or a variant thereof);
(ii) a HCVR comprising the HCDR1, HCDR2 and HCDR3 of a HCRV that comprises the amino acid sequence set forth in SEQ ID NO: 12 (or a variant thereof); and a LCVR comprising the LCDR1, LCDR2 and LCDR3 of a LCRV that comprises the amino acid sequence set forth in SEQ ID NO: 17 (or a variant thereof);
(iii) a HCVR comprising the HCDR1, HCDR2 and HCDR3 of a HCRV that comprises the amino acid sequence set forth in SEQ ID NO: 22 (or a variant thereof); and a LCVR comprising the LCDR1, LCDR2 and LCDR3 of a LCRV that comprises the amino acid sequence set forth in SEQ ID NO: 27 (or a variant thereof);
(iv) a HCVR comprising the HCDR1, HCDR2 and HCDR3 of a HCRV that comprises the amino acid sequence set forth in SEQ ID NO: 32 (or a variant thereof); and a LCVR comprising the LCDR1, LCDR2 and LCDR3 of a LCRV that comprises the amino acid sequence set forth in SEQ ID NO: 37 (or a variant thereof);
(v) a HCVR comprising the HCDR1, HCDR2 and HCDR3 of a HCRV that comprises the amino acid sequence set forth in SEQ ID NO: 42 (or a variant thereof); and a LCVR comprising the LCDR1, LCDR2 and LCDR3 of a LCRV that comprises the amino acid sequence set forth in SEQ ID NO: 47 (or a variant thereof);
(vi) a HCVR comprising the HCDR1, HCDR2 and HCDR3 of a HCRV that comprises the amino acid sequence set forth in SEQ ID NO: 52 (or a variant thereof); and a LCVR comprising the LCDR1, LCDR2 and LCDR3 of a LCRV that comprises the amino acid sequence set forth in SEQ ID NO: 57 (or a variant thereof);
(vii) a HCVR comprising the HCDR1, HCDR2 and HCDR3 of a HCRV that comprises the amino acid sequence set forth in SEQ ID NO: 62 (or a variant thereof); and a LCVR comprising the LCDR1, LCDR2 and LCDR3 of a LCRV that comprises the amino acid sequence set forth in SEQ ID NO: 67 (or a variant thereof);
(viii) a HCVR comprising the HCDR1, HCDR2 and HCDR3 of a HCRV that comprises the amino acid sequence set forth in SEQ ID NO: 72 (or a variant thereof); and a LCVR comprising the LCDR1, LCDR2 and LCDR3 of a LCRV that comprises the amino acid sequence set forth in SEQ ID NO: 77 (or a variant thereof);
(ix) a HCVR comprising the HCDR1, HCDR2 and HCDR3 of a HCRV that comprises the amino acid sequence set forth in SEQ ID NO: 82 (or a variant thereof); and a LCVR comprising the LCDR1, LCDR2 and LCDR3 of a LCRV that comprises the amino acid sequence set forth in SEQ ID NO: 87 (or a variant thereof);
(x) a HCVR comprising the HCDR1, HCDR2 and HCDR3 of a HCRV that comprises the amino acid sequence set forth in SEQ ID NO: 92 (or a variant thereof); and a LCVR comprising the LCDR1, LCDR2 and LCDR3 of a LCRV that comprises the amino acid sequence set forth in SEQ ID NO: 97 (or a variant thereof);
(xi) a HCVR comprising the HCDR1, HCDR2 and HCDR3 of a HCRV that comprises the amino acid sequence set forth in SEQ ID NO: 102 (or a variant thereof); and a LCVR comprising the LCDR1, LCDR2 and LCDR3 of a LCRV that comprises the amino acid sequence set forth in SEQ ID NO: 107 (or a variant thereof);
(xii) a HCVR comprising the HCDR1, HCDR2 and HCDR3 of a HCRV that comprises the amino acid sequence set forth in SEQ ID NO: 112 (or a variant thereof); and a LCVR comprising the LCDR1, LCDR2 and LCDR3 of a LCRV that comprises the amino acid sequence set forth in SEQ ID NO: 117 (or a variant thereof);
(xiii) a HCVR comprising the HCDR1, HCDR2 and HCDR3 of a HCRV that comprises the amino acid sequence set forth in SEQ ID NO: 122 (or a variant thereof); and a LCVR comprising the LCDR1, LCDR2 and LCDR3 of a LCRV that comprises the amino acid sequence set forth in SEQ ID NO: 127 (or a variant thereof);
(xiv) a HCVR comprising the HCDR1, HCDR2 and HCDR3 of a HCRV that comprises the amino acid sequence set forth in SEQ ID NO: 132 (or a variant thereof); and a LCVR comprising the LCDR1, LCDR2 and LCDR3 of a LCRV that comprises the amino acid sequence set forth in SEQ ID NO: 137 (or a variant thereof);
(xv) a HCVR comprising the HCDR1, HCDR2 and HCDR3 of a HCRV that comprises the amino acid sequence set forth in SEQ ID NO: 142 (or a variant thereof); and a LCVR comprising the LCDR1, LCDR2 and LCDR3 of a LCRV that comprises the amino acid sequence set forth in SEQ ID NO: 147 (or a variant thereof);
(xvi) a HCVR comprising the HCDR1, HCDR2 and HCDR3 of a HCRV that comprises the amino acid sequence set forth in SEQ ID NO: 152 (or a variant thereof); and a LCVR comprising the LCDR1, LCDR2 and LCDR3 of a LCRV that comprises the amino acid sequence set forth in SEQ ID NO: 157 (or a variant thereof);
(xvii) a HCVR comprising the HCDR1, HCDR2 and HCDR3 of a HCRV that comprises the amino acid sequence set forth in SEQ ID NO: 162 (or a variant thereof); and a LCVR comprising the LCDR1, LCDR2 and LCDR3 of a LCRV that comprises the amino acid sequence set forth in SEQ ID NO: 167 (or a variant thereof);
(xviii) a HCVR comprising the HCDR1, HCDR2 and HCDR3 of a HCRV that comprises the amino acid sequence set forth in SEQ ID NO: 172 (or a variant thereof); and a LCVR comprising the LCDR1, LCDR2 and LCDR3 of a LCRV that comprises the amino acid sequence set forth in SEQ ID NO: 177 (or a variant thereof);
(xix) a HCVR comprising the HCDR1, HCDR2 and HCDR3 of a HCRV that comprises the amino acid sequence set forth in SEQ ID NO: 182 (or a variant thereof); and a LCVR comprising the LCDR1, LCDR2 and LCDR3 of a LCRV that comprises the amino acid sequence set forth in SEQ ID NO: 187 (or a variant thereof);
(xx) a HCVR comprising the HCDR1, HCDR2 and HCDR3 of a HCRV that comprises the amino acid sequence set forth in SEQ ID NO: 192 (or a variant thereof); and a LCVR comprising the LCDR1, LCDR2 and LCDR3 of a LCRV that comprises the amino acid sequence set forth in SEQ ID NO: 197 (or a variant thereof);
(xxi) a HCVR comprising the HCDR1, HCDR2 and HCDR3 of a HCRV that comprises the amino acid sequence set forth in SEQ ID NO: 202 (or a variant thereof); and a LCVR comprising the LCDR1, LCDR2 and LCDR3 of a LCRV that comprises the amino acid sequence set forth in SEQ ID NO: 207 (or a variant thereof);
(xxii) a HCVR comprising the HCDR1, HCDR2 and HCDR3 of a HCRV that comprises the amino acid sequence set forth in SEQ ID NO: 212 (or a variant thereof); and a LCVR comprising the LCDR1, LCDR2 and LCDR3 of a LCRV that comprises the amino acid sequence set forth in SEQ ID NO: 217 (or a variant thereof);
(xxiii) a HCVR comprising the HCDR1, HCDR2 and HCDR3 of a HCRV that comprises the amino acid sequence set forth in SEQ ID NO: 222 (or a variant thereof); and a LCVR comprising the LCDR1, LCDR2 and LCDR3 of a LCRV that comprises the amino acid sequence set forth in SEQ ID NO: 227 (or a variant thereof);
(xiv) a HCVR comprising the HCDR1, HCDR2 and HCDR3 of a HCRV that comprises the amino acid sequence set forth in SEQ ID NO: 232 (or a variant thereof); and a LCVR comprising the LCDR1, LCDR2 and LCDR3 of a LCRV that comprises the amino acid sequence set forth in SEQ ID NO: 237 (or a variant thereof);
(xv) a HCVR comprising the HCDR1, HCDR2 and HCDR3 of a HCRV that comprises the amino acid sequence set forth in SEQ ID NO: 242 (or a variant thereof); and a LCVR comprising the LCDR1, LCDR2 and LCDR3 of a LCRV that comprises the amino acid sequence set forth in SEQ ID NO: 247 (or a variant thereof);
(xvi) a HCVR comprising the HCDR1, HCDR2 and HCDR3 of a HCRV that comprises the amino acid sequence set forth in SEQ ID NO: 252 (or a variant thereof); and a LCVR comprising the LCDR1, LCDR2 and LCDR3 of a LCRV that comprises the amino acid sequence set forth in SEQ ID NO: 257 (or a variant thereof);
(xvii) a HCVR comprising the HCDR1, HCDR2 and HCDR3 of a HCRV that comprises the amino acid sequence set forth in SEQ ID NO: 262 (or a variant thereof); and a LCVR comprising the LCDR1, LCDR2 and LCDR3 of a LCRV that comprises the amino acid sequence set forth in SEQ ID NO: 267 (or a variant thereof);
(xviii) a HCVR comprising the HCDR1, HCDR2 and HCDR3 of a HCRV that comprises the amino acid sequence set forth in SEQ ID NO: 272 (or a variant thereof); and a LCVR comprising the LCDR1, LCDR2 and LCDR3 of a LCRV that comprises the amino acid sequence set forth in SEQ ID NO: 277 (or a variant thereof);
(xix) a HCVR comprising the HCDR1, HCDR2 and HCDR3 of a HCRV that comprises the amino acid sequence set forth in SEQ ID NO: 282 (or a variant thereof); and a LCVR comprising the LCDR1, LCDR2 and LCDR3 of a LCRV that comprises the amino acid sequence set forth in SEQ ID NO: 287 (or a variant thereof);
(xxx) a HCVR comprising the HCDR1, HCDR2 and HCDR3 of a HCRV that comprises the amino acid sequence set forth in SEQ ID NO: 292 (or a variant thereof); and a LCVR comprising the LCDR1, LCDR2 and LCDR3 of a LCRV that comprises the amino acid sequence set forth in SEQ ID NO: 297 (or a variant thereof);
(xxxi) a HCVR comprising the HCDR1, HCDR2 and HCDR3 of a HCRV that comprises the amino acid sequence set forth in SEQ ID NO: 302 (or a variant thereof); and a LCVR comprising the LCDR1, LCDR2 and LCDR3 of a LCRV that comprises the amino acid sequence set forth in SEQ ID NO: 307 (or a variant thereof); and/or
(xxxii) a HCVR comprising the HCDR1, HCDR2 and HCDR3 of a HCRV that comprises the amino acid sequence set forth in SEQ ID NO: 312 (or a variant thereof); and a LCVR comprising the LCDR1, LCDR2 and LCDR3 of a LCRV that comprises the amino acid sequence set forth in SEQ ID NO: 317 (or a variant thereof).

In some embodiments, the antibody or binding portion thereof that binds a TfR1 protein, e.g., a human TfR1 protein, and which may be used to retarget an AAV viral particle as described herein comprises:
(a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof);
(b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof);
(c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof);
(d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof);
(e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof);
(f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof);
(g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof);
(h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof);
(i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof);
(j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof);
(k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof);
(l) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof);
(m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof);
(n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof);
(o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof);
(p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof);
(q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof);
(r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof);
(s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof);
(t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof);
(u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof);
(v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof);
(w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof);
(x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof);
(y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof);
(z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof);
(aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof);
(ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof);
(ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof);
(ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof);
(ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or
(af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof).

In some embodiments, an antibody or binding portion thereof that binds a TfR1 protein, e.g., a human TfR1 protein, and which may be used to retarget an AAV viral particle as described herein comprises:
(i) a HCVR that comprises the amino acid sequence set forth in SEQ ID NO: 2 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 (or a variant thereof);
(vi) a HCVR that comprises the amino acid sequence set forth in SEQ ID NO: 52 (or a variant thereof); and a LCVR that comprises the amino acid sequence set forth in SEQ ID NO: 57 (or a variant thereof);
(vii) a HCVR that comprises the amino acid sequence set forth in SEQ ID NO: 62 (or a variant thereof); and a LCVR that comprises the amino acid sequence set forth in SEQ ID NO: 67 (or a variant thereof);
(viii) a HCVR that comprises the amino acid sequence set forth in SEQ ID NO: 72 (or a variant thereof); and a LCVR that comprises the amino acid sequence set forth in SEQ ID NO: 77 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 (or a variant thereof); and a LCVR that comprises the amino acid sequence set forth in SEQ ID NO: 117 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 (or a variant thereof); and a LCVR that comprises the amino acid sequence set forth in SEQ ID NO: 147 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 (or a variant thereof); and a LCVR that comprises the amino acid sequence set forth in SEQ ID NO: 187 (or a variant thereof);
(xx) a HCVR that comprises the amino acid sequence set forth in SEQ ID NO: 192 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ TD NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 (or a variant thereof); and a LCVR that comprises the amino acid sequence set forth in SEQ ID NO: 307 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

In some embodiments, the antibody or binding portion thereof that binds an extracellular domain of TfR1 is in a bivalent monoclonal antibody or mAb format. In some embodiments, the antibody or binding portion thereof that binds an extracellular domain of TfR1 is in a Fab format. In some embodiments, the antibody or binding portion thereof that binds an extracellular domain of TfR1 is in an scFv format. In some embodiments, the antibody or binding portion thereof that binds an extracellular domain of TfR1 comprises a mutated Ig CH3 domain, wherein the mutated Ig CH3 domain binds the extracellular domain of TfR1.

In some embodiments, an antibody or binding portion thereof that binds a TfR1 protein, e.g., a human TfR1 protein, and which may be used to retarget an AAV viral particle as described herein comprises (i) a HCVR that comprises the amino acid sequence set forth in SEQ ID NO: 2 (or a variant thereof); and a LCVR that comprises the amino acid sequence set forth in SEQ ID NO: 7 (or a variant thereof); (ii) a HCVR that comprises the amino acid sequence set forth in SEQ ID NO: 42 (or a variant thereof); and a LCVR that comprises the amino acid sequence set forth in SEQ ID NO: 47 (or a variant thereof); (iii) a HCVR that comprises the amino acid sequence set forth in SEQ ID NO: 122 (or a variant thereof); and a LCVR that comprises the amino acid sequence set forth in SEQ ID NO: 127 (or a variant thereof); (iv) a HCVR that comprises the amino acid sequence set forth in SEQ ID NO: 132 (or a variant thereof); and a LCVR that comprises the amino acid sequence set forth in SEQ ID NO: 137 (or a variant thereof); (v) a HCVR that comprises the amino acid sequence set forth in SEQ ID NO: 212 (or a variant thereof); and a LCVR that comprises the amino acid sequence set forth in SEQ ID NO: 217 (or a variant thereof); (vi) a HCVR that comprises the amino acid sequence set forth in SEQ ID NO: 222 (or a variant thereof); and a LCVR that comprises the amino acid sequence set forth in SEQ ID NO: 227 (or a variant thereof); (vii) a HCVR that comprises the amino acid sequence set forth in SEQ ID NO: 232 (or a variant thereof); and a LCVR that comprises the amino acid sequence set forth in SEQ ID NO: 237 (or a variant thereof); (viii) a HCVR that comprises the amino acid sequence set forth in SEQ ID NO: 242 (or a variant thereof); and a LCVR that comprises the amino acid sequence set forth in SEQ ID NO: 247 (or a variant thereof); (ix) a HCVR that comprises the amino acid sequence set forth in SEQ ID NO: 262 (or a variant thereof); and a LCVR that comprises the amino acid sequence set forth in SEQ ID NO: 267 (or a variant thereof); (x) a HCVR that comprises the amino acid sequence set forth in SEQ ID NO: 272 (or a variant thereof); and a LCVR that comprises the amino acid sequence set forth in SEQ ID NO: 277 (or a variant thereof); (xi) a HCVR that comprises the amino acid sequence set forth in SEQ ID NO: 282 (or a variant thereof); and a LCVR that comprises the amino acid sequence set forth in SEQ ID NO: 287 (or a variant thereof); and/or (xii) a HCVR that comprises the amino acid sequence set forth in SEQ ID NO: 292 (or a variant thereof); and a LCVR that comprises the amino acid sequence set forth in SEQ ID NO: 297 (or a variant thereof).

Viral particles, e.g., viral capsids, comprising a viral capsid protein as described herein are also described. In some embodiments, a viral capsid as described herein is a mosaic capsid and further comprises a reference viral capsid protein that is at least 95% identical to the recombinant viral capsid protein, and the reference viral capsid protein lacks all three of (i) the first member of the protein:protein binding pair, (ii) the second member of the protein:protein binding pair, and (iii) the antibody or binding portion thereof. In some embodiments, the mosaic viral capsid comprises the reference capsid protein and the recombinant viral capsid protein at a ratio of at least 2:1, 3:1, 4:1, 5:1, 6:1, 7:1. 8:1, 9:1, 10:1, 11:1, 12:1, 13:1, 14:1, 15:1, 16:1, 17:1, 18:1, 19:1, 20:1. In some embodiments, the viral capsid further comprises a nucleotide of interest encapsidated within the viral capsid. In some embodiments, the nucleotide of interest is a reporter gene, e.g., the nucleotide of interest encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

In some embodiments, the nucleotide of interest encodes a therapeutic moiety, e.g., a therapeutic protein, a suicide gene, an antibody or a fragment thereof, a CRISPR/Cas system or a portion(s) thereof, an antisense oligonucleotide, a ribozyme, an RNAi molecule, or a shRNA molecule. In some embodiments, the nucleotide of interest is operably linked to a promoter that is organ-specific, tissue-specific, or cell-specific. In some embodiments, the nucleotide of interest encodes a therapeutic moiety that may be secreted from a transduced cell, and that may provide a therapeutic effect in the interstitial space surrounding the transduced cell or on a neighboring cell. In some embodiments, the nucleotide of interest encodes a therapeutic moiety that may provide a therapeutic effect in an autonomous manner to the transduced cell. In some embodiments, the nucleotide of interest encodes a therapeutic moiety that may provide a therapeutic benefit in an autonomous manner to the transduced cell, within the interstitial space surrounding the transduced cell, and/or to a cell neighboring the transduced cell. In some embodiments, the therapeutic moiety is a therapeutic protein, e.g., a fusion protein, an enzyme, etc. In some embodiments, the therapeutic moiety is a secreted antibody or binding portion thereof. In some embodiments, the therapeutic moiety is an RNA molecule, e.g., an antisense RNA molecule, and RNAi, molecule, an shRNA molecule, etc.

In some embodiments, the promoter is brain-specific. In some embodiments, the promoter is neuron-specific, glial cell-specific, astrocyte-specific, oligodendrocyte-specific, microglia-specific and/or central nervous system-specific. In some embodiments, the promoter is selected from the group consisting of human glial fibrillary acidic protein (GFAP) promoter, human synapsin 1 (SYN1) promoter, human synapsin 2 (SYN2) promoter, human metallothionein 3 (MT3) promoter, and human proteolipid protein 1 (PLP1) promoter. In some embodiments, the promoter is a neuron-, astrocyte-, or oligodendrocyte-specific, or neuron-, astrocyte-, or oligodendrocyte-preferential promoter. In some embodiments, the promoter is selected from the group consisting of: an NSE promoter, a Synapsin promoter, a MeCP2 promoter, an oligodendrocyte transcription factor 1 (Olig1) promoter, a chondroitin sulfate proteoglycan (Cspg4) promoter, a CNP (2′,3′-Cyclic-nucleotide 3′-phosphodiesterase) promoter, and a GFAP promoter.

Also provided herein are pharmaceutical compositions comprising (a) a recombinant viral capsid as described herein and (b) a pharmaceutically acceptable carrier or excipient.

Such pharmaceutical compositions may be used to deliver a nucleotide of interest across a blood brain barrier in a mammalian subject. Such methods of delivering a nucleotide of interest across a blood brain barrier in a mammalian subject may comprise administering (e.g., contacting) the pharmaceutical composition to the mammal. In some embodiments, administering (e.g., contacting) is performed ex vivo. In some embodiments, administering is performed in a subject, optionally wherein the subject is modified to express the targeting ligand, e.g., from a safe harbor locus. In some embodiments, the subject is a primate animal, preferably a human. In some embodiments, the mammalian blood brain barrier cell is a mammalian brain endothelial cell. In some embodiments, endothelial cells in the mammalian blood brain barrier express transferrin receptor protein 1 on the cell surface and (i) the first member of the protein:protein binding pair, (ii) the second member of the protein:protein binding pair, and (iii) the antibody or binding portion thereof together direct the tropism of the viral vector to the endothelial cells in the mammalian blood brain barrier. In some embodiments, the viral particle is transported across the interior of the endothelial cells of the blood brain barrier for delivery to the brain by transcytosis after binding of the viral particle to the transferrin receptor protein 1 on the endothelial cell surface such that the endothelial cells are not infected by the viral particle. In some embodiments, the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule. In some embodiments, the nucleotide of interest is operably linked to a brain-specific promoter and the nucleotide of interest is preferentially expressed in the brain over other organs or tissues.

Also described herein is a method of treating a disease in a patient in need thereof comprising administering to the patient a viral particle or composition (e.g., pharmaceutical composition) described herein, wherein the viral particle comprises a nucleotide of interest encapsidated within the viral capsid, and wherein the nucleotide of interest encodes a therapeutic moiety, e.g., a therapeutic protein, a suicide gene, an antibody or a fragment thereof, a CRISPR/Cas system or a portion(s) thereof, an antisense oligonucleotide, a ribozyme, an RNAi molecule, or a shRNA molecule. In some embodiments, the therapeutic moiety targets, e.g., inhibits the action and/or expression of, α-synuclein. In some embodiments, the therapeutic moiety comprises a SNCA shRNA molecule, i.e., an shRNA molecule that targets, e.g., is complementary to, an mRNA molecule transcribed by a gene that encodes an α-synuclein protein, e.g., SNCA, for inhibition via RNA interference. In some embodiments, administration is via intravenous injection. In some embodiments, administration is via intracerebroventricular injection.

In some embodiments, the targeting ligand is operably linked to the protein (second member of a protein:protein binding pair), e.g., fused to the protein, optionally via a linker. In some embodiments, a targeting ligand may be a binding moiety, e.g., a natural ligand, antibody, a multispecific binding molecule, etc. In some embodiments, the targeting ligand is an antibody or portion thereof. In some embodiments, the targeting ligand is an antibody comprising a variable domain that binds TfR1 and a heavy chain constant domain. In some embodiments, the targeting ligand is an antibody comprising a variable domain that binds TfR1 on a target cell and an IgG heavy chain constant domain. In some embodiments, the targeting ligand is an antibody comprising a variable domain that binds TfR1 on a target cell and an IgG heavy chain constant domain, wherein the IgG heavy chain constant domain is operably linked, e.g., via a linker, to a protein (e.g., second member of a protein:protein binding pair) that forms an isopeptide covalent bond with the first member. In some embodiments, a capsid protein described herein comprises a first member comprising SpyTag operably linked to the viral capsid protein, and covalently linked to the SpyTag, and a second member comprising SpyCatcher linked to a targeting ligand comprising an antibody variable domain and an IgG heavy chain domain, wherein SpyCatcher and the IgG heavy chain domain are linked via an amino acid linker, e.g., GSGESG (SEQ ID NO:433).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows histograms obtained from flow cytometry analysis of green fluorescent protein (GFP) expression by cells expressing mTfR (“mTfR+293”; “bEnd.3 (mTfR+)”) or negative control cells that do not express mTfR but express hASGR1 (“hASGR1+293 Ts (ACL18620)”) after infection with AAV9 particles. Viruses were added at a multiplicity of infection of 1e5 vg/cell. Cells express GFP as a marker of transduction. Cells were transduced with a panel of AAV9 based particles including wildtype (wt) AAV9, or SpyTagged AAV9-based capsids (AAV9 wt, AAV9 N272A, AAV9 W503A) conjugated to an antibody 8D3 targeting mTfR (anti-TfR mAb), or conjugated to an antibody targeting hASGR1 as a negative control.

FIG. 2 shows immunohistochemistry staining for eGFP expression in the cerebellum or liver of wildtype C57BL/6J mice following injection of 5e10 vg/mouse of wildtype (wt) AAV9, or various AAV9-based capsids (AAV9 wt, AAV9 N272A, AAV9 W503A) conjugated to an antibody 8D3 targeting mTfR (anti-TfR mAb). Representative brain images depict coronal sections featuring a caudal plane that includes the cerebellum at 2.7× magnification. Liver sections are at 8× magnification.

FIG. 3 shows histograms obtained from flow cytometry evaluating green fluorescent protein (GFP) expression by cells expressing mTfR (mTfR+293 and bEnd.3) or cells expressing hASGR1 (hASGR1+293 (mTfR−)) as a negative control after infection with AAV9 particles. Viruses were added at a multiplicity of infection of 1e5 vg/cell. Cells express GFP as a marker of transduction. Cells were transduced with a panel of AAV9 based particles including wildtype (wt) AAV9, or SpyTagged AAV9 W503A particles conjugated to various formats of mTfR targeting antibody 8D3 either as bivalent antibody (mAb), Fab or scFv.

FIG. 4 shows immunohistochemistry staining for eGFP expression in the cerebellum and liver of WT C57BL/6J mice following injection of 7.5e9 vg/mouse (150 uL injection of 5e10 vg/mL solution) of wildtype (wt) AAV9, and de-targeted AAV9 W503A conjugated to antibodies targeting mTfR (anti-TfR mAb) in variety of antibody formats (bivalent “mAb”, Fab, and scFv). Representative brain images depict coronal sections featuring a caudal plane that includes the cerebellum at 4.4× magnification. Liver sections are at 14× magnification.

FIGS. 5A and 5B show qPCR data measuring viral DNA normalized to 3-actin and relative to wildtype (wt) AAV (y-axis) in the (FIG. 5A) brain and (FIG. 5B) liver of WT C57BL/6J mice following injection of 7.5e9 vg/mouse (150 uL injection of 5e10 vg/mL solution) of wildtype AAV9, de-targeted AAV9 W503A conjugated to antibodies targeting mTfR, or hASGR1 as a non-targeted control, in variety of antibody formats. AAV DNA was measured using a qPCR probe that recognizes the eGFP sequence and levels were normalized to a β-actin housekeeping gene. Mouse TfR-targeted AAV show enhanced levels of AAV DNA in brain 14 d post-injection relative to WT, detargeted, and hASGR1-targeted AAV9. Retargeting with TfR improves CNS transduction regardless of antibody format used.

FIG. 6 shows immunohistochemistry staining for eGFP expression in the brain of WT C57BL/6J mice following injection of wildtype (“WT”) AAV1, wildtype AAV1 conjugated to an antibody targeting mTfR (“8D3”), wildtype (“WT”) AAV8, or wildtype AAV8 conjugated to an antibody targeting mTfR (“8D3”, 2e12 vg/mouse), or wildtype AAV9 or wildtype AAV9 conjugated to an antibody targeting mTfR (“8D3”, 8e10 vg/mouse). Representative images include higher magnification coronal sections of planes featuring the hippocampus at 5× magnification, frontal cortex at 10× magnification, and cerebellum at 8× magnification. AAV particles targeted to human TFR specifically infect hTFR+ cell lines in vitro, and demonstrate enhanced CNS transduction and reduced liver transduction in vivo following systemic injection, regardless of AAV serotype.

FIG. 7 shows histograms obtained from flow cytometry evaluating green fluorescent protein (GFP) expression by cells expressing hTfR (hTfR+3T3) or cells not expressing hTfR (3T3) as a negative control after infection with AAV9 particles. Viruses were added at a multiplicity of infection of 1e5 vg/cell. Viruses express GFP as a marker of transduction. Cells were transduced with a panel of SpyTagged AAV9 W503A particles conjugated to Fabs targeting hTfR (Fabs H1H12799B, PN69331, H1H12848B, HIH31874B, H1H12843B, H1H12798B, H1H12850B, H1H12847B, H1H12835B, H1H12839B, H1H12841B, H1H12845B,) or to a Fab targeting hASGR1 as a negative control.

FIG. 8 shows immunohistochemistry staining for eGFP expression in the brain and liver of TFRC^{hu/hu} mice following injection of 1.5e10 vg/mouse (150 uL injection of 1e11 vg/mL solution) of wildtype (WT) AAV9, de-targeted AAV9 W503A conjugated to antibody Fabs targeting hTfR (H1H12845B), or antibody Fabs targeting hASGR1 as a non-targeted control. AAV9 wildtype particles can transduce the liver of TFRC^{hu/hu} mice, while AAV9 W503A particles are detargeted from the liver and do not facilitate high levels of liver eGFP expression. Representative images include higher magnification coronal sections of planes featuring the hippocampus at 9× magnification, frontal cortex at 10× magnification, medial cortex at 10× magnification, and cerebellum at 10× magnification.

FIG. 9A shows immunohistochemistry staining for eGFP expression in the brain of TFRC^{hu/hu} mice following injection of 1.5e10 vg/mouse (150 uL injection of 1e11 vg/mL solution) of wildtype (WT) AAV9, de-targeted AAV9 W503A conjugated to antibody Fabs targeting hTfR (Fabs H1H12845B, H1H12850B), or an antibody Fab targeting hASGR1 as a non-targeted control. Representative images include higher magnification coronal sections of planes featuring the hippocampus, frontal cortex, medial cortex, and cerebellum. FIG. 9B and FIG. 9C similarly show representative IHC staining for eGFP expression in the brain of TFRC^{hu/hu} mice injected with AAV9 W503A conjugated to additional Fabs targeting hTfR (FIG. 9B: H1H12841B, H1H12798B, H1H12847B, H1H12839B, H1H12843B; FIG. 9C: H1H31874B, H1H12835B, PN69331, H1H12848B, H1H12799B). Representative images include higher magnification coronal sections of planes featuring the hippocampus at 9× magnification, frontal cortex at 10× magnification, medial cortex at 10× magnification, and cerebellum at 10× magnification. FIG. 9D is a quantification of eGFP immunohistochemistry staining in brain (left panel) and liver (right panel) following injection of 1.5e10 vg/mouse of various AAV9 (x-axis): (1) wildtype AAV9, (2) de-targeted AAV9 W503A conjugated to antibody Fab targeting hASGR1 as a non-targeted control, or de-targeted AAV9 W503A conjugated to antibody Fabs targeting hTfR: (3) H1H12799B, (4) PN69331, (5) H1H12848B, (6) H1H31874B, (7) H1H12843B, (8) H1H12798B, (9) H1H12850B, (10) H1H12847B, (11) H1H12835B, (12) H1H12839B, (13) H1H12841B, and (14) H1H12845B. Percent area staining positive for DAB (indicates positive GFP signal) in brain and liver was quantified using HALO software.

FIG. 10A and FIG. 10B show qPCR data measuring AAV DNA in the (FIG. 10A) brain and (FIG. 10B) liver of TFRC^{hu/hu} mice following injection of (1) wildtype “WT: AAV9, (2) de-targeted AAV9 W503A conjugated to antibody Fabs targeting hASGR1 as a non-targeted control, or de-targeted AAV9 W503A conjugated to antibody Fabs targeting hTfR: (3) H1H12799B, (4) H1H12848B, (5) H1H31874B, (6) PN69331, (7) H1H12850B, (8) H1H12847B, (9) H1H12839B, (10) H1H12835B, (11) H1H12843B, (12) H1H12798B, (13) H1H12841B, or (14) H1H12845B. AAV DNA was measured using a qPCR probe targeting the eGFP sequence and levels were normalized to a β-actin housekeeping gene.

FIG. 11A provides heat maps that demonstrate enhanced transduction to brains and spinal columns in vivo in female humanized TFRC mice (TFRC^{hu/hu}) after administration of AAV9 particles, each with a unique barcode, and retargeted with anti-TfR Fabs, compared to wildtype AAV9 viral particles (AAV). Each candidate AAV was packaged with a unique barcoded genome as described in the Materials and Methods of the Examples below. Following IV dosing of the 36 candidate barcoded pool, the indicated tissues were collected and relative abundance of each barcode in the total RNA purified from each tissue was assessed using next generation sequencing (NGS). Shown are the enrichment values of each virus relative to the input viral pool in tissues of interest: liver (median lobe), left hemisphere of brain, and spinal column between cervical and sacrum. The data represented is the mean of three replicate animals in the study. FIG. 11B provides enrichment plots that demonstrate enhanced transduction to brains and spinal columns and liver detargeting in vivo for the mice in FIG. 11A.

FIG. 12A shows immunofluorescent co-staining for eGFP expression and brain cell-specific markers in the brain of TFRC^{hu/hu} mice following intravenous injection (1e11 vg/mouse) of de-targeted AAV9 W503A conjugated to antibody Fabs targeting hTfR (Fab H1H12845B). Brain transduction was assessed 19 days post-injection. Representative images include 20× magnification planes of brain sagittal sections featuring the cerebellum, cortex, and olfactory bulb. To characterize viral transgene expression by cell type, sections were co-stained for eGFP and cell-specific markers including microtubule-associated protein 2 (MAP2) and hexaribonucleotide binding protein 3 (Fox-3 or NeuN) for neurons; glial fibrillary acidic protein (GFAP) for astrocytes; 2′,3′-Cyclic-nucleotide 3′-phosphodiesterase (CNPase) and glutathione S-transferase π (GST-pi) for oligodendrocytes; cluster of differentiation 31 (CD31) for vascular endothelial cells and ionized calcium-binding adaptor molecule 1 (Iba1) for microglia. FIG. 12B shows immunofluorescent co-staining for eGFP expression and brain cell-specific markers in the brain of TFRC^{hu/hu} mice following intravenous injection (4e11 vg/mouse) of either WT AAV9 (top images) or de-targeted AAV9 W503A (bottom images) conjugated to antibody Fabs targeting hTfR (Fab H1H12845B) expressing H2B-eGFP fusion genome for nuclear expression of eGFP. Brain transduction was assessed 2 weeks post-injection. Representative images include 20× magnification planes of brain sagittal sections featuring the cortex, cerebellum, olfactory bulb and corpus callosum. To characterize viral transgene expression by cell type, sections were co-stained for eGFP and cell-specific markers including hexaribonucleotide binding protein 3 (Fox-3 or NeuN) and microtubule-associated protein 2 (MAP2) and for neurons; glial fibrillary acidic protein (GFAP) and SRY-box transcription factor 9 (Sox9) for astrocytes; 2′,3′-Cyclic-nucleotide 3′-phosphodiesterase (CNPase) and glutathione S-transferase π (GST-pi) for oligodendrocytes; cluster of differentiation 31 (CD31) for vascular endothelial cells; and ionized calcium-binding adaptor molecule 1 (Iba1) for microglia.

FIG. 13 shows immunohistochemistry staining for eGFP expression in the brain of TFRC^{hu/hu} mice following either intravenous (1e11 vg/mouse) or intracerebroventricular (1e10 vg/mouse) injection of wildtype (WT) AAV9, de-targeted AAV9 W503A conjugated to antibody Fabs targeting hTfR (Fab H1H12845B), or de-targeted AAV9 W503A conjugated to an antibody Fab targeting hASGR1 as a non-targeted control. Brain transduction was assessed 19 days post-injection. Representative images show whole brain sagittal sections.

FIG. 14A and FIG. 14B show qPCR data measuring viral DNA normalized to β-actin and relative to wildtype (WT) AAV9 (y-axis) in the brain (FIG. 14A) and liver (FIG. 14B) of TFRC^{hu/hu} mice following either intravenous (1e11 vg/mouse) or intracerebroventricular (1e10 vg/mouse) injection of WT AAV9, de-targeted AAV9 W503A conjugated to antibody Fabs targeting hTfR (Fab H1H12845B) or hASGR1 as a non-targeted control (x-axis). AAV DNA was measured using a qPCR probe that recognizes the eGFP sequence and levels were normalized to a β-actin housekeeping gene.

FIG. 15 shows immunohistochemistry staining for eGFP expression in the brain of TFRC^{hu/hu} mice following intravenous injection (1e11 vg/mouse) of wildtype (WT) AAV9, de-targeted AAV9 W503A conjugated to antibody Fabs targeting hTfR (Fabs H1H12845B, H1H12848B, H1H31874B, H1H12841B, H1H12839B, H1H12835B, H1H12847B, H1H12850B, H1H12798B, H1H12843B, PN69331, H1H12799B), or an antibody Fab targeting hASGR1 as a non-targeted control. Representative images include whole brain sagittal sections.

FIG. 16 shows the quantification of eGFP expression by immunohistochemistry staining across discrete brain regions of TFRC^{hu/hu} mice following intravenous injection (1e11 vg/mouse) of wildtype (WT) AAV9, de-targeted AAV9 W503A conjugated to antibody Fab targeting hASGR1 as a non-targeted control, or de-targeted AAV9 W503A conjugated to antibody Fabs targeting hTfR (H1H12845B, H1H12848B, H1H31874B, H1H12841B, H1H12839B, H1H12835B, H1H12847B, H1H12850B, H1H12798B, H1H12843B, PN69331 and H1H12799B). Percent area staining positive for DAB (y-axis, indicates positive eGFP signal) in brain was quantified using HALO software. Brain regions (x-axis) were defined and analyzed to include: (1) olfactory bulb, (2) cortex, (3) striatum, (4) hippocampus, (5) thalamus, (6) hypothalamus, (7) cerebellum, and (8) brainstem. AAV particles targeted to human TfR show enhanced brain transduction compared to WT AAV9 in multiple brain regions.

FIG. 17 shows the quantification of eGFP expression by immunohistochemistry staining in the cortex, hippocampus, thalamus, olfactory bulb, striatum, cerebellum, brainstem and hypothalamus of TFRC^{hu/hu} mice following intravenous injection of 1e11 vg/mouse of the following AAVs (x-axis): (1) wildtype (WT) AAV9, (2) de-targeted AAV9 W503A conjugated to antibody Fab targeting hASGR1 as a non-targeted control, or de-targeted AAV9 W503A conjugated to antibody Fabs targeting hTfR: (3) H1H12845B, (4) H1H12848B, (5) H1H31874B, (6) H1H12841B, (7) H1H12839B, (8) H1H12835B, (9) H1H12847B, (10) H1H12850B, (11) H1H12798B, (12) H1H12843B, (13) PN69331, and (14) H1H12799B. Percent area staining positive for DAB (y-axis, indicates positive eGFP signal) in brain was quantified using HALO software.

FIG. 18A and FIG. 18B show qPCR data measuring AAV DNA (y-axis) in the brain (FIG. 18A) and liver (FIG. 18B) of TFRC^{hu/hu} mice following intravenous injection 1e11 vg/mouse of (1) wildtype (WT) AAV9, (2) de-targeted AAV9 W503A conjugated to antibody Fab targeting hASGR1 as a non-targeted control or de-targeted AAV9 W503A conjugated to antibody Fabs targeting hTfR: (3) H1H12845B, (4) H1H12848B, (5) H1H31874B, (6) H1H12841B, (7) H1H12839B, (8) H1H12835B, (9) H1H12847B, (10) H1H12850B, (11) H1H12798B, (12) H1H12843B, (13) PN69331, and (14) H1H12799B, (x-axis). AAV DNA was measured using a qPCR probe targeting the eGFP sequence and levels were normalized to a GAPDH housekeeping gene and relative to wildtype WT AAV9.

FIG. 19A and FIG. 19B show qPCR data measuring AAV DNA (y-axis) in the brain (FIG. 19A) and liver (FIG. 19B) of WT C57BL/6J mice following intravenous injection of escalating doses (1.6e10, 8e10, 4e11 and 2e12 vg/mouse) of wildtype (WT) AAV9, WT AAV9 conjugated to antibody Fabs targeting mTfR (“8D3”), or de-targeted AAV9 W503A conjugated to antibody Fabs targeting mTfR (“8D3”) (x-axis). AAV DNA was measured using a qPCR probe targeting the eGFP sequence and levels were normalized to a R-actin housekeeping gene and relative to the highest dose of WT AAV9.

FIG. 20A and FIG. 20B show immunohistochemistry staining for eGFP expression in the brain of WT C57BL/6J mice following intravenous injection of escalating doses (1.6e10, 8e10, 4e11 and 2e12 vg/mouse) of wildtype (WT) AAV9, WT AAV9 conjugated to antibody Fabs targeting mTfR (“8D3”), or de-targeted AAV9 W503A conjugated to antibody Fabs targeting mTfR (“8D3”). Brain transduction was assessed 16 days post-injection. Representative images show low magnification of whole brain sagittal sections (FIG. 20A) as well as higher magnification planes from sagittal sections featuring the cortex, hippocampus, and cerebellum (FIG. 20B).

FIGS. 21A-21D show qPCR data measuring viral DNA normalized to β-actin and relative to wildtype (wt) AAV9 (y-axis) in the Liver (FIG. 21A), Heart (FIG. 21B), Quadriceps Femoris (FIG. 21C), and Brain (FIG. 21D) of female TFRC^{hu/hu} mice following injection of 1e11 vg/mouse of wildtype AAV9, de-targeted AAV9 W503A conjugated to antibodies and Fabs targeting hTfR, or hASGR1 as a non-targeted control. AAV DNA was measured using a qPCR probe that recognizes the eGFP sequence and levels were normalized to a β-actin housekeeping gene.

FIGS. 22A-22B show immunohistochemistry staining for eGFP expression in the brain, heart, quadriceps femoris, and liver of female TFRC^{hu/hu} mice following the injection of 1e11 vg/mouse of wildtype AAV9, and detargeted AAV9 W503A conjugated to H1H12845B Fab or H1H12845B mAb. Representative images include higher magnification brain sagittal sections of planes featuring (FIG. 22A) the frontal cortex at 12× magnification, vertical cross sections of the heart at 12× magnification, cross sections of quadriceps femoris at 12× magnification, and horizontal liver sections at 14× magnification; or (FIG. 22B) the cerebellum at 12× magnification (images in the first row from the top), hippocampus at 3× magnification (images in the second row from the top), cortex at 12× magnification (images in the third row from the top), and horizontal liver sections at 14× magnification (images in the fourth row from the top).

FIGS. 23A-23B show immunohistochemistry staining for eGFP expression in the brain, heart, quadriceps femoris, and liver of female TFRC^{hu/hu} mice following the injection of 1e11 vg/mouse of wildtype AAV9, and detargeted AAV9 W503A conjugated to H1H12839B Fab or H1H12839B mAb. Representative images include higher magnification brain sagittal sections of planes featuring (FIG. 23A) the frontal cortex at 12× magnification, vertical cross sections of the heart at 12× magnification, cross sections of quadriceps femoris at 12× magnification, and horizontal liver sections at 14× magnification; or (FIG. 23B) the cerebellum at 12× magnification, hippocampus at 3× magnification, cortex at 12× magnification, and horizontal liver sections at 14× magnification.

FIG. 24 demonstrates that AAV particles targeted to mouse TfR transduce neonatal spinal cord motor neurons when administered directly into the CNS. Representative immunofluorescence images of GFP expression in the spinal cord of P15 C57BL/6J mouse pups following a single intracerebroventricular (i.e.v.) injection at P0 of 1e11 vg of the indicated AAV viruses expressing GFP driven by the CBh promoter. Images include both coronal hemi-cord views of the lumbar spinal cord (top panel) as well as zoomed-in views of the ventral horn region (white box in top panel) where motor neurons reside, which are visualized using an antibody against the motor neuron marker choline acetyltransferse (ChAT). GFP expression was amplified with an antibody against GFP in these samples. Values plotted represent the mean and standard deviation of independent biological replicates.

FIG. 25 demonstrates that AAV particles targeted to mouse TfR highly transduce neonatal spinal cord motor neurons when administered intravenously. Representative immunofluorescence images of GFP expression in the spinal cord of P15 C57BL/6J mouse pups following a single intravenous (i.v.) injection into the facial vein of P0 mouse pups. Injection solution contained either no virus (i.e. PBS) or 5e11 vg of AAV9 WT retargeted to mTfR (8D3 fab) expressing GFP driven by the CBh promoter. Images include both coronal hemi-cord views of the lumbar spinal cord (top panel) as well as zoomed-in views of the ventral horn region (white box in top panel) where motor neurons reside, which are visualized using an antibody against the motor neuron marker choline acetyltransferse (ChAT). GFP signal shown here is native GFP expression without antibody amplification. Each panel represents independent biological replicates.

FIG. 26A shows qPCR data from the brain, spinal cord, liver, heart, quadriceps, and spleen of WT C57BL/6J mice measuring the abundance of antibody-encoding transcripts expressed by the indicated AAVs driven by the CAGG promoter following intravenous injection of 5×10¹¹ vg/mouse of de-targeted AAV9 W503A conjugated to 8D3 scFvs targeting mTfR or 5×10¹¹ vg/mouse of AAV8 (x-axis). AAV RNA was measured using a qPCR probe recognizing the expressed human antibody sequence, and relative mRNA levels were normalized to a β-actin housekeeping gene and relative to the AAV8 control group. FIG. 26B shows Enzyme Linked Immunosorbent Assay (ELISA) mediated detection of AAV-expressed human antibody titers in the brain lysates of WT C57BL/6J mice (y-axis) following intravenous injection of 5×10¹¹ vg/mouse of de-targeted AAV9 W503A conjugated to 8D3 scFvs targeting mTfR or 5e10 vg/mouse of AAV8. Antibody concentrations in brain were assessed 12 weeks post-injection.

FIG. 27 shows qPCR data measuring SNCA mRNA levels (y-axis) in the cortex, midbrain, and striatum of humanized SNCA mice following intravenous injection of 4×10¹¹ vg/mouse of de-targeted AAV9 W503A conjugated to 8D3 Fabs targeting mTfR and expressing SNCA shRNAs. SNCA mRNA was measured using a qPCR probe targeting the human SNCA sequence and SNCA mRNA levels were normalized to a GAPDH housekeeping gene and relative to SNCA mRNA level in the naïve (untreated) humanized SNCA mice group. SNCA mRNA levels in brain were assessed 1 month post-injection.

### DETAILED DESCRIPTION

### Transferrin Receptor

Transferrin (Tf) and its receptors (TfR) are central in the regulation of iron metabolism. There are two types of transferrin receptors: TfR1, also referred to as cluster of differentiation 71 (CD71), which is widely expressed and binds Tf with high affinity, and the less common TfR2, which is predominantly expressed in hepatocytes. “TfR” as used herein refers to TfR1 (CD71) unless specified otherwise.

The uptake of Tf-bound iron through TfR1 is the main source of cellular iron import in general. TfR1 is a 90 kDa type II transmembrane protein having 760 amino acids. It comprises a cytoplasmic N-terminal domain (amino acids 1-67), a transmembrane domain (amino acids 68-88), and a large extracellular C-terminal domain (amino acids 89-763), which comprises the Tf binding site. TfR1 may be generally found as a homodimer, with the monomers linked by disulfide bonds on the cell surface, with a molecular weight of about 180 kDa.

TfR is present both in human and non-human species, such as non-human primates and rodents. An exemplary amino acid sequence of human (h) TfR1 is set forth as SEQ ID NO:434, which is identical to the amino acid sequence of the hTfR1 protein represented as Uniprot P02786. The gene encoding for TfR, referred to as IFRC, is found on chromosome 3 in humans. An exemplary gene sequence for TFRC, with annotated exons and introns, can be found from the NCBI database (Gene ID: 7037).

An exemplary amino acid sequence of mouse (m) TfR1 is set forth as SEQ ID NO:435, which is identical to the amino acid sequence of the mTfR1 protein represented as Uniprot Q62351 and which has about 77% amino acid sequence identity with hTfR1. The Tfrc gene is found on chromosome 16 in mice. The complete gene sequence for mouse Tfrc, with annotated exons and introns, can be found from the NCBI database (Gene ID: 22042).

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The “percent (%) identity” or the like may be readily determined for amino acid or nucleotide sequences, over the full-length of a protein, or a portion thereof. A portion may be at least about 5 amino acids or 24 nucleotides, respectively, in length, and may be up to about 700 amino acids or 2100 nucleotides, respectively. Generally, when referring to “identity” between HCVR and LCVRs, the percent identity refers to the identity spanning length of the HCDR1, HCDR2, HCDR3 and LCDR1, LCDR2, and LCDR3. Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments may be performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using FASTA™, a program in GCG Version 6.1. Fasta™ provides alignments and percent sequence identity of the regions of the best overlap between the query and search sequences. For instance, percent sequence identity between nucleic acid sequences can be determined using FASTA™ with its default parameters (a word size of 6 and the NOPAM factor for the scoring matrix) as provided in GCG Version 6.1, herein incorporated by reference.

“Significant identity” encompasses amino acid or nucleic acid sequences alignments that are at least 90%, e.g., at least 93%, e.g., at least 95%, e.g., at least 96%, e.g., at least 97%, e.g., at least 98%, e.g., at least 99%, or e.g., at least 100% identical.

The term “chimeric” encompasses a functional gene or polypeptide comprising nucleic acid sequences or amino acid sequences, respectively, from at least two different organisms, e.g., portions of a gene or polypeptide of at least a first and second AAV, wherein the at least first and second portions are operably linked. Unless specified as chimeric, nucleotide sequences, genes, polypeptides, and amino acids are considered non-chimeric, e.g., comprising a nucleic acid sequence or amino acid sequence of only a single organism, e.g., a single AAV.

The term “antibody” includes immunoglobulin molecules comprising four polypeptide chains, two heavy (H) chains and two light (L) chains inter-connected by disulfide bonds. Each heavy chain comprises a heavy chain variable domain (V_{H}) and a heavy chain constant region (C_{H}). The heavy chain constant region comprises at least three domains, C_{H}1, C_{H}2, C_{H}3 and optionally C_{H}4. Each light chain comprises a light chain variable domain (C_{H}) and a light chain constant region (C_{L}). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. Typical tetrameric antibody structures comprise two identical antigen-binding domains, each of which formed by association of the V_{H} and V_{L} domains, and each of which together with respective C_{H} and C_{L} domains form the antibody Fv region. Single domain antibodies comprise a single antigen-binding domain, e.g., a V_{H} or a V_{L}. The antigen-binding domain of an antibody, e.g., the part of an antibody that recognizes and binds to the first member of a specific binding pair of an antigen, is also referred to as a “paratope.” It is a small region (of 5 to 10 amino acids) of an antibody's Fv region, part of the fragment antigen-binding (Fab region), and may contain parts of the antibody's heavy and/or light chains. The term “single-chain variable fragment” or “scFv” includes a single chain fusion polypeptide containing an immunoglobulin heavy chain variable region (VH) and an immunoglobulin light chain variable region (VL). In some embodiments, the VH and VL are connect by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. Ahmad et al., Clinical and Developmental Immunology, volume 2012, article ID 98025 is incorporated herein by reference for methods of making scFv fragments by phage display and antibody domain cloning. A paratope specifically binds a first member of a specific binding pair when the paratope binds the first member of a specific binding pair with a high affinity. The term “high affinity” antibody refers to an antibody that has a K_{D} with respect to its target first member of a specific binding pair about of 10⁻⁹ M or lower (e.g., about 1×10⁻⁹ M, 1×10⁻¹⁰ M, 1×10⁻⁴ M, or about 1×10⁻¹² M). In one embodiment, K_{D} is measured by surface plasmon resonance, e.g., BIACORE™; in another embodiment, K_{D} is measured by ELISA.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germ line sequence or a rearranged or unrearranged sequence, and, for example, by a naive or a mature B cell or a T cell. A CDR can be somatically mutated (e.g., vary from a sequence encoded in an animal's germ line), humanized, and/or modified with amino acid substitutions, additions, or deletions. In some circumstances (e.g., for a CDR3), CDRs can be encoded by two or more sequences (e.g., germ line sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “light chain” includes an immunoglobulin light chain sequence from any organism, and unless otherwise specified includes human κ and λ light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises V_{L} and J_{L} segments, derived from a repertoire of V and J segments present in the germ line. Sequences, locations and nomenclature for V and J light chain segments for various organisms can be found in IMGT database, www.imgt.org. Light chains include those, e.g., that do not selectively bind either a first or a second first member of a specific binding pair selectively bound by the first member of a specific binding pair-binding protein in which they appear. Light chains also include those that bind and recognize or assist the heavy chain or another light chain with binding and recognizing, one or more first member of a specific binding pairs selectively bound by the first member of a specific binding pair-binding protein in which they appear. Common or universal light chains include those derived from a human Vκ1-39Jκ gene or a human Vκ3-20Jκ gene, and include somatically mutated (e.g., affinity matured) versions of the same. Exemplary human V_{L} segments include a human Vκ1-39 gene segment, a human Vκ3-20 gene segment, a human Vλ1-40 gene segment, a human Vλ1-44 gene segment, a human Vλ2-8 gene segment, a human Vλ2-14 gene segment, and human Vλ3-21 gene segment, and include somatically mutated (e.g., affinity matured) versions of the same. Light chains can be made that comprise a variable domain from one organism (e.g., human or rodent, e.g., rat or mouse; or bird, e.g., chicken) and a constant region from the same or a different organism (e.g., human or rodent, e.g., rat or mouse; or bird, e.g., chicken).

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an first member of a specific binding pair (e.g., recognizing the first member of a specific binding pair with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “heavy chain only antibody,” ““heavy chain only antigen binding protein,” “single domain antigen binding protein,” “single domain binding protein” or the like refers to a monomeric or homodimeric immunoglobulin molecule comprising an immunoglobulin-like chain comprising a variable domain operably linked to a heavy chain constant region, that is unable to associate with a light chain because the heavy chain constant region typically lacks a functional C_{H}1 domain. Accordingly, the term “heavy chain only antibody,” “heavy chain only antigen binding protein,” “single domain antigen binding protein,” “single domain binding protein” or the like encompasses a both (i) a monomeric single domain antigen binding protein comprising one of the immunoglobulin-like chain comprising a variable domain operably linked to a heavy chain constant region lacking a functional C_{H}1 domain, or (ii) a homodimeric single domain antigen binding protein comprising two immunoglobulin-like chains, each of which comprising a variable domain operably linked to a heavy chain constant region lacking a functional C_{H}1 domain. In various aspects, a homodimeric single domain antigen binding protein comprises two identical immunoglobulin-like chains, each of which comprising an identical variable domain operably linked to an identical heavy chain constant region lacking a functional C_{H}1 domain. Additionally, each immunoglobulin-like chain of a single domain antigen binding protein comprises a variable domain, which may be derived from heavy chain variable region gene segments (e.g., V_{H}, D_{H}, J_{H}), light chain gene segments (e.g., V_{L}, J_{L}), or a combination thereof, linked to a heavy chain constant region (C_{H}) gene sequence comprising a deletion or inactivating mutation in a C_{H}1 encoding sequence (and, optionally, a hinge region) of a heavy chain constant region gene, e.g., IgG, IgA, IgE, IgD, or a combination thereof. A single domain antigen binding protein comprising a variable domain derived from heavy chain gene segments may be referred to as a “V_{H}-single domain antibody” or “V_{H}-single domain antigen binding protein”, see, e.g., U.S. Pat. No. 8,754,287; U.S. Patent Publication Nos. 20140289876; 20150197553; 20150197554; 20150197555; 20150196015; 20150197556 and 20150197557, each of which is incorporated in its entirety by reference. A single domain antigen binding protein comprising a variable domain derived from light chain gene segments may be referred to as a or “V_{L}-single domain antigen binding protein,” see, e.g., U.S. Publication No. 20150289489, incorporated in its entirety by reference.

The phrase “light chain” includes an immunoglobulin light chain sequence from any organism, and unless otherwise specified includes human kappa (κ) and lambda (λ) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IMGT database, which is accessible via the internet on the world wide web (www) at the URL “imgt.org.” Light chains include those, e.g., that do not selectively bind either a first or a second first member of a specific binding pair selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Common or universal light chains include those derived from a human Vκ1-39Jκ5 gene or a human Vκ3-20Jκ1 gene, and include somatically mutated (e.g., affinity matured) versions of the same.

The phrase “operably linked”, as used herein, includes a physical juxtaposition (e.g., in three-dimensional space) of components or elements that interact, directly or indirectly with one another, or otherwise coordinate with each other to participate in a biological event, which juxtaposition achieves or permits such interaction and/or coordination. To give but one example, a control sequence (e.g., an expression control sequence) in a nucleic acid is said to be “operably linked” to a coding sequence when it is located relative to the coding sequence such that its presence or absence impacts expression and/or activity of the coding sequence. In many embodiments, “operable linkage” involves covalent linkage of relevant components or elements with one another. Those skilled in the art will readily appreciate that, in some embodiments, covalent linkage is not required to achieve effective operable linkage. For example, in some embodiments, nucleic acid control sequences that are operably linked with coding sequences that they control are contiguous with the nucleotide of interest. Alternatively or additionally, in some embodiments, one or more such control sequences acts in trans or at a distance to control a coding sequence of interest. In some embodiments, the term “expression control sequence” as used herein refers to polynucleotide sequences which are necessary and/or sufficient to effect the expression and processing of coding sequences to which they are ligated. In some embodiments, expression control sequences may be or comprise appropriate transcription initiation, termination, promoter and/or enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and/or, in some embodiments, sequences that enhance protein secretion. In some embodiments, one or more control sequences are preferentially or exclusively active in a particular host cell or organism, or type thereof. To give but one example, in prokaryotes, control sequences typically include promoter, ribosomal binding site, and transcription termination sequence; in eukaryotes, in many embodiments, control sequences typically include promoters, enhancers, and/or transcription termination sequences. Those of ordinary skill in the art will appreciate from context that, in many embodiments, the term “control sequences” refers to components whose presence is essential for expression and processing, and in some embodiments includes components whose presence is advantageous for expression (including, for example, leader sequences, targeting sequences, and/or fusion partner sequences).

“Retargeting” or “redirecting” may include a scenario in which the wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

“Specific binding pair,” “protein:protein binding pair” and the like includes two proteins (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope) or a covalent isopeptide bond under conditions that enable or facilitate bond formation. In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002; SpyTag:KTag; isopeptag:pilin C, SnoopTag:SnoopCatcher, etc. Generally, a first member of a protein:protein binding pair refers to member of a protein:protein binding pair, which is generally less than 30 amino acids in length, and which forms a covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e., between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 and Zaveri et al. (2012) PNAS 109:E690-E697, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO:321) which forms an amide bond to its cognate protein “SpyCatcher,” an 112 amino acid polypeptide having the amino acid sequence set forth in SEQ ID NO:322. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181). SpyLigase (SEQ ID NO: 389) was engineered by excising the 3 strand from SpyCatcher that contains a reactive lysine, resulting in KTag, 10-residue first member of a protein:protein binding pair having the amino acid sequence ATHIKFSKRD (SEQ ID NO:323). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK, set forth as SEQ ID NO:324, and binds SpyCatcher002 (SEQ ID NO:442).

The SnoopTag:SnoopCatcher system is described in Veggiani (2016) PNAS 113:1202-07. The D4 Ig-like domain of RrgA, an adhesin from Streptococcus pneumoniae, was split to form SnoopTag (residues 734-745; SEQ ID NO:390) and SnoopCatcher (residues 749-860; SEQ ID NO:391). Incubation of SnoopTag and SnoopCatcher results in a spontaneous isopeptide bond that is specific between the complementary proteins. Veggiani (2016)), supra.

The isopeptag:pilinC specific binding pair was derived from the major pilin protein Spy0128 from Streptococcuspyogenes. (Zakeir and Howarth (2010) J. Am. Chem. Soc. 132:4526-27). Isopeptag has the amino acid sequence TDKDMTITFTNKKDAE, set forth as SEQ ID NO:325, and binds pilin-C (residues 18-299 of Spy0128). Incubation of isopeptag and pilinC results in a spontaneous isopeptide bond that is specific between the complementary proteins. Zakeir and Howarth (2010), supra.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag, FLAG tag, Strep II tag, streptavidin-binding peptide (SBP) tag, calmodulin-binding peptide (CBP), glutathione S-transferase (GST), maltose-binding protein (MBP), S-tag, HA tag, and c-myc (SEQ ID NO:326). (Reviewed in Zhao et al. (2013) J. Analytical Meth. Chem. 1-8; incorporated herein by reference). A common detectable label for primate AAV is the B1 epitope (SEQ ID NO:327). Some AAV capsid proteins described herein, which do not naturally comprise the B1 epitope, may be modified herein to comprise a B1 epitope. Generally, AAV capsid proteins described herein may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

The term “target cells” includes any cells in which expression of a nucleotide of interest is desired. Preferably, target cells exhibit a receptor on their surface that allows the cell to be targeted with a targeting ligand, as described below.

The term “transduction” or “infection” or the like refers to the introduction of a nucleic acid into a target cell nucleus by a viral particle. The term efficiency in relation to transduction or the like, e.g., “transduction efficiency” refers to the fraction (e.g., percentage) of cells expressing a nucleotide of interest after incubation with a set number of viral particles comprising the nucleotide of interest. Well-known methods of determining transduction efficiency include flow cytometry of cells transduced with a fluorescent reporter gene, RT-PCR for expression of the nucleotide of interest, etc.

Generally, “reference” viral capsid protein/capsid/particle are identical to test viral capsid protein/capsid/particle but for the change for which the effect is to be tested. For example, to determine the effect, e.g., on transduction efficiency, of inserting a first member of a specific binding pair into a test viral particle, the transduction efficiencies of the test viral particle (in the absence or presence of an appropriate targeting ligand) can be compared to the transduction efficiencies of a reference viral particle (in the absence or presence of an appropriate targeting ligand if necessary) which is identical to the test viral particle in every instance (e.g., additional point mutations, nucleotide of interest, numbers of viral particles and target cells, etc.) except for the presence of a first member of a specific binding pair. In some embodiments, a reference viral capsid protein is one that is able to form a capsid with a second viral capsid protein modified to comprise at least a first member of a protein:protein binding pair, where the reference viral capsid protein does not comprise the first member of a protein:protein binding pair, preferably wherein the capsid formed by the reference viral capsid protein and the modified viral capsid protein is a mosaic capsid.

### Adeno-Associated Viruses (AAV)

“AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 5 herein provides sequences of various non-primate AAV.

“AAV” encompasses all subtypes and both naturally occurring and modified forms, except where stated otherwise. AAV includes primate AAV (e.g., AAV type 1 (AAV1), primate AAV type 2 (AAV2), primate AAV type 3 (AAV3), primate AAV3B, primate AAV type 4 (AAV4), primate AAV type 5 (AAV5), primate AAV type 6 (AAV6), primate AAV6.2, primate AAV type 7 (AAV7), primate AAV type 8 (AAV8), primate AAV type 9 (AAV9), AAV10, AAV type hu11 (AAV hu11), AAV11, AAV12, AAV13, AAVDJ, Anc80L65, AAV2G9, AAVLK03, AAV type rh32.33 (AAVrh.32.33), AAV retro (AAV retro), AAV PHP.B, AAV PHP.eB, AAV PHP.S, AAVrh.64R1, AAVhu.37, AAVrh.8, AAV2/8, etc.; non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc., “Primate AAV” refers to AAV generally isolated from primates. Similarly, “non-primate animal AAV” refers to AAV isolated from non-primate animals.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA2 region, VP1-u region, VP1/VP2 common region, VP3 region), nucleotide sequence (e.g., ITR sequence), e.g., a cap gene or capsid protein of AAV etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional Cap protein.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein” and the like includes a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA2), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional ~60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions provided herein may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

Non-limiting examples of wildtype and/or genetically modified nucleic acid sequences of cap genes and cap proteins useful for retargeting viral particles as described herein are set forth in SEQ ID NOs: 392-432.

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A “D” region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep 52 and/or Rep40 Rep proteins. Alternatively, Rep proteins may be expressed individually from an ORF comprising a nucleotide sequence encoding any one of Rep78, Rep68, Rep 52, or Rep40 by use of separate nucleotide sequences operably linked to one expression control sequence for expression in a packaging cell, each producing only one Rep78, Rep68, Rep 52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid. However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

The term “portion” refers to at least 5 amino acids or at least 15 nucleotides, but less than the full-length polypeptide or nucleic acid molecule, with 100% identity to a sequence from which the portion is derived, see Penzes (2015) J. General Virol. 2769. A “portion” encompasses any contiguous segment of amino acids or nucleotides sufficient to determine that the polypeptide or nucleic acid molecule form which the portion is derived is “of a [specified] AAV” or has “significant identity” to a particular AAV, e.g., a non-primate animal AAV or remote AAV. In some embodiments, a portion comprises at least 5 amino acids or 15 nucleotides with 100% identity to a sequence associated with the specified AAV. In some embodiments, a portion comprises at least 10 amino acids or 30 nucleotides with 100% identity to a sequence associated with the specified AAV. In some embodiments, a portion comprises at least 15 amino acids or 45 nucleotides with 100% identity to a sequence associated with the specified AAV. In some embodiments, a portion comprises at least 20 amino acids or 60 nucleotides with 100% identity to a sequence associated with the specified AAV. In some embodiments, a portion comprises at least 25 amino acids or 75 nucleotides with 100% identity to a sequence associated with the specified AAV. In some embodiments, a portion comprises at least 30 amino acids or 90 nucleotides with 100% identity to a sequence associated with the specified AAV. In some embodiments, a portion comprises at least 35 amino acids or 105 nucleotides with 100% identity to a sequence associated with the specified AAV. In some embodiments, a portion comprises at least 40 amino acids or 120 nucleotides with 100% identity to a sequence associated with the specified AAV. In some embodiments, a portion comprises at least 45 amino acids or 135 nucleotides with 100% identity to a sequence associated with the specified AAV. In some embodiments, a portion comprises at least 50 amino acids or 150 nucleotides with 100% identity to a sequence associated with the specified AAV. In some embodiments, a portion comprises at least 60 amino acids or 180 nucleotides with 100% identity to a sequence associated with the specified AAV. In some embodiments, a portion comprises at least 70 amino acids or 210 nucleotides with 100% identity to a sequence associated with the specified AAV. In some embodiments, a portion comprises at least 80 amino acids or 240 nucleotides with 100% identity to a sequence associated with the specified AAV. In some embodiments, a portion comprises at least 90 amino acids or 270 nucleotides with 100% identity to a sequence associated with the specified AAV. In some embodiments, a portion comprises at least 100 amino acids or 300 nucleotides with 100% identity to a sequence associated with the specified AAV.

### Modified Virus Capsid Proteins, Viral Particles, Nucleic Acids

In some embodiments, a Cap protein, e.g., a VP1 capsid protein as described herein, a VP2 capsid protein as described herein, and/or a VP3 capsid protein as described herein, is modified to comprise e.g., a first member of a protein:protein binding pair, a detectable label, point mutation, etc.

Chimerism is a type of modification as described herein. Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide. A modification may include an insertion of, e.g., a first member of a protein:protein binding pair and a point mutation, e.g., such that the natural tropism of the capsid protein is reduced to abolished and/or such that the capsid protein comprises a detectable label. Preferred modifications include those that do not alter and preferably decrease the low to no recognition of the modified capsid by pre-existing antibodies found in the general population that were produced during the course of infection with another AAV, e.g., infection with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof. Other modifications as described herein include modification of a capsid protein such that it comprises a first member of a protein:protein binding pair, a detectable label, etc., which modifications generally result from modifications at the genetic level, e.g., via modification of a cap gene.

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a protein:protein binding pair and a second corresponding viral capsid protein lacking the first member of a protein:protein binding pair. In relation to a mosaic capsid, the second viral capsid protein lacking the first member of a protein:protein binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a protein:protein binding pair, except that the reference capsid protein lacks the first member of a protein:protein binding pair. In some mosaic capsid embodiments, a VP1, VP2, and/or VP3 reference capsid protein corresponds to the viral VP1, VP2, and/or VP3 capsid protein modified with a first member of a protein:protein binding pair, except that the reference capsid protein lacks the first member of a protein:protein binding pair. In some embodiments, a VP1 reference capsid protein corresponds to the viral VP1 capsid protein modified with a first member of a protein:protein binding pair, except that the reference capsid protein lacks the first member of a protein:protein binding pair. In some embodiments, a VP2 reference capsid protein corresponds to the viral VP2 capsid protein modified with a first member of a protein:protein binding pair, except that the reference capsid protein lacks the first member of a protein:protein binding pair. In some embodiments, a VP3 reference capsid protein corresponds to the viral VP3 capsid protein modified with a first member of a protein:protein binding pair, except that the reference capsid protein lacks the first member of a protein:protein binding pair. In some mosaic capsid embodiments comprising a chimeric VP1, VP2, and/or VP3 capsid protein further modified to comprise a first member of a protein:protein binding pair, a reference protein may be a corresponding capsid protein from which portions thereof form part of the chimeric capsid protein. As a non-limiting example in some embodiments, a mosaic capsid comprising a chimeric AAV2/AAAV VP1 capsid protein modified to comprise a first member of a protein:protein binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the protein:protein binding pair and is able to form a capsid with the first capsid protein modified with the first member of a protein:protein binding pair.

Generally, mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a protein:protein binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a protein:protein binding pair. In some non-mosaic viral particle embodiments, the protein subunit ratio may be 0:1 wherein each capsid protein of the non-mosaic viral particle is not modified with a first member of a protein:protein binding pair.

In some embodiments, a capsid protein of the invention is modified to comprise a detectable label. Many detectable labels are known in the art. (See, e.g.: Nilsson et al. (1997) “Affinity fusion strategies for detection, purification, and immobilization of modified proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6, His-8, or His-10) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag, commercially available from Covalys (www.covalys.com). In some embodiments, a detectable label disclosed herein comprises a detectable label recognized only by an antibody paratope. In some embodiments, a detectable label disclosed herein comprises a detectable label recognized by an antibody paratope and other specific binding pairs.

In some embodiments, the detectable label forms a binding pair with an immunoglobulin constant domain. In some embodiments, the detectable label and/or detectable label does form a binding pair with a metal ion, e.g., Ni²⁺, Co²⁺, Cu²⁺, Zn²⁺, Fe³⁺, etc. In some embodiments, the detectable label is selected from the group consisting of Streptavidin, Strep II, HA, L14, 4C-RGD, LH, and Protein A.

In some embodiments, the detectable label is selected from the group consisting of FLAG, HA and c-myc (EQKLISEEDL; SEQ ID NO:326). In some embodiments, the detectable label is c-myc (SEQ ID NO:326).

In some embodiments, a detectable label is a B cell epitope, e.g., is between about 1 amino acid and about 35 amino acids in length, and forms a binding pair with an antibody paratope, e.g., an immunoglobulin variable domain. In some embodiments, the detectable label comprises a B1 epitope (SEQ ID NO:327). In some embodiments, a capsid protein is modified to comprise a B1 epitope in the VP3 region.

In some embodiments, a capsid protein of the invention comprises at least a first member of a peptide:peptide binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc (SEQ ID NO:326). Use of a detectable label as a first member of a protein:protein binding pair is described in, e.g., WO2019006043, incorporated herein in its entirety by reference.

In some embodiments, a capsid protein comprises a first member of a protein:protein binding pair, wherein the protein:protein binding pair forms a covalent isopeptide bond. In some embodiments, the first member of a protein:protein binding pair is covalently bound via an isopeptide bond to a cognate second member of the protein:protein binding pair, and optionally wherein the cognate second member of the protein:protein binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag:KTag, Isopeptag:pilinC, and SnoopTag:SnoopCatcher. In some embodiments, the first member is SpyTag (or a biologically active portion thereof) and the protein (second cognate member) is SpyCatcher (or a biologically active portion thereof). In some embodiments, the first member is SpyTag (or a biologically active portion thereof) and the protein (second cognate member) is KTag (or a biologically active portion thereof). In some embodiments, the first member is KTag (or a biologically active portion thereof) and the protein (second cognate member) is SpyTag (or a biologically active portion thereof). In some embodiments, the first member is SnoopTag (or a biologically active portion thereof) and the protein (second cognate member) is SnoopCatcher (or a biologically active portion thereof). In some embodiments, the first member is Isopeptag (or a biologically active portion thereof) and the protein (second cognate member) is Pilin-C (or a biologically active portion thereof). In some embodiments, the first member is SpyTag002 (or a biologically active portion thereof) and the protein (second cognate member) is SpyCatcher002 (or a biologically active portion thereof). In some embodiments, a Cap protein of the invention comprises a SpyTag. Use of a first member of a protein:protein binding pair is described in WO2019006046, incorporated herein in its entirety.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length, e.g., the linker is GLSG (SEQ ID NO:328). In some embodiments, the first and second linkers are identical in length, and are each five amino acids in length. In some embodiments, the first and second linkers are identical in length, and are each six amino acids in length, e.g., the first and second linkers each comprise a sequence of GLSGSG (SEQ ID NO:329). In some embodiments, the first and second linkers are identical in length, and are each seven amino acids in length. In some embodiments, the first and second linkers are identical in length, and are each eight amino acids in length, e.g., the first and second linkers each comprise a sequence of GLSGLSGS (SEQ ID NO:330). In some embodiments, the first and second linkers are identical in length, and are each nine amino acids in length. In some embodiments, the first and second linkers are identical in length, and are each ten amino acids in length, e.g., the first and second linkers each comprise a sequence of GLSGLSGLSG (SEQ ID NO:331) or GLSGGSGLSG (SEQ ID NO:332). In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids to about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

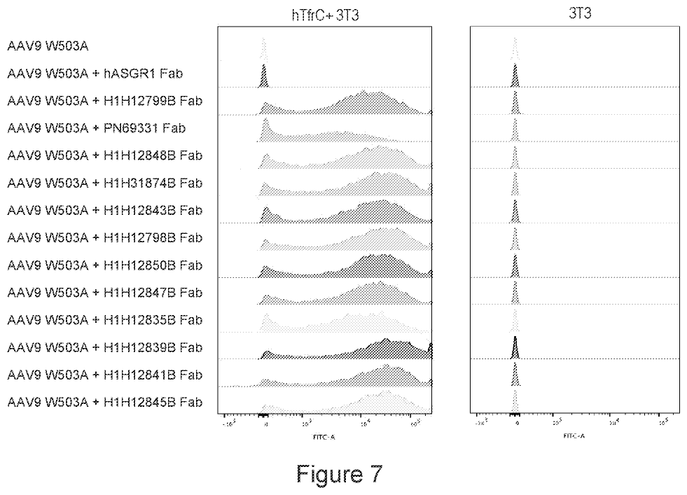

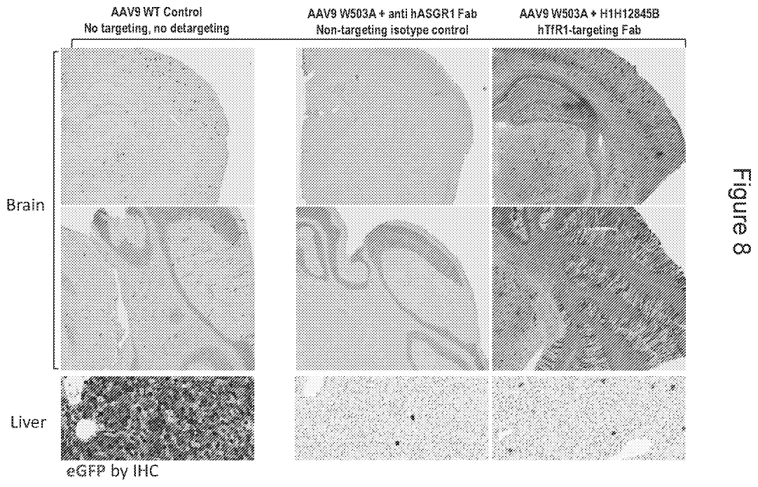

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of a first member of a protein:protein binding pair and/or detectable label.

Accordingly, in some embodiments, the first member of a protein:protein binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a protein:protein binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, 1261, I-266, I-381, I-453, I-447, I-448, I-459, I-471, I-520, I-534, I-570, I-573, I-584, I-587, I-588, I591, I-657, I-664, I-713 and I-716 of the VP1 capsid protein of AAV2 (Wu et al. (2000) J. Virol. 74:8635-8647). In some embodiments, an insertion site of a non-primate animal VP1 capsid protein corresponds to I-453. A modified virus capsid protein as described herein may be a non-primate animal capsid protein comprising a first member of a protein:protein binding pair and/or detectable label inserted into a position corresponding with a position of an AAV2 capsid protein selected from the group consisting of I-1, I-34, I-138, I-139, I-161, 1261, I-266, I-381, I-447, I-448, I-453, I-459, I-471, I-520, I-534, I-570, I-573, I-584, I-587, I-588, I591, I-657, I-664, I-713, I-716, and a combination thereof. In some embodiments, an insertion site of a non-primate animal VP1 capsid protein corresponds to I-453. Additional suitable insertion sites of a non-primate animal AAV that include those corresponding to I-587 of AAV1, I-589 of AAV1, I-585 of AAV3, I-585 of AAV4, and I-585 of AAV5. In some embodiments, a modified virus capsid protein as described herein may be a non-primate animal capsid protein comprising a first member of a protein:protein binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a protein:protein binding pair and/or detectable label is inserted in a VP1 capsid protein of a non-primate animal AAV after an amino acid position corresponding with an amino acid position selected from the group consisting of I444 of an avian AAV capsid protein VP1, I580 of an avian AAV capsid protein VP1, I573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and I565 of a sea lion AAV capsid protein VP1.

The nomenclature I-###, I # or the like herein refers to the insertion site (I) with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 1.5-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a protein:protein binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a protein:protein binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC50 value that is at least 2-fold that of a control virus particle).

### Targeting Ligands

A viral particle described herein may further comprise a targeting ligand. “Retargeting” or “directing” may include a scenario in which the wildtype viral particle targets several cells within a tissue and/or several organs within an organism, which broad targeting of the tissue or organs is reduced to abolished by insertion of a detectable label or a targeting ligand, and which retargeting to more specific cells in the tissue or more specific organ in the organism is achieved, respectively with a multispecific binding molecule that binds the detectable label and a second domain that binds a receptor of interest and/or with a targeting ligand that binds the receptor of interest. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule.

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule includes those binding molecules comprising (i) an antibody paratope that specifically binds the detectable label and (ii) a second binding domain that specifically binds a receptor targets the viral particle.

In some embodiments of the invention, a viral vector comprises a protein:protein binding pair associated by an isopeptide bond as described herein, wherein the second member of the protein:protein binding pair is fused to a targeting ligand. In some embodiments, a targeting ligand fused to a second member of a protein:protein binding pair associated by an isopeptide bond comprises an antibody, or binding portion thereof, e.g., an antibody paratope.

An antibody paratope as described herein generally comprises at a minimum a complementarity determining region (CDR) that is involved in the specific recognition of a target (e.g., a detectable label, a cell surface receptor, etc.) e.g., a CDR3 region of a heavy and/or light chain variable domain. In some embodiments, a multispecific binding molecule comprises an antibody (or portion thereof) that comprises the antibody paratope that specifically binds the detectable label.

One embodiment of the present invention is a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a highly preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

Generally, viral capsid proteins as described herein may comprise a targeting ligand that targets TfR, such as anti-TfR-antibodies and binding portions thereof. Antibodies that specifically human TfR are well-known in the art. For non-limiting exemplary anti-transferrin receptor antibodies; see, e.g., see, e.g., US20170174778; US20150196663; U.S. Pat. No. 9,629,801; US20180002433; WO2016081643; US20180134797; WO2014189973; US20150110791; U.S. Pat. No. 9,708,406; US20170260292; WO2016081640; US20180057604; U.S. Pat. No. 9,611,323; WO2012075037; WO2018210898, US20180344869, US20180282408, US20170051071, WO2016207240, WO2015101588, US20160324984; US20180222993; WO2017055542; US20180222992; WO2017055540; Cabezon, I., et al. Mol Pharm. 2015 Nov. 2; 12(11):4137-45; Yu Y J, et al. Sci Transl Med (2014) 6:261ra154; Couch, et al. Sci Transl Med. 2013 May 1; 5(183):183ra57, 1-12.

Additional nucleic acid sequences and translated amino acid sequences of domains of anti-transferrin antibodies and scFvs that may be used to retarget AAV capsids as described herein are provided as SEq ID NOs:1-320 and 333-388.

Table 1 provides a summary of the SEQ ID NO for each binding portion (e.g., heavy chain variable domain, light chain variable domain, and CDR1, CDR2, and CDR3) of non-limiting anti-human-TfR antibodies that may be used to redirect an AAV capsid as described herein. In some embodiments, an AAV capsid as described herein comprises a targeting ligand that binds human TfR, wherein the targeting ligand comprises a heavy chain variable domain, light chain variable domain, CDR1, CDR2, and/or CDR3 amino acid sequence at least 90% identical to, respectively, an amino acid sequence of a heavy chain variable domain, light chain variable domain, CDR1, CDR2, and/or CDR3 as set forth in any one of SEQ ID NOs:1-320 and 365-388. In some embodiments, an AAV capsid as described herein comprises a targeting ligand that binds human TfR, wherein the targeting ligand comprises a heavy chain variable domain, light chain variable domain, CDR1, CDR2, and/or CDR3 amino acid sequence at least 95% identical to, respectively, an amino acid sequence of a heavy chain variable domain, light chain variable domain, CDR1, CDR2, and/or CDR3 set forth in any one of SEQ ID NOs:1-320 and 365-388. In some embodiments, an AAV capsid as described herein comprises a targeting ligand that binds human TfR, wherein the targeting ligand comprises a heavy chain variable domain, light chain variable domain, CDR1, CDR2, and/or CDR3 amino acid sequence at least 95% identical to, respectively, an amino acid sequence at least 97% identical to an amino acid sequence of a heavy chain variable domain, light chain variable domain, CDR1, CDR2, and/or CDR3 set forth in any one of SEQ ID NOs:1-320 and 365-388. In some embodiments, an AAV capsid as described herein comprises a targeting ligand that binds human TfR, wherein the targeting ligand comprises a heavy chain variable domain, light chain variable domain, CDR1, CDR2, and/or CDR3 amino acid sequence at least 95% identical to, respectively, an amino acid sequence at least 98% identical to an amino acid sequence of a heavy chain variable domain, light chain variable domain, CDR1, CDR2, and/or CDR3 set forth in any one of SEQ ID NOs:1-320 and 365-388. In some embodiments, an AAV capsid as described herein comprises a targeting ligand that binds human TfR, wherein the targeting ligand comprises a heavy chain variable domain, light chain variable domain, CDR1, CDR2, and/or CDR3 amino acid sequence at least 95% identical to, respectively an amino acid sequence at least 99% identical to an amino acid sequence of a heavy chain variable domain, light chain variable domain, CDR1, CDR2, and/or CDR3 set forth in any one of SEQ ID NOs:1-320 and 365-388.

TABLE 1
SEQ ID NOs of Domains in Antibodies, Antigen-binding Fragments (e.g., Fabs) or scFv
Molecules that may be used to retarget AAV to human TfR. SEQ ID NOs in parentheses are nucleic
acids encoding the domain listed.

| Anti-hTfR | | | | | | | | |
|---|---|---|---|---|---|---|---|---|
| ID | HCVR | HCDR1 | HCDR2 | HCDR3 | LCVR | LCDR1 | LCDR2 | LCDR3 |
| H1H31874B | (1) | 3 | 4 | 5 | (6) | 8 | 9 | 10 |
| | 2 or | | | | 7 | | | |
| | 365 | | | | | | | |
| H1H31863B | (11) | 13 | 14 | 15 | (16) | 18 | 19 | 20 |
| | 12 or | | | | 17 | | | |
| | 366 | | | | | | | |
| PN69348 | (21) | 23 | 24 | 25 | (26) | 28 | 29 | 30 |
| | 22 or | | | | 27 | | | |
| | 367 | | | | | | | |
| PN69340 | (31) | 33 | 34 | 35 | (36) | 38 | 39 | 40 |
| | 32 | | | | 37 or | | | |
| | | | | | 368 | | | |
| PN69331 | (41) | 43 | 44 | 45 | (46) | 48 | 49 | 50 |
| | 42 | | | | 47 or | | | |
| | | | | | 369 | | | |
| PN69332 | (51) | 53 | 54 | 55 | (56) | 58 | 59 | 60 |
| | 52 or | | | | 57 or | | | |
| | 370 | | | | 371 | | | |
| PN69326 | (61) | 63 | 64 | 65 | (66) | 68 | 69 | 70 |
| | 62 or | | | | 67 or | | | |
| | 372 | | | | 373 | | | |
| PN69329 | (71) | 73 | 74 | 75 | (76) | 78 | 79 | 80 |
| | 72 or | | | | 77 or | | | |
| | 374 | | | | 375 | | | |
| PN69323 | (81) | 83 | 84 | 85 | (86) | 88 | 89 | 90 |
| | 82 | | | | 87 | | | |
| PN69305 | (91) | 93 | 94 | 95 | (96) | 98 | 99 | 100 |
| | 92 or | | | | 97 | | | |
| | 376 | | | | | | | |
| PN69307 | (101) | 103 | 104 | 105 | (106) | 108 | 109 | 110 |
| | 102 | | | | 107 | | | |
| H1H12795B | (111) | 113 | 114 | 115 | (116) | 118 | 119 | 120 |
| | 112 or | | | | 117 or | | | |
| | 377 | | | | 378 | | | |
| H1H12798B | (121) | 123 | 124 | 125 | (126) | 128 | 129 | 130 |
| | 122 | | | | 127 | | | |
| H1H12799B | (131) | 133 | 134 | 135 | (136) | 138 | 139 | 140 |
| | 132 | | | | 137 | | | |
| H1H12801B | (141) | 143 | 144 | 145 | (146) | 148 | 149 | 150 |
| | 142 or | | | | 147 or | | | |
| | 379 | | | | 380 | | | |
| H1H12802B | (151) | 153 | 154 | 155 | (156) | 158 | 159 | 160 |
| | 152 | | | | 157 | | | |
| H1H12808B | (161) | 163 | 164 | 165 | (166) | 168 | 169 | 170 |
| | 162 or | | | | 167 | | | |
| | 381 | | | | | | | |
| H1H12812B | (171) | 173 | 174 | 175 | (176) | 178 | 179 | 180 |
| | 172 | | | | 177 | | | |
| H1H12816B | (181) | 183 | 184 | 185 | (186) | 188 | 189 | 190 |
| | 182 or | | | | 187 or | | | |
| | 382 | | | | 383 | | | |
| H1H12833B | (191) | 193 | 194 | 195 | (196) | 198 | 199 | 200 |
| | 192 or | | | | 197 | | | |
| | 384 | | | | | | | |
| H1H12834B | (201) | 203 | 204 | 205 | (206) | 208 | 209 | 210 |
| | 202 or | | | | 207 | | | |
| | 385 | | | | | | | |
| H1H12835B | (211) | 213 | 214 | 215 | (216) | 218 | 219 | 220 |
| | 212 | | | | 217 | | | |
| H1H12847B | (221) | 223 | 224 | 225 | (226) | 228 | 229 | 230 |
| | 222 | | | | 227 | | | |
| H1H12848B | (231) | 233 | 234 | 235 | (236) | 238 | 239 | 240 |
| | 232 | | | | 237 | | | |
| H1H12843B | (241) | 243 | 244 | 245 | (246) | 248 | 249 | 250 |
| | 242 | | | | 247 | | | |
| H1H12844B | (251) | 253 | 254 | 255 | (256) | 258 | 259 | 260 |
| | 252 or | | | | 257 | | | |
| | 386 | | | | | | | |
| H1H12845B | (261) | 263 | 264 | 265 | (266) | 268 | 269 | 270 |
| | 262 | | | | 267 | | | |
| H1H12839B | (271) | 273 | 274 | 275 | (276) | 278 | 279 | 280 |
| | 272 | | | | 277 | | | |
| H1H12841B | (281) | 283 | 284 | 285 | (286) | 288 | 289 | 290 |
| | 282 | | | | 287 | | | |
| H1H12850B | (291) | 293 | 294 | 295 | (296) | 298 | 299 | 300 |
| | 292 | | | | 297 | | | |
| PN69261 | (301) | 303 | 304 | 305 | (306) | 308 | 309 | 310 |
| | 302 or | | | | 307 | | | |
| | 387 | | | | | | | |
| PN69263 | (311) | 313 | 314 | 315 | (316) | 318 | 319 | 320 |
| | 312 | | | | 317 or | | | |
| | | | | | 388 | | | |

Non-limiting examples of targeting ligand formats that bind TfR, in addition to bivalent monoclonal antibody (mAb) formats, include: (i) Fab fragments (Fab); (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “targeting ligand,” as used herein. In non-limiting embodiments, an anti-TfR targeting ligand that binds TfR useful for retargeting viral capsids as described herein comprise comprises an scFv. As a non-limiting example, an scFv sequences in V_{L}-(Gly₄Ser)₃-V_{H} format useful for retargeting viral capsids as described herein may comprise an amino acid sequence that is 90%, 95%, 97%, 98%, 99% or 100% identical to any one of the amino acid sequences set forth in SEQ ID NOs:333-364.

In some embodiments, an scFv useful for retargeting viral capsids as described herein may comprise an amino acid sequence that is 90%, 95%, 97%, 98%, 99% or 100% to any one of the amino acid sequences set forth in SEQ ID NOs:333-364, but in VH-(Gly4Ser)3-VL format.

In some cases, the anti-TfR antigen-binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-TfR antibodies and antigen-binding fragments described herein may be modified after translation, e.g., glycosylated.

For example, antibodies and antigen-binding fragments described herein may be glycosylated (e.g., N-glycosylated and/or O-glycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN₂₉₇S or YN₂₉₇S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the anti-TfR antigen-binding protein antibody, i.e., an antibody that does not comprise a glycosylation sequence that might interfere with a transglutamination reaction, for instance an antibody that does not have a saccharide group at N180 and/or N297 on one or more heavy chains. In particular embodiments, an antibody heavy chain has an N180 mutation. In other words, the antibody is mutated to no longer have an asparagine residue at position 180 according to the EU numbering system as disclosed by Kabat et al. In particular embodiments, an antibody heavy chain has an N180Q mutation. In particular embodiments, an antibody heavy chain has an N297 mutation. In particular embodiments, an antibody heavy chain has an N297Q or an N297D mutation. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N180. In some embodiments, deglycosylation is performed at residue N297. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, an antibody disclosed herein comprises Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-TfR antibodies and antigen-binding fragments are provided comprising an Fc domain comprising one or more mutations which enhance or diminish antibody binding to the FcRn receptor, e.g., at acidic pH as compared to neutral pH. For example, the present disclosure includes anti-TfR antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H, F or Y);
   and/or a modification at position:
250 and/or 428;
   and/or a modification at position:
307 or 308 (e.g., 308F, V308F), and/or 434.

In an embodiment, the modification comprises:
a 428L (e.g., M428L) and 434S (e.g., N434S) modification;
a 428L, 259I (e.g., V259I), and 308F (e.g., V308F) modification;
a 433K (e.g., H433K) and a 434 (e.g., 434Y) modification;
a 252, 254, and 256 (e.g, 252Y, 254T, and 256E) modification;
a 250Q and 428L modification (e.g., T250Q and M428L); and/or
a 307 and/or 308 modification (e.g., 308F or 308P).

For example, the present disclosure includes anti-TfR antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
257I and 311I (e.g., P257I and Q311I);
257I and 434H (e.g., P257I and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment, the heavy chain constant domain is gamma4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present disclosure.

The anti-TfR antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fe exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fe portion. In certain embodiments, a “modified Fc domain having reduced effector function” is an Fc domain with reduced or attenuated binding to an Fc receptor (e.g., FcγR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fe hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fe hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fe regions set forth therein.

The present disclosure also includes antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., WO2014/022540).

Other modified Fe domains and Fe modifications that can be used in the context of the present disclosure include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

In some embodiments, the anti-TfR antibodies and antigen-binding fragments described herein comprise an Fc domain comprising one or more mutations in the CH2 and/or CH3 regions that generate a separate TfR binding site.

In an embodiment, the CH2 region comprises one or more amino acid mutations, or a combination thereof, selected from the following: a) position 47 is Glu, Gly, Gln, Ser, Ala, Asn, Tyr, or Trp; position 49 is Ile, Val, Asp, Glu, Thr, Ala, or Tyr; position 56 is Asp, Pro, Met, Leu, Ala, Asn, or Phe; position 58 is Arg, Ser, Ala, or Gly; position 59 is Tyr, Trp, Arg, or Val; position 60 is Glu; position 61 is Trp or Tyr; position 62 is Gln, Tyr, His, Ile, Phe, Val, or Asp; and position 63 is Leu, Trp, Arg, Asn, Tyr, or Val; b) position 39 is Pro, Phe, Ala, Met, or Asp; position 40 is Gln, Pro, Arg, Lys, Ala, Ile, Leu, Glu, Asp, or Tyr; position 41 is Thr, Ser, Gly, Met, Val, Phe, Trp, or Leu; position 42 is Pro, Val, Ala, Thr, or Asp; position 43 is Pro, Val, or Phe; position 44 is Trp, Gln, Thr, or Glu; position 68 is Glu, Val, Thr, Leu, or Trp; position 70 is Tyr, His, Val, or Asp; position 71 is Thr, His, Gln, Arg, Asn, or Val; and position 72 is Tyr, Asn, Asp, Ser, or Pro; c) position 41 is Val or Asp; position 42 is Pro, Met, or Asp; position 43 is Pro or Trp; position 44 is Arg, Trp, Glu, or Thr; position 45 is Met, Tyr, or Trp; position 65 is Leu or Trp; position 66 is Thr, Val, Ile, or Lys; position 67 is Ser, Lys, Ala, or Leu; position 69 is His, Leu, or Pro; and position 73 is Val or Trp; or d) position 45 is Trp, Val, Ile, or Ala; position 47 is Trp or Gly; position 49 is Tyr, Arg, or Glu; position 95 is Ser, Arg, or Gln; position 97 is Val, Ser, or Phe; position 99 is Ile, Ser, or Trp; position 102 is Trp, Thr, Ser, Arg, or Asp; position 103 is Trp; and position 104 is Ser, Lys, Arg, or Val; wherein the substitutions and the positions are determined with reference to amino acids 4-113 of:

(SEQ ID NO: 446)
PCPAPELLGGPSVFLFPPKPKDTLMISRTPEVTCVVVDVSHEDPEV
KFNWYVDGVEVHNAKTKPREEQYNSTYRVVSVLTVLHQDWLNGKE
YKCKVSNKALPAPIEKTISKAKGQPREPQVYTLPPSRDELTKNQV
SLTCLVKGFYPSDIAVEWESNGQPENNYKTTPPVLDSDGSFFLYS
KLTVDKSRWQQGNVFSCSVMHEALHNHYTQKSLSLSPGK.

In an embodiment, the CH3 region comprises one or more amino acid mutations, or a combination thereof, selected from the following: position 153 is Trp, Leu, or Glu; position 157 is Tyr or Phe; position 159 is Thr; position 160 is Glu; position 161 is Trp; position 162 is Ser, Ala, Val, or Asn; position 163 is Ser or Asn; position 186 is Thr or Ser; position 188 is Glu or Ser; position 189 is Glu; and position 194 is Phe; or b) position 118 is Phe or Ile; position 119 is Asp, Glu, Gly, Ala, or Lys; position 120 is Tyr, Met, Leu, Ile, or Asp; position 122 is Thr or Ala; position 210 is Gly; position 211 is Phe; position 212 is His, Tyr, Ser, or Phe; and position 213 is Asp; wherein the substitutions and the positions are determined with reference to amino acids 114-220 of SEQ ID NO: 446.

In some embodiments, the CH3 region comprises one or more mutations, or a combination thereof, selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe, according to EU numbering. In an embodiment, the CH3 region comprises one or more amino acid mutations, or a combination thereof, selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the CH3 region comprises one or more mutations, or a combination thereof, selected from the following: a) Phe at position 382, Tyr at position 383, Asp at position 384, Asp at position 385, Ser at position 386, Lys at position 387, Leu at position 388, Thr at position 389, Pro at position 419, Arg at position 420, Gly at position 421, Leu at position 422, Ala at position 424, Glu at position 426, Tyr at position 438, Leu at position 440, Gly at position 442, and Glu at position 443; b) Phe at position 382, Tyr at position 383, Gly at position 384, N at position 385, Ala at position 386, Lys at position 387, Thr at position 389, Leu at position 422, Ala at position 424, Glu at position 426, Tyr at position 438, Leu at position 440; c) Phe at position 382, Tyr at position 383, Glu at position 384, Ala at position 385, Lys at position 387, Leu at position 388, Leu at position 422, Ala at position 424, Glu at position 426, Tyr at position 438, Leu at position 440; d) Phe at position 382, Glu at position 384, Ser at position 386, Lys at position 387, Thr at position 389, Leu at position 422, Ala at position 424, Glu at position 426, Tyr at position 438, Leu at position 440; e) Phe at position 382, Gly at position 384, Ala at position 385, Lys at position 387, Ser at position 389, Leu at position 422, Ala at position 424, Glu at position 426, Tyr at position 438, Leu at position 440; f) Phe at position 382, Gly at position 384, Ala at position 385, Lys at position 387, Leu at position 388, Thr at position 389, Leu at position 422, Ala at position 424, Glu at position 426, Tyr at position 438, Leu at position 440; wherein the positions are determined according to EU numbering.

Additional mutations in CH2 and/or CH3 regions that can introduce non-native TfR binding sites into the antigen-binding proteins descried herein include those described in US Patent Application Publication Nos. 2020/0223935, 2020/0369746, 2021/0130485, 2022/0017634; and PCT Application Publications Nos. WO2023/279099, WO2023/114499 and WO2023/114510, which are incorporated herein by reference in their entireties.

### Methods of Use and Making

A further embodiment of the modified viral capsid proteins described herein is their use for delivering a nucleotide of interest, e.g., a reporter gene or a therapeutic gene, to a target cell. TfR is widely expressed. Table 2 provides a non-limiting list of tissues and associated cells that may express TfR, and thus, may be targeted by a modified viral capsid protein as described herein for insertion of a nucleotide of interest, e.g., a reporter gene or a therapeutic gene.

TABLE 2

| Target tissue | Cell types |
|---|---|
| Brain/Spinal | endothelial cells |
| cord/CNS/BBB | neurons (all types) |
| | oligodendrocytes (and precursors) |
| | pericytes |
| | meninges/leptomeningeal cells |
| | arachnoid barrier cells |
| | peripheral glia |
| | astrocytes |
| | Glia (all types) |
| | schwann cells |
| | ependymal cells |
| | Neural stem cells |
| | Glioblast cells |
| | microglia |
| Eye | rod photoreceptor cells |
| | muller glia cells |
| | bipolar cells |
| | cone photoreceptor cells |
| | endothelial cells |
| | cornea |
| | sclera |
| | optic nerve |
| | pupillary sphincter |
| Skeletal Muscle | skeletal myocytes |
| | fibroblasts |
| | endothelial cells |
| | macrophages |
| | satellite cells |
| Adipose tissue | Adipocytes |
| | fibroblasts |
| | T-cells |
| | Macrophages |
| | B-cells |
| | Dendritic cells |
| Blood/Bone | T-cells |
| marrow | B-cells |
| | Macrophages |
| | erythroid cells |
| | plasmid cells |
| | dendritic cells |
| Breast | glandular cells |
| | T-cells |
| | fibroblasts |
| | macrophages |
| | endothelial cells |
| | myoepithelial cells |
| | Adipocytes |
| Lung/Bronchus | Basal respiratory cells |
| | respiratory cilliated cells |
| | club cells |
| | smooth muscle cells |
| | ionocytes |
| | macrophages |
| | alveolar cells (type 1 & 2) |
| | T-cells |
| | enothelial cells |
| Colon | distal enterocytes |
| | intestinal goblet cells |
| | undifferentiated cells |
| | T-cells |
| | paneth cells |
| | B-cells |
| | enteroednocrine cells |
| Uterus | glandular and luminal cells |
| | endometrial stromal cells |
| | endothelial cells |
| | smooth muscle cells |
| | T-cells |
| | macrophages |
| Esophagus | fibroblasts |
| | squamous epithelial cells |
| | endothelial cells |
| | smooth muscle cells |
| | macrophages |
| | plasma cells |
| | T-cells |
| Heart | cardiomyocytes |
| | endothelial cells |
| | fibroblasts |
| | macrophages |
| | T-cells |
| | B-cells |
| | Dendritic cells |
| Kidney | proximal tubular cells |
| | T-cells |
| | macrophages |
| | collecting duct cells |
| | B-cells |
| | glomeruli |
| | fibroblasts |
| Liver | hepatocytes |
| | B-cells |
| | erythroid cells |
| Lymph node | B-cells |
| | T-cells |
| Ovary | granulosa cells |
| | fibroblasts |
| | smooth muscle cells |
| | macrophages |
| | T-cells |
| | theca cells |
| | fibroblasts |
| Pancreas | ductal cells |
| | pancreatic endocrine cells |
| | smooth muscle cells |
| | endothelial cells |
| | macrophages |
| | exocrine glandular cells |
| | monocytes |
| Placenta | cytotrophoblasts |
| | extravillous trophoblasts |
| | fibroblasts |
| | hofbauer cells |
| | endothelial cells |
| Prostate | basal prostatic cells |
| | prostatic glandular cells |
| | urothelial cells |
| | endothelial cells |
| | fibroblasts |
| | smooth muscle cells |
| | macrophages |
| | T-cells |
| Rectum | undifferentiated cells |
| | intestinal goblet cells |
| | paneth cells |
| | distal enterocytes |
| | enteroednocrine cells |
| Skin | langerhans cells |
| | fibroblasts |
| | endothelial cells |
| | basal keratinocytes |
| | suprabasal keratinocytes |
| | T-cells |
| | smooth muscle cells |
| | melanocytes |
| PBMC | monocytes |
| | T-cells |
| | NK-cells |
| | dendritic cells |
| Small intestine | proximal enterocytes |
| | undifferentiated cells |
| | intestinal goblet cells |
| | paneth cells |
| Spleen | B-cells |
| | T-cells |
| | plasma cells |
| | macrophages |
| Stomach | B-cells |
| | T-cells |
| | gastric mucus-secreting cells |
| | plasma cells |
| | fibroblasts |
| | macrophages |
| Testis | leydig cells |
| | late spermatids |
| | spermatogonia |
| | early spermatids |
| | macrophages |
| | spermatocytes |
| | peritubular cells |
| | sertoli cells |
| | endothelial cells |
| Peripheral nervous | motor neurons |
| system | sensory neurons |
| | schwann cells |
| | Peripheral glia (all types) |
| | Sensory neurons |
| | Satellite glial cells |
| | Enteric glial cells |
| | Olfactory ensheathing cells |
| | dorsal root ganglion cells (all types) |
| Bone/cartilage/joint | chondrocytes |
| | chondroblasts |
| | mesenchymal cells |
| | osteoblasts |
| | osteoclasts |

Generally, a nucleotide of interest may be a transfer plasmid, which may generally comprise 5′ and 3′ inverted terminal repeat (ITR) sequences flanking the reporter gene(s) or therapeutic gene(s) (which may be under the control of a viral or non-viral promoter, when encompassed within an AAV particle). In one embodiment, a nucleotide of interest is a transfer plasmid comprising from 5′ to 3′: a 5′ ITR, a promoter, a gene (e.g., a reporter and/or therapeutic gene) and a 3′ITR.

Non-limiting examples of useful promoters include, e.g., cytomegalovirus (CMV)-promoter, chicken beta actin (CBA) promoter and a hybrid thereof (CBh) the spleen focus forming virus (SFFV)-promoter, the elongation factor 1 alpha (EF1a)-promoter (the 1.2 kb EF1a-promoter or the 0.2 kb EF1a-promoter), the chimeric EF 1 a/IF4-promoter, the polyubiquitin C promoter (UbC), and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, the CMV enhancer can be used in combination with the chicken β-actin promoter, e.g., as a hybrid (CAG). Alternatively, the promoter may be a tissue-specific promoter, i.e., it is active in specific tissue(s) and/or organ(s). A tissue-specific promoter comprises one or more tissue-specific promoter and/or enhancer elements, and optionally one or more constitutive promoter and/or enhancer elements as described in US 2022/0204991, which is incorporated by reference herein in its entirety. A skilled artisan would appreciate that tissue-specific promoter and/or enhancer elements can be isolated from genes specifically expressed in the tissue by methods well known in the art.

A variety of reporter genes (or detectable moieties) can be encapsidated in a multimeric structure comprising the modified viral capsid proteins described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting particles that employ the use of a reporter gene that encodes green fluorescent protein, however, persons of skill upon reading this disclosure will understand that the viral capsids described herein can be generated in the absence of a reporter gene or with any reporter gene known in the art.

A variety of therapeutic genes can also be encapsidated in a multimeric structure comprising the modified viral capsid proteins described herein, e.g., as part of a transfer particle. Non-limiting examples of a therapeutic gene include those that encode a toxin (e.g., a suicide gene), a therapeutic antibody or fragment thereof, a CRISPR/Cas system or portion(s) thereof, antisense oligonucleotides, antisense RNA, siRNA, shRNA, etc. Tables 3 and 4 provide a non-limiting list of diseases and the genes, which may be a nucleotide of interest and/or for which the reduction of which may be therapeutic, that may be suitable for treatment using the viral particles described herein.

TABLE 3

| Disease | Target |
|---|---|
| Spinal muscular atrophy SMA | SMN1 |
| Lysosomal storage diseases with central | Genes encoding the |
| nervous system manifestations | deficient enzyme |
| (neuropathic); Fabry disease, Gaucher | |
| disease, Pompe disease and many more | |
| Parkinson's/synucleinopathies | Alpha-synuclein SNCA |
| Parkinson's | Lrrk2 |
| Parkinson's, Gaucher disease | GBA |
| FTD | Progranulin |
| Alzheimer's/tauopathies | 3R/4R Tau |
| | 4R-specific Tau |
| Alzheimer's Disease | APP/Abeta |
| | Trem2 |
| | ApoE |
| ALS/FTD/TDP-43 proteinopathies | SOD1 |
| | C9orf72 |
| | TDP43 |
| | SUPT4H1 |
| | PUMA |
| | ELOVL1 |
| | RPS25 |
| | PSMB5 |
| Huntington's disease | HTT |
| Transthyretin amyloidosis | TTR |
| Friedreich's Ataxia | FXN |
| Pain | Nav1.7 |
| | Nav1.9 |
| | TrpAl |
| | TRPM8 |
| | TrkA |
| | FXYD2 |
| | AAK1 |
| | ASIC3 |
| | ASIC1 |
| | NGF |

TABLE 4

| Tissue/ | | |
|---|---|---|
| Organelle | Gene Name | OMIM disease name |
| Lysosomal targets | ABCA1 | Tangier disease |
| | | Intellectual developmental disorder with poor |
| | ABCA2 | growth and with or without seizures or ataxia |
| | | Hypermethioninemia due to adenosine kinase |
| | ADK | deficiency |
| | AGA | Aspartylglycosaminuria |
| | ALDOB | Fructose intolerance, hereditary |
| | AP1S1 | MEDNIK syndrome |
| | AP4M1 | Spastic paraplegia 50, autosomal recessive |
| | APOE | Sea-blue histiocyte disease |
| | APRT | Adenine phosphoribosyltransferase deficiency |
| | | Maroteaux-Lamy syndrome |
| | ARSB | (mucopolysaccharidosis type VI |
| | ARSK | Mucopolysaccharidosis, type X |
| | ATG7 | Spinocerebellar ataxia, autosomal recessive 31 |
| | ATP6VIA | Cutis laxa, autosomal recessive, type IID |
| | ASAH1 | Farber disease |
| | BLOC1S6 | Hermansky-Pudlak syndrome 9 |
| | CLN6 | Ceroid lipofuscinosis, neuronal, 6A |
| | CLN6 | Ceroid lipofuscinosis, neuronal, 6B (Kufs type) |
| | CLN8 | Ceroid lipofuscinosis, neuronal, 8 |
| | | Ceroid lipofuscinosis, neuronal, 8, Northern |
| | CLN8 | epilepsy variant |
| | CTSA | Galactosialidosis |
| | CTNS | cystinosis |
| | | Haim-Munk syndrome, Papillon Lefevre |
| | CTSC | Syndrome |
| | CTSD | Ceroid lipofuscinosis, neuronal, 10 |
| | CTSK | Pycnodysostosis |
| | CUBN | Imerslund-Grasbeck syndrome 1 |
| | CUBN | Proteinuria, chronic benign] |
| | CXCR2 | WHIM syndrome 2 |
| | CYB561 | Orthostatic hypotension 2 |
| | DPYD | 5-fluorouracil toxicity |
| | DPYD | Dihydropyrimidine dehydrogenase deficiency |
| | DRAM2 | Cone-rod dystrophy 21 |
| | EPG5 | Vici syndrome |
| | | Arthrogryposis multiplex congenita 2, |
| | ERGIC1 | neurogenic type |
| | FUCA1 | Fucosidosis |
| | FYCO1 | Cataract 18, autosomal recessive |
| | GAA | Pompe disease |
| | GALNS | Mucopolysaccharidosis IV |
| | GBA | Gaucher disease |
| | GLA | Fabry disease |
| | GNPTAB | Mucolipidosis II alpha/beta |
| | GNPTAB | Mucolipidosis III alpha/beta |
| | GNPTG | Mucolipidosis III gamma |
| | GUSB | Mucopolysaccharidosis Type VII |
| | HEXA | Tay Sachs Disease |
| | HEXB | Sandhoff disease, infantile, juvenile, and adult |
| | HGSNAT | forms |
| | | Mucopolysaccharidosis type IIIC (Sanfilippo |
| | | C) |
| | HGSNAT | Retinitis pigmentosa 73 |
| | HPS6 | Hermansky-Pudlak syndrome 6 |
| | IDUA | Mucopolysaccharidosis I |
| | IDS | Mucopolysaccharidosis II |
| | KLC2 | Spastic paraplegia, optic atrophy, and |
| | | neuropathy |
| | LAL | Lysosomal acid lipase defciency |
| | LAMP2 | Danon disease |
| | LAT | Immunodeficiency 52 |
| | LHCGR | Leydig cell hypoplasia with hypergonadotropic |
| | | hypogonadism |
| | LHCGR | Leydig cell hypoplasia with |
| | | pseudohermaphroditism |
| | LHCGR | Luteinizing hormone resistance, female |
| | LRBA | Immunodeficiency, common variable, 8, with |
| | | autoimmunity |
| | LRP1 | Keratosis pilaris atrophicans |
| | LYST | Chediak-Higashi syndrome |
| | MAN2B1 | Alpha-Mannosidosis |
| | MBTPS1 | Spondyloepiphyseal Dysplasia, Kondo-Fu Type |
| | MCOLN1 | Mucolipidosis IV |
| | MFSD8 | Ceroid lipofuscinosis, neuronal, 7 |
| | | Macular dystrophy with central cone |
| | MFSD8 | involvement |
| | | Megalencephalic leukoencephalopathy with |
| | MLC1 | subcortical cysts |
| | MPO | Myeloperoxidase deficiency |
| | MYO7A | Deafness, autosomal recessive 2 |
| | MYO7A | Usher syndrome, type 1B |
| | NAGA | Kanzaki disease |
| | NAGA | Schindler disease, type I |
| | NAGA | Schindler disease, type III |
| | NPC1 | Niemann-Pick disease, type C1 |
| | NPC1 | Niemann-Pick disease, type D |
| | NPC2 | Niemann-pick disease, type C2 |
| | NT5C2 | Spastic paraplegia 45, autosomal recessive |
| | NEU1 | Sialidosis |
| | PINK1 | Parkinson disease 6, early onset |
| | PLEKHM1 | Osteopetrosis, autosomal recessive 6 |
| | PRF1 | Hemophagocytic lymphohistiocytosis, familial, |
| | | 2 |
| | SCARB2 | Epilepsy, progressive myoclonic 4, with or |
| | | without renal failure |
| | SGSH | Mucopolysaccharidosis type IIIA (Sanfilippo |
| | | A) |
| | SHMT2 | Neurodevelopmental disorder with |
| | | cardiomyopathy, spasticity, and brain |
| | | abnormalities |
| | SLC29A3 | Histiocytosis-lymphadenopathy plus syndrome |
| | SMPD1 | Niemann-Pick disease, type A/B, acid |
| | | sphingomyelinase deficiency |
| | SLC39A8 | Congenital disorder of glycosylation, type IIn |
| | SNX14 | Spinocerebellar ataxia, autosomal recessive 20 |
| | SPG11 | Amyotrophic lateral sclerosis 5, juvenile |
| | SPG11 | Charcot-Marie-Tooth disease, axonal, type 2X |
| | SPG11 | Spastic paraplegia 11, autosomal recessive |
| | TBC1D20 | Warburg micro syndrome 4 |
| | VPS11 | Dystonia 32 |
| | VPS11 | Leukodystrophy, hypomyelinating, 12 |
| | VPS13A | Choreoacanthocytosis |
| | VPS33B | Arthrogryposis, renal dysfunction, and |
| | | cholestasis 1 |
| | VPS51 | Pontocerebellar hypoplasia, type 13 |
| | VPS53 | Pontocerebellar hypoplasia, type 2E |
| | WDR45B | Neurodevelopmental disorder with spastic |
| | | quadriplegia and brain abnormalities with or |
| | | without seizures |
| | WDR81 | Cerebellar ataxia, mental retardation, and |
| | | dysequilibrium syndrome 2 |
| | WDR81 | Hydrocephalus, congenital, 3, with brain |
| | | anomalies |
| | XDH | Xanthinuria, type I |
| | ZFYVE26 | Spastic paraplegia 15, autosomal recessive |
| | ABCC9 | Intellectual disability and myopathy syndrome |
| Heart | BVES | Muscular dystrophy, limb-girdle, autosomal |
| | | recessive 25 |
| | CACNA1B | Neurodevelopmental disorder with seizures and |
| | | nonepileptic hyperkinetic movements |
| | CACNA2D2 | Cerebellar atrophy with seizures and variable |
| | | developmental delay |
| | CASQ2 | Ventricular tachycardia, catecholaminergic |
| | | polymorphic, 2 |
| | CAV1 | Lipodystrophy, congenital generalized, type 3 |
| | DSC2 | Arrhythmogenic right ventricular dysplasia 11 |
| | DSC2 | Arrhythmogenic right ventricular dysplasia 11 |
| | | with mild palmoplantar keratoderma and |
| | | woolly hair |
| | DSP | Cardiomyopathy, dilated, with woolly hair and |
| | | keratoderma |
| | DSP | Epidermolysis bullosa, lethal acantholytic |
| | DSP | Skin fragility-woolly hair syndrome |
| | GATA5 | Congenital heart defects, multiple types, 5 |
| | GPX1 | Hemolytic anemia due to glutathione |
| | | peroxidase deficiency |
| | JUP | Naxos disease |
| | KCNE1 | Jervell and Lange-Nielsen syndrome 2 |
| | MYL2 | Myopathy, myofibrillar, 12, infantile-onset, |
| | | with cardiomyopathy |
| | MYL3 | Cardiomyopathy, hypertrophic, 8 |
| | NOS1AP | Nephrotic syndrome, type 22 |
| | SCN1B | Developmental and epileptic encephalopathy 52 |
| | SLC1A1 | Dicarboxylic aminoaciduria |
| | SLC9A1 | Lichtenstein-Knorr syndrome |
| | | Hypogonadotropic hypogonadism 11 with or |
| | TACR3 | without anosmia |
| | TH | Segawa syndrome, recessive |
| Central nervous system | ABCA2 | Intellectual developmental disorder with poor |
| | | growth and with or without seizures or ataxia |
| | ACAN | Spondyloepimetaphyseal dysplasia, aggrecan |
| | | type |
| | ADARB1 | Neurodevelopmental disorder with hypotonia, |
| | | microcephaly, and seizures |
| | ANKLE2 | Microcephaly 16, primary, autosomal recessive |
| | ATG7 | Spinocerebellar ataxia, autosomal recessive 31 |
| | BMPR1B | Acromesomelic dysplasia 3 |
| | CDH11 | Elsahy-Waters syndrome |
| | CLN8 | Ceroid lipofuscinosis, neuronal, 8 |
| | CLN8 | Ceroid lipofuscinosis, neuronal, 8, Northern |
| | | epilepsy variant |
| | CNTNAP2 | Pitt-Hopkins like syndrome 1 |
| | DCC | Gaze palsy, familial horizontal, with |
| | | progressive scoliosis, 2 |
| | DYNC2H1 | Short-rib thoracic dysplasia 3 with or without |
| | | polydactyly |
| | EPHB2 | Bleeding disorder, platelet-type, 22 |
| | HERC1 | Macrocephaly, dysmorphic facies, and |
| | | psychomotor retardation |
| | IGHMBP2 | Charcot-Marie-Tooth disease, axonal, type 2S |
| | IGHMBP2 | Neuronopathy, distal hereditary motor, type VI |
| | KCNJ10 | SESAME syndrome |
| | KIFBP | Goldberg-Shprintzen megacolon syndrome |
| | LEP | Obesity, morbid, due to leptin deficiency |
| | MAG | Spastic paraplegia 75, autosomal recessive |
| | NHLH2 | Hypogonadotropic hypogonadism 27 without |
| | | anosmia |
| | NIN | Seckel syndrome 7 |
| | NRXN1 | Pitt-Hopkins-like syndrome 2 |
| | OGDH | Oxoglutarate dehydrogenase deficiency |
| | PAX7 | Myopathy, congenital, progressive, with |
| | | scoliosis |
| | PRDM8 | Epilepsy, progressive myoclonic, 10 |
| | RELN | Lissencephaly 2 (Norman-Roberts type) |
| | SECISBP2 | Thyroid hormone metabolism, abnormal |
| | SECISBP2 | Thyroid hormone metabolism, abnormal, 1 |
| | SLC25A46 | Neuropathy, hereditary motor and sensory, type |
| | | VIB |
| | SLC25A46 | Pontocerebellar hypoplasia, type 1E |
| | SPG11 | Amyotrophic lateral sclerosis 5, juvenile |
| | SPG11 | Charcot-Marie-Tooth disease, axonal, type 2X |
| | SPG11 | Spastic paraplegia 11, autosomal recessive |
| | SPINK5 | Netherton syndrome |
| | TCTN1 | Joubert syndrome 13 |
| | VAX1 | Microphthalmia, syndromic 11 |
| | WNT1 | Osteogenesis imperfecta, type XV |
| | Eye ABCA2 | Intellectual developmental disorder with poor |
| | | growth and with or without seizures or ataxia |
| | ADAMTS18 | Microcornea, myopic chorioretinal atrophy, and |
| | | telecanthus |
| | ALDH1A3 | Microphthalmia, isolated 8 |
| | ALDOB | Fructose intolerance, hereditary |
| | ALMS1 | Alstrom syndrome |
| | APOE | Sea-blue histiocyte disease |
| | ARSK | Mucopolysaccharidosis, type X |
| | ATP6V1A | Cutis laxa, autosomal recessive, type IID |
| | BBS4 | Bardet-Biedl syndrome 4 |
| | BBS7 | Bardet-Biedl syndrome 7 |
| | BMPR1B | Acromesomelic dysplasia 3 |
| | CABP4 | Cone-rod synaptic disorder, congenital |
| | | nonprogressive |
| | CEP290 | Joubert syndrome 5 |
| | CEP290 | Leber congenital amaurosis 10 |
| | CEP290 | Meckel syndrome 4 |
| | CEP290 | Senior-Loken syndrome 6 |
| | CFD | Complement factor D deficiency |
| | CLN8 | Ceroid lipofuscinosis, neuronal, 8 |
| | CLN8 | Ceroid lipofuscinosis, neuronal, 8, Northern |
| | | epilepsy variant |
| | CNGA3 | Achromatopsia 2 |
| | CRB2 | Focal segmental glomerulosclerosis 9 |
| | CRB2 | Ventriculomegaly with cystic kidney disease |
| | CRX | Leber congenital amaurosis 7 |
| | CRYBB3 | Cataract 22 |
| | CTSA | Galactosialidosis |
| | CTSD | Ceroid lipofuscinosis, neuronal, 10 |
| | CTSK | Pycnodysostosis |
| | CUBN | Imerslund-Grasbeck syndrome 1 |
| | CUBN | Proteinuria, chronic benign] |
| | CXCR2 | WHIM syndrome 2 |
| | DRAM2 | Cone-rod dystrophy 21 |
| | EPG5 | Vici syndrome |
| | EPHB2 | Bleeding disorder, platelet-type, 22 |
| | FOXE3 | Anterior segment dysgenesis 2, multiple |
| | | subtypes |
| | FUCA1 | Fucosidosis |
| | FYCO1 | Cataract 18, autosomal recessive |
| | GRHL2 | Ectodermal dysplasia/short stature syndrome |
| | GRM6 | Night blindness, congenital stationary |
| | | (complete), 1B, autosomal recessive |
| | HESX1 | Growth hormone deficiency with pituitary |
| | | anomalies |
| | HESX1 | Pituitary hormone deficiency, combined, 5 |
| | HESX1 | Septooptic dysplasia |
| | HEXB | Sandhoff disease, infantile, juvenile, and adult |
| | | forms |
| | HGSNAT | Mucopolysaccharidosis type IIIC (Sanfilippo |
| | | C) |
| | HGSNAT | Retinitis pigmentosa 73 |
| | HPS6 | Hermansky-Pudlak syndrome 6 |
| | KCNMA1 | Cerebellar atrophy, developmental delay, and |
| | | seizures |
| | KERA | Cornea plana 2, autosomal recessive |
| | KLC2 | Spastic paraplegia, optic atrophy, and |
| | | neuropathy |
| | LAMA1 | Poretti-Boltshauser syndrome |
| | LAMC3 | Cortical malformations, occipital |
| | LHCGR | Leydig cell hypoplasia with hypergonadotropic |
| | | hypogonadism |
| | LHCGR | Leydig cell hypoplasia with |
| | | pseudohermaphroditism |
| | LHCGR | Luteinizing hormone resistance, female |
| | LRBA | Immunodeficiency, common variable, 8, with |
| | | autoimmunity |
| | MAB21L2 | Microphthalmia/coloboma and skeletal |
| | | dysplasia syndrome |
| | MFN2 | Charcot-Marie-Tooth disease, axonal, type |
| | | 2A2B |
| | MFSD2A | Neurodevelopmental disorder with progressive |
| | | microcephaly, spasticity, and brain |
| | | abnormalities |
| | MFSD8 | Ceroid lipofuscinosis, neuronal, 7 |
| | MFSD8 | Macular dystrophy with central cone |
| | | involvement |
| | MLC1 | Megalencephalic leukoencephalopathy with |
| | | subcortical cysts |
| | MPO | Myeloperoxidase deficiency |
| | NAGA | Kanzaki disease |
| | NAGA | Schindler disease, type I |
| | NAGA | Schindler disease, type III |
| | NPC1 | Niemann-Pick disease, type C1 |
| | NPC1 | Niemann-Pick disease, type D |
| | NPC2 | Niemann-pick disease, type C2 |
| | NPHP1 | Joubert syndrome 4 |
| | NPHP1 | Nephronophthisis 1, juvenile |
| | NPHP1 | Senior-Loken syndrome-1 |
| | PCNT | Microcephalic osteodysplastic primordial |
| | | dwarfism, type II |
| | PDE6A | Retinitis pigmentosa 43 |
| | PDE6B | Retinitis pigmentosa-40 |
| | PITX3 | Cataract 11, multiple types |
| | PITX3 | Cataract 11, syndromic, autosomal recessive |
| | PLEKHM1 | Osteopetrosis, autosomal recessive 6 |
| | PRF1 | Hemophagocytic lymphohistiocytosis, familial, |
| | | 2 |
| | PRSS56 | Microphthalmia, isolated 6 |
| | PXDN | Anterior segment dysgenesis 7, with |
| | | sclerocornea |
| | RAB3GAP1 | Martsolf syndrome 2 |
| | RAB3GAP1 | Warburg micro syndrome 1 |
| | RBP4 | Retinal dystrophy, iris coloboma, and |
| | | comedogenic acne syndrome |
| | RD3 | Leber congenital amaurosis 12 |
| | RPGRIP1L | COACH syndrome 3 |
| | RPGRIP1L | Joubert syndrome 7 |
| | RPGRIP1L | Meckel syndrome 5 |
| | | Intellectual developmental disorder and retinitis |
| | SCAPER | pigmentosa |
| | | Epilepsy, progressive myoclonic 4, with or |
| | SCARB2 | without renal failure |
| | SGSH | Mucopolysaccharidosis type IIIA (Sanfilippo |
| | | A) |
| | SLC1A1 | Dicarboxylic aminoaciduria |
| | SLC29A3 | Histiocytosis-lymphadenopathy plus syndrome |
| | SLC39A8 | Congenital disorder of glycosylation, type IIn |
| | SMG9 | Heart and brain malformation syndrome |
| | SMOC1 | Microphthalmia with limb anomalies |
| | SNX14 | Spinocerebellar ataxia, autosomal recessive 20 |
| | SPNS2 | Deafness, autosomal recessive 115 |
| | TBC1D20 | Warburg micro syndrome 4 |
| | TH | Segawa syndrome, recessive |
| | TRPM1 | Night blindness, congenital stationary |
| | | (complete), 1C, autosomal recessive |
| | TWIST2 | Focal facial dermal dysplasia 3, Setleis type |
| | USH1C | Deafness, autosomal recessive 18A |
| | USH1C | Usher syndrome, type 1C |
| | VAX1 | Microphthalmia, syndromic 11 |
| | VPS11 | Dystonia 32 |
| | VPS11 | Leukodystrophy, hypomyelinating, 12 |
| | VPS13A | Choreoacanthocytosis |
| | VPS33B | Arthrogryposis, renal dysfunction, and |
| | | cholestasis 1 |
| | WDR45B | Neurodevelopmental disorder with spastic |
| | | quadriplegia and brain abnormalities with or |
| | | without seizures |
| | WDR81 | Cerebellar ataxia, mental retardation, and |
| | | dysequilibrium syndrome 2 |
| | WDR81 | Hydrocephalus, congenital, 3, with brain |
| | | anomalies |
| | ZFYVE26 | Spastic paraplegia 15, autosomal recessive |
| Brain | ADCY1 | Deafness, autosomal recessive 44 |
| | ASPM | Microcephaly 5, primary, autosomal recessive |
| | ATG7 | Spinocerebellar ataxia, autosomal recessive 31 |
| | BBS2 | Bardet-Biedl syndrome 2 |
| | BBS2 | Retinitis pigmentosa 74 |
| | BBS4 | Bardet-Biedl syndrome 4 |
| | CNTNAP2 | Pitt-Hopkins like syndrome 1 |
| | CPLANE1 | Joubert syndrome 17 |
| | CPLANE1 | Orofaciodigital syndrome VI |
| | DCT | Oculocutaneous albinism, type VIII |
| | DTNBP1 | Hermansky-Pudlak syndrome 7 |
| | DYNC2H1 | Short-rib thoracic dysplasia 3 with or without |
| | | polydactyly |
| | HERC1 | Macrocephaly, dysmorphic facies, and |
| | | psychomotor retardation |
| | HESX1 | Growth hormone deficiency with pituitary |
| | | anomalies |
| | HESX1 | Pituitary hormone deficiency, combined, 5 |
| | HESX1 | Septooptic dysplasia |
| | MBOAT7 | Intellectual developmental disorder, autosomal |
| | | recessive 57 |
| | MFSD2A | Neurodevelopmental disorder with progressive |
| | | microcephaly, spasticity, and brain |
| | | abnormalities |
| | NHLH2 | Hypogonadotropic hypogonadism 27 without |
| | | anosmia |
| | NRXN1 | Pitt-Hopkins-like syndrome 2 |
| | OGDH | Oxoglutarate dehydrogenase deficiency |
| | PCNT | Microcephalic osteodysplastic primordial |
| | | dwarfism, type II |
| | PDE2A | Intellectual developmental disorder with |
| | | paroxysmal dyskinesia or seizures |
| | PGAP1 | Neurodevelopmental disorder with dysmorphic |
| | | features, spasticity, and brain abnormalities |
| | PLCB1 | Developmental and epileptic encephalopathy 12 |
| | RAB3GAP1 | Martsolf syndrome 2 |
| | RAB3GAP1 | Warburg micro syndrome 1 |
| | RELN | Lissencephaly 2 (Norman-Roberts type) |
| | RPGRIP1L | COACH syndrome 3 |
| | RPGRIP1L | Joubert syndrome 7 |
| | RPGRIP1L | Meckel syndrome 5 |
| | SECISBP2 | Thyroid hormone metabolism, abnormal |
| | SECISBP2 | Thyroid hormone metabolism, abnormal, 1 |
| | SLC25A46 | Neuropathy, hereditary motor and sensory, type |
| | | VIB |
| | SLC25A46 | Pontocerebellar hypoplasia, type 1E |
| | SPTBN2 | Spinocerebellar ataxia, autosomal recessive 14 |
| | STAMBP | Microcephaly-capillary malformation syndrome |
| | TNR | Neurodevelopmental disorder, nonprogressive, |
| | | with spasticity and transient opisthotonus |
| | TRAPPC9 | Intellectual developmental disorder, autosomal |
| | | recessive 13 |
| | WDR62 | Microcephaly 2, primary, autosomal recessive, |
| | | with or without cortical malformations |
| | WNT1 | Osteogenesis imperfecta, type XV |
| | WNT2B | Diarrhea 9 |
| Spinal cord | ADARB1 | Neurodevelopmental disorder with hypotonia, |
| | | microcephaly, and seizures |
| | CLN8 | Ceroid lipofuscinosis, neuronal, 8 |
| | CLN8 | Ceroid lipofuscinosis, neuronal, 8, Northern |
| | | epilepsy variant |
| | DAAM2 | Nephrotic syndrome, type 24 |
| | DCC | Gaze palsy, familial horizontal, with |
| | | progressive scoliosis, 2 |
| | DYNC2H1 | Short-rib thoracic dysplasia 3 with or without |
| | | polydactyly |
| | IGHMBP2 | Charcot-Marie-Tooth disease, axonal, type 2S |
| | IGHMBP2 | Neuronopathy, distal hereditary motor, type VI |
| | PAX7 | Myopathy, congenital, progressive, with |
| | | scoliosis |
| | PHGDH | Neu-Laxova syndrome 1 |
| | PHGDH | Phosphoglycerate dehydrogenase deficiency |
| | RAB23 | Carpenter syndrome |
| | RELN | Lissencephaly 2 (Norman-Roberts type) |
| | TCTN1 | Joubert syndrome 13 |
| | VLDLR | Cerebellar hypoplasia and mental retardation |
| | | with or without quadrupedal locomotion 1 |
| | WNT1 | Osteogenesis imperfecta, type XV |
| Peripheral nervous | ERBB2 | Visceral neuropathy, familial, 2, autosomal |
| system | | recessive |
| | LGI4 | Arthrogryposis multiplex congenita 1, |
| | | neurogenic, with myelin defect |
| | MMP2 | Multicentric osteolysis, nodulosis, and |
| | | arthropathy |
| | NDRG1 | Charcot-Marie-Tooth disease, type 4D |
| | NHLH2 | Hypogonadotropic hypogonadism 27 without |
| | | anosmia |
| | SH3TC2 | Charcot-Marie-Tooth disease, type 4C |
| | SLC25A46 | Neuropathy, hereditary motor and sensory, type |
| | | VIB |
| | SLC25A46 | Pontocerebellar hypoplasia, type 1E |
| | TBCE | Encephalopathy, progressive, with amyotrophy |
| | | and optic atrophy |
| | TBCE | Hypoparathyroidism-retardation-dysmorphism |
| | | syndrome |
| | TBCE | Kenny-Caffey syndrome, type 1 |
| Skeletal muscle | ATP2A1 | Brody myopathy |
| | | Muscular dystrophy, limb-girdle, autosomal |
| | BVES | recessive 25 |
| | CAV1 | Lipodystrophy, congenital generalized, type 3 |
| | | Myasthenic syndrome, congenital, 1B, fast- |
| | CHRNA1 | channel |
| | | Myasthenic syndrome, congenital, 3B, fast- |
| | CHRND | channel |
| | | Myasthenic syndrome, congenital, 3C, |
| | | associated with acetylcholine receptor |
| | CHRND | deficiency |
| | CLN8 | Ceroid lipofuscinosis, neuronal, 8 |
| | | Ceroid lipofuscinosis, neuronal, 8, Northern |
| | CLN8 | epilepsy variant |
| | FLNB | Spondylocarpotarsal synostosis syndrome |
| | | Hemolytic anemia due to glutathione |
| | GPX1 | peroxidase deficiency |
| | ITPR1 | Gillespie syndrome |
| | LMOD3 | Nemaline myopathy 10 |
| | | Myopathy, congenital, progressive, with |
| | PAX7 | scoliosis |
| | SCN4A | Myasthenic syndrome, congenital, 16 |
| | | Dystonia, dopa-responsive, due to sepiapterin |
| | SPR | reductase deficiency |
| | WNT10B | Split-hand/foot malformation 6 |
| Cartilage | ACAN | Spondyloepimetaphyseal dysplasia, aggrecan |
| | | type |
| | BBS2 | Bardet-Biedl syndrome 2 |
| | BBS2 | Retinitis pigmentosa 74 |
| | BMPR1B | Acromesomelic dysplasia 3 |
| | CHST11 | Osteochondrodysplasia, brachydactyly, and |
| | | overlapping malformed digits |
| | CHSY1 | Temtamy preaxial brachydactyly syndrome |
| | COL11A1 | Fibrochondrogenesis 1 |
| | COL11A2 | Deafness, autosomal recessive 53 |
| | COL11A2 | Fibrochondrogenesis 2 |
| | COL11A2 | Otospondylomegaepiphyseal dysplasia, |
| | | autosomal recessive |
| | COL27A1 | Steel syndrome |
| | CTSK | Pycnodysostosis |
| | DDRGK1 | Spondyloepimetaphyseal dysplasia, Shohat type |
| | GDF5 | Acromesomelic dysplasia 2A |
| | GDF5 | Acromesomelic dysplasia 2B |
| | GDF5 | Acromesomelic dysplasia 2C, Hunter- |
| | | Thompson type |
| | GDF5 | Brachydactyly, type A1, C |
| | GDF6 | Leber congenital amaurosis 17 |
| | IFT80 | Short-rib thoracic dysplasia 2 with or without |
| | | polydactyly |
| | LEP | Obesity, morbid, due to leptin deficiency |
| | LNPK | Neurodevelopmental disorder with epilepsy and |
| | | hypoplasia of the corpus callosum |
| | PAX7 | Myopathy, congenital, progressive, with |
| | | scoliosis |
| | PKDCC | Rhizomelic limb shortening with dysmorphic |
| | | features |
| | POC1A | Short stature, onychodysplasia, facial |
| | | dysmorphism, and hypotrichosis |
| | PTH | Hypoparathyroidism, familial isolated 1 |
| | ROR2 | Robinow syndrome, autosomal recessive |
| | SIK3 | Spondyloepimetaphyseal dysplasia, Krakow |
| | | type |
| | SLC39A8 | Congenital disorder of glycosylation, type IIn |
| | SNAI2 | Waardenburg syndrome, type 2D |
| Bone growth plate | COL1A2 | Ehlers-Danlos syndrome, cardiac valvular type |
| | COL27A1 | Steel syndrome |
| | F7 | Factor VII deficiency |
| | IFT80 | Short-rib thoracic dysplasia 2 with or without |
| | | polydactyly |
| | LRP1 | Keratosis pilaris atrophicans |
| | POC1A | Short stature, onychodysplasia, facial |
| | | dysmorphism, and hypotrichosis |
| | SGPL1 | Nephrotic syndrome, type 14 |
| | SNAI2 | Waardenburg syndrome, type 2D |
| Kidney | ABCA4 | Cone-rod dystrophy 3 |
| | ABCA4 | Fundus flavimaculatus |
| | ABCA4 | Retinal dystrophy, early-onset severe |
| | ABCA4 | Retinitis pigmentosa 19 |
| | ABCA4 | Stargardt disease 1 |
| | ADCY6 | Lethal congenital contracture syndrome 8 |
| | ANKS6 | Nephronophthisis 16 |
| | ATP6V0A4 | Distal renal tubular acidosis 3, with or without |
| | | sensorineural hearing loss |
| | ATP6V1B1 | Distal renal tubular acidosis 2 with progressive |
| | | sensorineural hearing loss |
| | BBS2 | Bardet-Biedl syndrome 2 |
| | BBS2 | Retinitis pigmentosa 74 |
| | CABP2 | Deafness, autosomal recessive 93 |
| | CABP4 | Cone-rod synaptic disorder, congenital |
| | | nonprogressive |
| | CEP290 | Joubert syndrome 5 |
| | CEP290 | Leber congenital amaurosis 10 |
| | CEP290 | Meckel syndrome 4 |
| | CEP290 | Senior-Loken syndrome 6 |
| | CLCNKB | Bartter syndrome, type 3 |
| | CLIC5 | Deafness, autosomal recessive 103 |
| | CLN6 | Ceroid lipofuscinosis, neuronal, 6A |
| | CLN6 | Ceroid lipofuscinosis, neuronal, 6B (Kufs type) |
| | CLN8 | Ceroid lipofuscinosis, neuronal, 8 |
| | CLN8 | Ceroid lipofuscinosis, neuronal, 8, Northern |
| | | epilepsy variant |
| | CLRN1 | Retinitis pigmentosa 61 |
| | CLRN1 | Usher syndrome, type 3A |
| | CNGA3 | Achromatopsia 2 |
| | COL11A1 | Fibrochondrogenesis 1 |
| | CPLANE1 | Joubert syndrome 17 |
| | CPLANE1 | Orofaciodigital syndrome VI |
| | CRX | Leber congenital amaurosis 7 |
| | CRYBB3 | Cataract 22 |
| | CYBA | Chronic granulomatous disease 4, autosomal |
| | | recessive |
| | DRAM2 | Cone-rod dystrophy 21 |
| | DYNC2H1 | Short-rib thoracic dysplasia 3 with or without |
| | | polydactyly |
| | GDF6 | Leber congenital amaurosis 17 |
| | GLRB | Hyperekplexia 2 |
| | GRM6 | Night blindness, congenital stationary |
| | | (complete), 1B, autosomal recessive |
| | HELLS | Immunodeficiency-centromeric instability- |
| | | facial anomalies syndrome 4 |
| | INPP5K | Muscular dystrophy, congenital, with cataracts |
| | | and intellectual disability |
| | ITGA8 | Renal hypodysplasia/aplasia 1 |
| | KCNJ10 | SESAME syndrome |
| | KCNMA1 | Cerebellar atrophy, developmental delay, and |
| | | seizures |
| | KLHL3 | Pseudohypoaldosteronism, type IID |
| | LAMC3 | Cortical malformations, occipital |
| | LRAT | Leber congenital amaurosis 14 |
| | LRAT | Retinal dystrophy, early-onset severe |
| | LRAT | Retinitis pigmentosa, juvenile |
| | LRIT3 | Night blindness, congenital stationary |
| | | (complete), 1F, autosomal recessive |
| | MMP9 | Metaphyseal anadysplasia 2 |
| | MYO3A | Deafness, autosomal recessive 30 |
| | MYO7A | Deafness, autosomal recessive 2 |
| | MYO7A | Usher syndrome, type 1B |
| | NEK1 | Short-rib thoracic dysplasia 6 with or without |
| | | polydactyly |
| | NPHP3 | Meckel syndrome 7 |
| | NPHP3 | Nephronophthisis 3 |
| | NPHP3 | Renal-hepatic-pancreatic dysplasia 1 |
| | NPR3 | Boudin-Mortier syndrome |
| | PCNT | Microcephalic osteodysplastic primordial |
| | | dwarfism, type II |
| | PDE6A | Retinitis pigmentosa 43 |
| | PDE6B | Retinitis pigmentosa-40 |
| | RD3 | Leber congenital amaurosis 12 |
| | RLBP1 | Bothnia retinal dystrophy |
| | RLBP1 | Newfoundland rod-cone dystrophy |
| | RPGRIP1L | COACH syndrome 3 |
| | RPGRIP1L | Joubert syndrome 7 |
| | RPGRIP1L | Meckel syndrome 5 |
| | SGPL1 | Nephrotic syndrome, type 14 |
| | SPATA7 | Leber congenital amaurosis 3 |
| | SPATA7 | Retinitis pigmentosa 94, variable age at onset, |
| | | autosomal recessive |
| | STAT1 | Immunodeficiency 31B, mycobacterial and |
| | | viral infections, autosomal recessive |
| | TACSTD2 | Corneal dystrophy, gelatinous drop-like |
| | TH | Segawa syndrome, recessive |
| | TMEM67 | COACH syndrome 1 |
| | TMEM67 | Joubert syndrome 6 |
| | TMEM67 | Meckel syndrome 3 |
| | TMEM67 | Nephronophthisis 11 |
| | TMEM67 | RHYNS syndrome |
| | TRPM1 | Night blindness, congenital stationary |
| | | (complete), 1C, autosomal recessive |
| | WNT2B | Diarrhea 9 |
| | XPNPEP3 | Nephronophthisis-like nephropathy 1 |
| Blood | AARS2 | Combined oxidative phosphorylation deficiency |
| | | 8 |
| | AARS2 | Leukoencephalopathy, progressive, with |
| | | ovarian failure |
| | ACADSB | 2-methylbutyrylglycinuria |
| | ACAT1 | Alpha-methylacetoacetic aciduria |
| | ADAR | Aicardi-Goutieres syndrome 6 |
| | ADARB1 | Neurodevelopmental disorder with hypotonia, |
| | | microcephaly, and seizures |
| | ADCY1 | Deafness, autosomal recessive 44 |
| | ADCY3 | Obesity, susceptibility to, BMIQ19} |
| | ADCY6 | Lethal congenital contracture syndrome 8 |
| | ADK | Hypermethioninemia due to adenosine kinase |
| | | deficiency |
| | ADPRS | Neurodegeneration, childhood-onset, stress- |
| | | induced, with variable ataxia and seizures |
| | AHSG | Alopecia-intellectual disability syndrome 1 |
| | AICDA | Immunodeficiency with hyper-IgM, type 2 |
| | AIMP1 | Leukodystrophy, hypomyelinating, 3 |
| | AIRE | Autoimmune polyendocrinopathy syndrome, |
| | | type I, with or without reversible metaphyseal |
| | | dysplasia |
| | AK7 | Spermatogenic failure 27 |
| | ALDOA | Glycogen storage disease XII |
| | ALDOB | Fructose intolerance, hereditary |
| | ALKBH8 | Intellectual developmental disorder, autosomal |
| | | recessive 71 |
| | AMPD1 | Myopathy due to myoadenylate deaminase |
| | | deficiency |
| | AMPD2 | Pontocerebellar hypoplasia, type 9 |
| | AMPD2 | Spastic paraplegia 63 |
| | ANAPC7 | Ferguson-Bonni neurodevelopmental syndrome |
| | ANO6 | Scott syndrome |
| | AP5Z1 | Spastic paraplegia 48, autosomal recessive |
| | APRT | Adenine phosphoribosyltransferase deficiency |
| | APTX | Ataxia, early-onset, with oculomotor apraxia |
| | | and hypoalbuminemia |
| | ASCC1 | Spinal muscular atrophy with congenital bone |
| | | fractures 2 |
| | ATP6V1A | Cutis laxa, autosomal recessive, type IID |
| | ATP6V1B1 | Distal renal tubular acidosis 2 with progressive |
| | | sensorineural hearing loss |
| | B3GALNT2 | Muscular dystrophy-dystroglycanopathy |
| | | (congenital with brain and eye anomalies, type |
| | | A, 11 |
| | BAAT | Bile acid conjugation defect 1 |
| | BLNK | Agammaglobulinemia 4 |
| | BLOC1S6 | Hermansky-Pudlak syndrome 9 |
| | BMPR1B | Acromesomelic dysplasia 3 |
| | BPGM | Erythrocytosis, familial, 8 |
| | BRIP1 | Fanconi anemia, complementation group J |
| | CANT1 | Desbuquois dysplasia 1 |
| | CANT1 | Epiphyseal dysplasia, multiple, 7 |
| | CARD11 | Immunodeficiency 11A |
| | CD19 | Immunodeficiency, common variable, 3 |
| | CD27 | Lymphoproliferative syndrome 2 |
| | CD40 | Immunodeficiency with hyper-IgM, type 3 |
| | CDC14A | Deafness, autosomal recessive 32, with or |
| | | without immotile sperm |
| | CDK6 | Microcephaly 12, primary, autosomal recessive |
| | CENPE | Microcephaly 13, primary, autosomal recessive |
| | CEP164 | Nephronophthisis 15 |
| | CFB | Complement factor B deficiency |
| | CHUK | Cocoon syndrome |
| | HUK | Popliteal pterygium syndrome, Bartsocas-Papas |
| | | type 2 |
| | CLCF1 | Cold-induced sweating syndrome 2 |
| | CNP | Leukodystrophy, hypomyelinating, 20 |
| | CNTNAP2 | Pitt-Hopkins like syndrome 1 |
| | COASY | Neurodegeneration with brain iron |
| | | accumulation 6 |
| | COASY | Pontocerebellar hypoplasia, type 12 |
| | CPS1 | Carbamoylphosphate synthetase I deficiency |
| | CSF2RB | Surfactant metabolism dysfunction, pulmonary, |
| | | 5 |
| | CSF3R | Neutropenia, severe congenital, 7, autosomal |
| | | recessive |
| | CSPP1 | Joubert syndrome 21 |
| | CTC1 | Cerebroretinal microangiopathy with |
| | | calcifications and cysts |
| | CTU2 | Microcephaly, facial dysmorphism, renal |
| | | agenesis, and ambiguous genitalia syndrome |
| | CXCR2 | WHIM syndrome 2 |
| | CYP19A1 | Aromatase deficiency |
| | CYP7B1 | Bile acid synthesis defect, congenital, 3 |
| | CYP7B1 | Spastic paraplegia 5A, autosomal recessive |
| | DALRD3 | Developmental and epileptic encephalopathy 86 |
| | DARS2 | Leukoencephalopathy with brain stem and |
| | | spinal cord involvement and lactate elevation |
| | DCT | Oculocutaneous albinism, type VIII |
| | DCXR | Pentosuria] |
| | DDC | Aromatic L-amino acid decarboxylase |
| | | deficiency |
| | DGUOK | Mitochondrial DNA depletion syndrome 3 |
| | | (hepatocerebral type) |
| | DGUOK | Progressive external ophthalmoplegia with |
| | | mitochondrial DNA deletions, autosomal |
| | | recessive 4 |
| | DHODH | Miller syndrome |
| | DLAT | Pyruvate dehydrogenase E2 deficiency |
| | DNASE1L3 | Systemic lupus erythematosus 16 |
| | DNMT3B | Immunodeficiency-centromeric instability- |
| | | facial anomalies syndrome 1 |
| | DOCK2 | Immunodeficiency 40 |
| | DPM1 | Congenital disorder of glycosylation, type Ie |
| | DPYD | 5-fluorouracil toxicity |
| | DPYD | Dihydropyrimidine dehydrogenase deficiency |
| | EDC3 | Intellectual developmental disorder, autosomal |
| | | recessive 50 |
| | ELAC2 | Combined oxidative phosphorylation deficiency |
| | | 17 |
| | ELP1 | Dysautonomia, familial |
| | ENTPD1 | Spastic paraplegia 64, autosomal recessive |
| | EPHB2 | Bleeding disorder, platelet-type, 22 |
| | EPX | Eosinophil peroxidase deficiency] |
| | ERBB2 | Visceral neuropathy, familial, 2, autosomal |
| | | recessive |
| | ERCC4 | Fanconi anemia, complementation group Q |
| | ERCC4 | XFE progeroid syndrome |
| | ERCC4 | Xeroderma pigmentosum, group F |
| | ERCC4 | Xeroderma pigmentosum, type F/Cockayne |
| | | syndrome |
| | ERCC5 | Cerebrooculofacioskeletal syndrome 3 |
| | ERCC5 | Xeroderma pigmentosum, group G |
| | ERCC5 | Xeroderma pigmentosum, group G/Cockayne |
| | | syndrome |
| | ERCC8 | Cockayne syndrome, type A |
| | ERCC8 | UV-sensitive syndrome 2 |
| | ESRP1 | Deafness, autosomal recessive 109 |
| | EXOSC1 | Pontocerebellar hypoplasia, type 1F |
| | F2 | Dysprothrombinemia |
| | F2 | Hypoprothrombinemia |
| | FADD | Immunodeficiency 90 with encephalopathy, |
| | | functional hyposplenia, and hepatic dysfunction |
| | FAM20C | Raine syndrome |
| | FANCD2 | Fanconi anemia, complementation group D2 |
| | FANCI | Fanconi anemia, complementation group I |
| | FANCL | Fanconi anemia, complementation group L |
| | FAR1 | Peroxisomal fatty acyl-CoA reductase 1 |
| | | disorder |
| | FARS2 | Combined oxidative phosphorylation deficiency |
| | | 14 |
| | FARS2 | Spastic paraplegia 77, autosomal recessive |
| | FARSA | Rajab interstitial lung disease with brain |
| | | calcifications 2 |
| | FASTKD2 | Combined oxidative phosphorylation deficiency |
| | | 44 |
| | FBXO7 | Parkinson disease 15, autosomal recessive |
| | FERMT3 | Leukocyte adhesion deficiency, type III |
| | FITM2 | Siddiqi syndrome |
| | FOXE3 | Anterior segment dysgenesis 2, multiple |
| | | subtypes |
| | FOXN1 | T-cell immunodeficiency, congenital alopecia, |
| | | and nail dystrophy |
| | GATB | Combined oxidative phosphorylation deficiency |
| | | 41 |
| | GCDH | Glutaricaciduria, type I |
| | GCK | Diabetes mellitus, permanent neonatal 1 |
| | GFI1B | Bleeding disorder, platelet-type, 17 |
| | GNE | Nonaka myopathy |
| | GPD1 | Hypertriglyceridemia, transient infantile |
| | GPSM2 | Chudley-Mccullough syndrome |
| | GTPBP2 | Jaberi-Elahi syndrome |
| | GTPBP3 | Combined oxidative phosphorylation deficiency |
| | | 23 |
| | HAAO | Vertebral, cardiac, renal, and limb defects |
| | | syndrome 1 |
| | HAVCR2 | T-cell lymphoma, subcutaneous panniculitis- |
| | | like |
| | HELLS | Immunodeficiency-centromeric instability- |
| | | facial anomalies syndrome 4 |
| | HJV | Hemochromatosis, type 2A |
| | HMOX1 | Heme oxygenase-1 deficiency |
| | HPCA | Dystonia 2, torsion, autosomal recessive |
| | HSD17B4 | D-bifunctional protein deficiency |
| | HSD17B4 | Perrault syndrome 1 |
| | HSF2BP | Premature ovarian failure 19 |
| | IFNGR1 | Immunodeficiency 27A, mycobacteriosis, AR |
| | IGHMBP2 | Charcot-Marie-Tooth disease, axonal, type 2S |
| | IGHMBP2 | Neuronopathy, distal hereditary motor, type VI |
| | IKBKB | Immunodeficiency 15B |
| | IL12B | Immunodeficiency 29, mycobacteriosis |
| | IL12RB1 | Immunodeficiency 30 |
| | IL17RC | Candidiasis, familial, 9 |
| | IL21 | Immunodeficiency, common variable, 11 |
| | IL21R | Immunodeficiency 56 |
| | IL2RA | Immunodeficiency 41 with lymphoproliferation |
| | | and autoimmunity |
| | IL2RB | Immunodeficiency 63 with lymphoproliferation |
| | | and autoimmunity |
| | IRF7 | Immunodeficiency 39 |
| | IRF8 | Immunodeficiency 32B, monocyte and |
| | | dendritic cell deficiency, autosomal recessive |
| | ITCH | Autoimmune disease, multisystem, with facial |
| | | dysmorphism |
| | ITK | Lymphoproliferative syndrome 1 |
| | JAG2 | Muscular dystrophy, limb-girdle, autosomal |
| | | recessive 27 |
| | JAK3 | SCID, autosomal recessive, T-negative/B- |
| | | positive type |
| | JAM2 | Basal ganglia calcification, idiopathic, 8, |
| | | autosomal recessive |
| | JAM3 | Hemorrhagic destruction of the brain, |
| | | subependymal calcification, and cataracts |
| | KYNU | Hydroxykynureninuria |
| | KYNU | Vertebral, cardiac, renal, and limb defects |
| | | syndrome 2 |
| | LAT | Immunodeficiency 52 |
| | LCP2 | Immunodeficiency 81 |
| | LEP | Obesity, morbid, due to leptin deficiency |
| | LEPR | Obesity, morbid, due to leptin receptor |
| | | deficiency |
| | LIPE | Lipodystrophy, familial partial, type 6 |
| | LYST | Chediak-Higashi syndrome |
| | MCCC2 | 3-Methylcrotonyl-CoA carboxylase 2 |
| | | deficiency |
| | MED25 | Basel-Vanagait-Smirin-Yosef syndrome |
| | METTL5 | Intellectual developmental disorder, autosomal |
| | | recessive 72 |
| | MGME1 | Mitochondrial DNA depletion syndrome 11 |
| | MLH1 | Mismatch repair cancer syndrome 1 |
| | MMP9 | Metaphyseal anadysplasia 2 |
| | MOCOS | Xanthinuria, type II |
| | MOCS2 | Molybdenum cofactor deficiency B |
| | MPIG6B | Thrombocytopenia, anemia, and myelofibrosis |
| | MPZL2 | Deafness, autosomal recessive 111 |
| | MSH3 | Familial adenomatous polyposis 4 |
| | MSH5 | Premature ovarian failure 13 |
| | NADSYN1 | Vertebral, cardiac, renal, and limb defects |
| | | syndrome 3 |
| | NAXE | Encephalopathy, progressive, early-onset, with |
| | | brain edema and/or leukoencephalopathy |
| | NBAS | Infantile liver failure syndrome 2 |
| | NBAS | Short stature, optic nerve atrophy, and Pelger- |
| | | Huet anomaly |
| | NCAPD3 | Microcephaly 22, primary, autosomal recessive |
| | NCF1 | Chronic granulomatous disease 1, autosomal |
| | | recessive |
| | NCF2 | Chronic granulomatous disease 2, autosomal |
| | | recessive |
| | NDRG1 | Charcot-Marie-Tooth disease, type 4D |
| | NDUFA10 | Mitochondrial complex I deficiency, nuclear |
| | | type 22 |
| | NDUFA8 | Mitochondrial complex I deficiency, nuclear |
| | | type 37 |
| | NDUFB8 | Mitochondrial complex I deficiency, nuclear |
| | | type 32 |
| | NDUFB9 | Mitochondrial complex I deficiency, nuclear |
| | | type 24 |
| | NDUFS1 | Mitochondrial complex I deficiency, nuclear |
| | | type 5 |
| | NHP2 | Dyskeratosis congenita, autosomal recessive 2 |
| | | Glucocorticoid deficiency 4, with or without |
| | NNT | mineralocorticoid deficiency |
| | NPR2 | Acromesomelic dysplasia 1, Maroteaux type |
| | NPR3 | Boudin-Mortier syndrome |
| | NT5C2 | Spastic paraplegia 45, autosomal recessive |
| | NTRK1 | Insensitivity to pain, congenital, with anhidrosis |
| | NUP62 | Striatonigral degeneration, infantile |
| | NUP85 | Nephrotic syndrome, type 17 |
| | OGDH | Oxoglutarate dehydrogenase deficiency |
| | PAH | Hyperphenylalaninemia, non-PKU mild] |
| | PAH | Phenylketonuria |
| | PARK7 | Parkinson disease 7, autosomal recessive early- |
| | | onset |
| | PAX7 | Myopathy, congenital, progressive, with |
| | | scoliosis |
| | PDE2A | Intellectual developmental disorder with |
| | | paroxysmal dyskinesia or seizures |
| | PDHX | Lacticacidemia due to PDX1 deficiency |
| | PDX1 | Pancreatic agenesis 1 |
| | PDXK | Neuropathy, hereditary motor and sensory, type |
| | | VIC, with optic atrophy |
| | PFKM | Glycogen storage disease VII |
| | PGM3 | Immunodeficiency 23 |
| | PKDCC | Rhizomelic limb shortening with dysmorphic |
| | | features |
| | PLCB1 | Developmental and epileptic encephalopathy 12 |
| | PLEKHM1 | Osteopetrosis, autosomal recessive 6 |
| | POC1A | Short stature, onychodysplasia, facial |
| | | dysmorphism, and hypotrichosis |
| | POLG2 | Mitochondrial DNA depletion syndrome 16 |
| | | (hepatic type) |
| | POLG2 | Mitochondrial DNA depletion syndrome 16B |
| | | (neuroophthalmic type) |
| | PRF1 | Hemophagocytic lymphohistiocytosis, familial, |
| | | 2 |
| | PRKDC | Immunodeficiency 26, with or without |
| | | neurologic abnormalities |
| | PRKRA | Dystonia 16 |
| | PTH | Hypoparathyroidism, familial isolated 1 |
| | PTS | Hyperphenylalaninemia, BH4-deficient, A |
| | PUS1 | Myopathy, lactic acidosis, and sideroblastic |
| | | anemia 1 |
| | PUS7 | Intellectual developmental disorder with |
| | | abnormal behavior, microcephaly, and short |
| | | stature |
| | PYCR2 | Leukodystrophy, hypomyelinating, 10 |
| | QRSL1 | Combined oxidative phosphorylation deficiency |
| | | 40 |
| | RAB23 | Carpenter syndrome |
| | RAC2 | Immunodeficiency 73C with defective |
| | | neutrophil chemotaxis and |
| | | hypogammaglobulinemia |
| | RD3 | Leber congenital amaurosis 12 |
| | RDX | Deafness, autosomal recessive 24 |
| | RNASEH2C | Aicardi-Goutieres syndrome 3 |
| | RNF168 | RIDDLE syndrome |
| | ROR2 | Robinow syndrome, autosomal recessive |
| | RPIA | Ribose 5-phosphate isomerase deficiency |
| | SDHB | Mitochondrial complex II deficiency, nuclear |
| | | type 4 |
| | SETX | Spinocerebellar ataxia, autosomal recessive, |
| | | with axonal neuropathy 2 |
| | SHMT2 | Neurodevelopmental disorder with |
| | | cardiomyopathy, spasticity, and brain |
| | | abnormalities |
| | SLC24A5 | Albinism, oculocutaneous, type VI |
| | SLC24A5 | Skin/hair/eye pigmentation 4, fair/dark skin] |
| | SLC25A13 | Citrullinemia, adult-onset type II |
| | SLC25A13 | Citrullinemia, type II, neonatal-onset |
| | SLC39A8 | Congenital disorder of glycosylation, type IIn |
| | SLC6A3 | Parkinsonism-dystonia, infantile, 1 |
| | SLC9A1 | Lichtenstein-Knorr syndrome |
| | SMG9 | Heart and brain malformation syndrome |
| | SMOC2 | Dentin dysplasia, type I, with microdontia and |
| | | misshapen teeth |
| | SNX10 | Osteopetrosis, autosomal recessive 8 |
| | SPIDR | Ovarian dysgenesis 9 |
| | SPNS2 | Deafness, autosomal recessive 115 |
| | SPR | Dystonia, dopa-responsive, due to sepiapterin |
| | | reductase deficiency |
| | SPTA1 | Pyropoikilocytosis |
| | SPTA1 | Spherocytosis, type 3 |
| | | Immunodeficiency 31B, mycobacterial and |
| | STAT1 | viral infections, autosomal recessive |
| | | Hemophagocytic lymphohistiocytosis, familial, |
| | STX11 | 4 |
| | TAF2 | Intellectual developmental disorder, autosomal |
| | | recessive 40 |
| | TDO2 | ?Hypertryptophanemia] |
| | TDP1 | Spinocerebellar ataxia, autosomal recessive, |
| | | with axonal neuropathy 1 |
| | TENT5A | Osteogenesis imperfecta, type XVIII |
| | TGDS | Catel-Manzke syndrome |
| | TH | Segawa syndrome, recessive |
| | THG1L | Spinocerebellar ataxia, autosomal recessive 28 |
| | TNFRSF11B | Paget disease of bone 5, juvenile-onset |
| | TRIP13 | Mosaic variegated aneuploidy syndrome 3 |
| | TRIP13 | Oocyte maturation defect 9 |
| | TRMT1 | Intellectual developmental disorder, autosomal |
| | | recessive 68 |
| | TYK2 | Immunodeficiency 35 |
| | UPB 1 | Beta-ureidopropionase deficiency |
| | USP45 | Leber congenital amaurosis 19 |
| | VARS2 | Combined oxidative phosphorylation deficiency |
| | | 20 |
| | WDR4 | Galloway-Mowat syndrome 6 |
| | WDR4 | Microcephaly, growth deficiency, seizures, and |
| | | brain malformations |
| | WNT1 | Osteogenesis imperfecta, type XV |
| | WNT10B | Split-hand/foot malformation 6 |
| | WRAP53 | Dyskeratosis congenita, autosomal recessive 3 |
| | XDH | Xanthinuria, type I |
| | ZFYVE26 | Spastic paraplegia 15, autosomal recessive |

As disclosed herein, modified capsids as disclosed herein may be transported across the blood brain barrier and used to infect cells of the central nervous system. Thus, such modified capsids may be useful for the transport of a nucleotide of interest across the blood brain barrier for the gene therapy of a brain disorder, e.g., a disorder of the central nervous system (CNS), a disorder with neurological symptoms, etc. In such cases, expression of a therapeutic gene may be limited to cells central nervous system, e.g., neurons, by operably linking the nucleotide of interest to a tissue specific promoter. As a non-limiting example, neuron specific promoters include but are not limited to Syn1, NSE, and MeCP2. Non-limiting examples of oligodendrocyte promoters include, but are not limited to, MBP and MAG. Non-limiting examples of microglia specific promoters include, but are not limited to, CD68, HEXB, and F4/80. Non-limiting examples of astrocyte specific promoters include, but aren't limited to, GFAP and ALDH1L1. In certain embodiments, the promoter is brain-specific (e.g., neuron-specific, glial cell-specific, astrocyte-specific, oligodendrocyte-specific, microglia-specific and/or central nervous system-specific). Exemplary brain-specific promoters may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. Other examples of such brain-specific promoter include, but are not limited to, SCG10, tubulin al promoter, calcium/calmodulin-dependent protein kinase II (CaMKII) promoter, neuron-specific enolase (NSE) promoter, PDGF (platelet-derived growth factor beta)-b chain promoter and the like. More brain-specific promoter elements are disclosed in WO 2016/100575A1, which is incorporated by reference herein in its entirety.

In some embodiments, a brain-specific promoter as described herein is selected from the group consisting of a Synapsin 1 promoter, a Calcium/calmodulin-dependent protein kinase II (CaMKII) promoter, a tyrosine hydroxylase (TH) promoter, a Forkhead Box A2 (FOXA2) promoter, an alpha-internexin (INA) promoter, a Nestin (NES) promoter, a Glial fibrillary acidic protein (GFAP) promoter, an Aldehyde Dehydrogenase 1 Family Member L1 (ALDH1L1) promoter, a myelin-associated oligodendrocyte basic protein (MOBP) promoter and a Myelin basic protein (MBP) promoter.

In other embodiments, the promoter is a neuron-, astrocyte-, or oligodendrocyte-specific or neuron-, astrocyte-, or oligodendrocyte-preferential promoter, for example, a Synapsin, a MeCP2, an oligodendrocyte transcription factor 1 (Olig1), a chondroitin sulfate proteoglycan (Cspg4), or a CNP (2′,3′-Cyclic-nucleotide 3′-phosphodiesterase) promoter.

CNS disorders and disorders with neurological symptoms amenable to gene therapy include, but are not limited to: Alzheimer's, brain cancer, Behcet's Disease, cerebral Lupus, Creutzfeldt-Jakob Disease, dementia, epilepsy, encephalitis, Friedreich's Ataxia, Guillain-Barre Syndrome, Gaucher Disease, headache, hydrocephalus, Huntington's disease, intracranial hypertension, leukodystrophy, migraine, myasthenia gravis, muscular dystrophy, multiple sclerosis, narcolepsy, neuropathy, Prader-Willi Syndrome, Parkinson's disease, Rett Syndrome, restless leg syndrome, sleep disorders, subarachnoid haemorrhage, stroke, traumatic brain injury, trigeminal neuralgia, transient ischaemic attack, and Von Hippel-Lindau Syndrome (angiomatosis).

In some embodiments, a viral capsid as described herein may encapsidate a therapeutic gene in which the expression prevents, alleviates, or otherwise reduces a one or more symptoms of an enzyme-deficiency disease and/or a disease selected from the group consisting of Fabry disease, Gaucher disease, MPS I, MPS II, MPS IIIA, MPS IIIB, MPS IIID, MIPS IVB, MPS VI, MPS VII, MPS IX, Pompe disease, Lysosomal acid lipase deficiency, Metachromatic leukodystrophy, Niemann-Pick diseases types A, B, and C2, Alpha mannosidosis, Neuraminidase deficiency, Sialidosis, Aspartylglycosaminuria, Combined saposin deficiency, Atypical Gaucher disease, Farber lipogranulomatosis, Fucosidosis, and Beta mannosidosis.

“Enzyme-deficiency diseases” include non-lysosomal storage disease such as Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, and lysosomal storage diseases. “Lysosomal storage diseases” include any disorder resulting from a defect in lysosome function. Currently, approximately 50 lysosomal storage disorders have been identified, the most well-known of which include Tay-Sachs, Gaucher, and Niemann-Pick disease. The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect. Thus, as used herein, the term “enzyme” is meant to encompass those other proteins associated with lysosomal storage diseases.

The nature of the molecular lesion affects the severity of the disease in many cases, i.e. complete loss-of-function tends to be associated with pre-natal or neo-natal onset, and involves severe symptoms; partial loss-of-function is associated with milder (relatively) and later-onset disease. Generally, only a small percentage of activity needs to be restored to have to correct metabolic defects in deficient cells. Lysosomal storage diseases are generally described in Desnick and Schuchman, 2012.

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Options for the treatment of lysosomal storage diseases include enzyme replacement therapy (ERT), substrate reduction therapy, pharmacological chaperone-mediated therapy, hematopoietic stem cell transplant therapy, and gene therapy. An example of substrate reduction therapy includes the use of Miglustat or Eliglustat to treat Gaucher Type 1. These drugs act by blocking synthase activity, which reduces subsequent substrate production. Hematopoietic stem cell therapy (HSCT), for example, is used to ameliorate and slow-down the negative central nervous system phenotype in patients with some forms of MPS. See R. M. Boustany, “Lysosomal storage diseases—the horizon expands,” 9(10) Nat. Rev. Neurol. 583-98, October 2013; which reference is incorporated herein in its entirety by reference.

Two of the most common LSDs are Pompe disease and Fabry disease. Pompe disease, which has an estimated incidence of 1 in 10,000, is caused by defective lysosomal enzyme alpha-glucosidase (GAA), which results in the deficient processing of lysosomal glycogen. Accumulation of lysosomal glycogen occurs predominantly in skeletal, cardiac, and hepatic tissues. Infantile onset Pompe causes cardiomegaly, hypotonia, hepatomegaly, and death due to cardiorespiratory failure, usually before 2 years of age. Adult onset Pompe occurs as late as the second to sixth decade and usually involves only skeletal muscle. Treatments currently available include Genzyme's MYOZYME®/LUMIZYME® (alglucosidase alfa), which is a recombinant human alpha-glucosidase produced in CHO cells and administered by intravenous infusion.

Fabry disease, which has including mild late onset cases an overall estimated incidence of 1 in 3,000, is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments currently available include Genzyme's FABRAZYME® (agalsidase beta), which is a recombinant human alpha-galactosidase A produced in CHO cells and administered by intravenous infusion; Shire's REPLAGAL™ (agalsidase alfa), which is a recombinant human alpha-galactosidase A produced in human fibroblast cells and administered by intravenous infusion; and Amicus's GALAFOLD™ (migalastat or 1-deoxygalactonojirimycin) an orally administered small molecule chaperone that shifts the folding of abnormal alpha-galactosidase A to a functional conformation.

A further embodiment of the present invention is a process for the preparation of a modified capsid protein, the method comprising the steps of:
a) expressing a nucleic acid encoding the modified capsid protein under suitable conditions, and
b) isolating the expressed capsid protein of step a).

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. A method for making such a mosaic viral particle comprises:
a) expressing a nucleic acid encoding the modified capsid protein and a nucleotide encoding a reference capsid protein at a ratio (wt/wt) of at least about 60:1 to about 1:60, e.g., 2:1, 1:1, 3:5, 1:2, 1:3, etc. under suitable conditions, and
b) isolating the expressed capsid protein of step a).

In some embodiments, a composition described herein comprises, or a method described herein combines, a modified cap gene: reference cap gene (or combination of reference cap genes) at a ratio that ranges from at least about 1:60 to about 60:1, e.g., 2:1, 1:1, 3:5, 1:2, 1:3, etc. In some embodiments, the ratio is at least about 1:2. In some embodiments, the ratio is at least about 1:3. In some embodiments, the ratio is at least about 1:4. In some embodiments, the ratio is at least about 1:5. In some embodiments, the ratio is at least about 1:6. In some embodiments, the ratio is at least about 1:7. In some embodiments, the ratio is at least about 1:8. In some embodiments, the ratio is at least about 1:9. In some embodiments, the ratio is at least about 1:10. In some embodiments, the ratio is at least about 1:11. In some embodiments, the ratio is at least about 1:12. In some embodiments, the ratio is at least about 1:13. In some embodiments, the ratio is at least about 1:14. In some embodiments, the ratio is at least about 1:15. In some embodiments, the ratio is at least about 1:16. In some embodiments, the ratio is at least about 1:17. In some embodiments, the ratio is at least about 1:18. In some embodiments, the ratio is at least about 1:19. In some embodiments, the ratio is at least about 1:20. In some embodiments, the ratio is at least about 1:25. In some embodiments, the ratio is at least about 1:30. In some embodiments, the ratio is at least about 1:35. In some embodiments, the ratio is at least about 1:40. In some embodiments, the ratio is at least about 1:45. In some embodiments, the ratio is at least about 1:50. In some embodiments, the ratio is at least about 1:55. In some embodiments, the ratio is at least about 1:60. In some embodiments, the ratio is at least about 2:1. In some embodiments, the ratio is at least about 3:1. In some embodiments, the ratio is at least about 4:1. In some embodiments, the ratio is at least about 5:1. In some embodiments, the ratio is at least about 6:1. In some embodiments, the ratio is at least about 7:1. In some embodiments, the ratio is at least about 8:1. In some embodiments, the ratio is at least about 9:1. In some embodiments, the ratio is at least about 10:1. In some embodiments, the ratio is at least about 11:1. In some embodiments, the ratio is at least about 12:1. In some embodiments, the ratio is at least about 13:1. In some embodiments, the ratio is at least about 14:1. In some embodiments, the ratio is at least about 15:1. In some embodiments, the ratio is at least about 16:1. In some embodiments, the ratio is at least about 17:1. In some embodiments, the ratio is at least about 18:1. In some embodiments, the ratio is at least about 19:1. In some embodiments, the ratio is at least about 20:1. In some embodiments, the ratio is at least about 25:1. In some embodiments, the ratio is at least about 30:1. In some embodiments, the ratio is at least about 35:1. In some embodiments, the ratio is at least about 40:1. In some embodiments, the ratio is at least about 45:1. In some embodiments, the ratio is at least about 50:1. In some embodiments, the ratio is at least about 55:1. In some embodiments, the ratio is at least about 60:1.

In some embodiments, VP protein subunit ratios in the mosaic viral particle may, but do not necessarily, stoichiometrically reflect the ratios of modified cap gene:reference cap gene. As a non-limiting exemplary embodiment, a mosaic capsid formed according to the method may be considered to, but does not necessarily, have a modified capsid protein:reference capsid protein ratio similar to the ratio (wt:wt) of nucleic acids encoding same used to produce the mosaic capsid. In some embodiments, a mosaic capsid comprises a protein subunit ratio of about 1:59 to about 59:1. In some embodiments, a mosaic capsid comprises a modified capsid protein:reference capsid protein ratio of about 7:1.

Further embodiments of the present invention is a method for altering the tropism of a virus, the method comprising the steps of (a) inserting a nucleic acid encoding an amino acid sequence into a nucleic acid sequence encoding an viral capsid protein to form a nucleotide sequence encoding a genetically modified capsid protein comprising the amino acid sequence and/or (b) culturing a packaging cell in conditions sufficient for the production of viral particles, wherein the packaging cell comprises the nucleic acid. A further embodiment of the present invention is a method for displaying a targeting ligand on the surface of a capsid protein, the method comprising the steps of: (a) expressing a nucleic acid encoding a modified viral capsid protein as described herein (and optionally with a nucleotide encoding a reference capsid protein) under suitable conditions, wherein the nucleic acid encodes a capsid protein comprising a first member of a specific binding pair, (b) isolating the expressed capsid protein comprising a first member of a specific binding pair of step (a) or a capsid comprising same, and (c) incubating the capsid protein or capsid with a second cognate member of the specific binding pair under conditions suitable for allowing the formation of an isopeptide bond between the first and second member, wherein the second cognate member of the specific binding pair is fused with a targeting ligand.

In some embodiments, the packaging cell further comprises a helper plasmid and/or a transfer plasmid comprising a nucleotide of interest. In some embodiments, the methods further comprise isolating self-complementary adeno-associated viral particles from culture supernatant. In some embodiments, the methods further comprise lysing the packaging cell and isolating single-stranded adeno-associated viral particles from the cell lysate. In some embodiments, the methods further comprise (a) clearing cell debris, (b) treating the supernatant containing viral particles with nucleases, e.g., DNase I and MgCl₂, (c) concentrating viral particles, (d) purifying the viral particles, and (e) any combination of (a)-(d).

Packaging cells useful for production of the viral particles described herein include, e.g., animal cells permissive for the virus, or cells modified to be permissive for the virus; or the packaging cell construct, for example, with the use of a transformation agent such as calcium phosphate. Non-limiting examples of packaging cell lines useful for producing viral particles described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raji (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J Virol 69, 7430-7436, NSO (murine myeloma) cells, human amniocytic cells (e.g., CAP, CAP-T), yeast cells (including, but not limited to, S. cerevisiae, Pichia pastoris), plant cells (including, but not limited to, Tobacco NT1, BY-2), insect cells (including but not limited to SF9, S2, SF21, Tni (e.g. High 5)) or bacterial cells (including, but not limited to, E. coli).

For additional packaging cells and systems, packaging techniques and particles for packaging the nucleic acid genome into the pseudotyped viral particle see, for example, Polo, et al, Proc Natl Acad Sci USA, (1999) 96:4598-4603. Methods of packaging include using packaging cells that permanently express the viral components, or by transiently transfecting cells with plasmids.

Further embodiments include methods of redirecting a virus and/or delivering a reporter or therapeutic gene to a target cell, the method comprising a method for transducing cells in vitro (e.g., ex vivo) or in vivo, the method comprising the steps of: contacting the target cell with a viral particle comprising a capsid described herein, wherein the capsid comprises a targeting ligand that specifically binds a receptor expressed by the target cell. In some embodiments, the target cell is in vitro (e.g., ex vivo). In other embodiments, the target cell is in vivo in a subject, e.g., a human.

### Target Cells

A wide variety of cells may be targeted in order to deliver a nucleotide of interest using a modified viral particle as disclosed herein. The target cells will generally be chosen based upon the nucleotide of interest and the desired effect.

In some embodiments, a nucleotide of interest may be delivered to enable a target cell to produce a protein that makes up for a deficiency in an organism, such as an enzymatic deficiency, or immune deficiency, such as X-linked severe combined immunodeficiency. Thus, in some embodiments, cells that would normally produce the protein in the animal are targeted. In other embodiments, cells in the area in which a protein would be most beneficial are targeted.

In other embodiments, a nucleotide of interest, such as a gene encoding an siRNA, may inhibit expression of a particular gene in a target cell. The nucleotide of interest may, for example, inhibit expression of a gene involved in a pathogen life cycle. Thus, cells susceptible to infection from the pathogen or infected with the pathogen may be targeted. In other embodiments, a nucleotide of interest may inhibit expression of a gene that is responsible for production of a toxin in a target cell.

In other embodiments a nucleotide of interest may encode a toxic protein that kills cells in which it is expressed. In this case, tumor cells or other unwanted cells may be targeted.

In still other embodiments a nucleotide of interest that encodes a therapeutic protein. In some embodiments, the nucleotide of interest encodes a therapeutic protein that may secreted from a transduced cell, and that may provide a therapeutic effect in the interstitial space surrounding the transduced cell or on a neighboring cell. In some embodiments, the nucleotide of interest encodes a therapeutic protein that may provide a therapeutic effect in an autonomous manner to the transduced cell. In some embodiments, the nucleotide of interest encodes a therapeutic protein that may provide a therapeutic benefit in an autonomous manner to the transduced cell, within the interstitial space surrounding the transduced cell, and/or to a cell neighboring the transduced cell.

Once a particular population of target cells is identified in which expression of a nucleotide of interest is desired, a target receptor is selected that is specifically expressed on that population of target cells. The target receptor may be expressed exclusively on that population of cells or to a greater extent on that population of cells than on other populations of cells. The more specific the expression, the more specifically delivery can be directed to the target cells. Depending on the context, the desired amount of specificity of the marker (and thus of the gene delivery) may vary. For example, for introduction of a toxic gene, a high specificity is most preferred to avoid killing non-targeted cells. For expression of a protein for harvest, or expression of a secreted product where a global impact is desired, less marker specificity may be needed.

As discussed above, the target receptor may be any receptor for which a targeting ligand can be identified or created. Preferably the target receptor is a peptide or polypeptide, such as a receptor. However, in other embodiments the target receptor may be a carbohydrate or other molecule that can be recognized by a binding partner. If a binding partner, e.g., ligand, for the target receptor is already known, it may be used as the affinity molecule. However, if a binding molecule is not known, antibodies to the target receptor may be generated using standard procedures. The antibodies can then be used as a targeting ligand.

Thus, target cells may be chosen based on a variety of factors, including, for example, (1) the application (e.g., therapy, expression of a protein to be collected, and conferring disease resistance) and (2) expression of a marker with the desired amount of specificity.

Target cells are not limited in any way and include both germline cells and cell lines and somatic cells and cell lines. Target cells can be stem cells derived from either origin. When the target cells are germline cells, the target cells are preferably selected from the group consisting of single-cell embryos and embryonic stem cells (ES).

### Pharmaceutical Compositions, Dosage Forms and Administration

A further embodiment provides a medicament comprising at least one modified viral capsid protein and appropriate targeting ligand according to this invention and/or a nucleic acid according to this invention. Preferably such medicament is useful as a gene transfer particle.

Also disclosed herein are pharmaceutical compositions comprising the viral particles described herein and a pharmaceutically acceptable carrier and/or excipient. In addition, disclosed herein are pharmaceutical dosage forms comprising the viral particle described herein.

As discussed herein, the viral particles described herein can be used for various therapeutic applications (in vivo and ex vivo) and as research tools.

Pharmaceutical compositions based on the viral particles disclosed herein can be formulated in any conventional manner using one or more physiologically acceptable carriers and/or excipients. The viral particles may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical or localized administration. Techniques and formulations can be found in, for example, Remington's Pharmaceutical Sciences, Meade Publishing Co., Easton, Pa. For systemic administration, injection is preferred, including intracerebroventricular, intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g., by bolus injection or continuous infusion. Formulations for injection can be presented in a unit dosage form, e.g., in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the viral particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered viral particles described herein on a daily or weekly basis for a time period or on a monthly, bi-yearly or yearly basis depending on need or exposure to a pathogenic organism or to a condition in the subject (e.g., cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the viral particles and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal or anti-bacterial agents or anti-tumor agents may be needed for compositions described.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Any method known to one skilled in the art maybe used for large scale production of viral particles, packaging cells and particle constructs described herein. For example, master and working seed stocks may be prepared under GMP conditions in qualified primary CEFs or by other methods. Packaging cells may be plated on large surface area flasks, grown to near confluence and viral particles purified. Cells may be harvested and viral particles released into the culture media isolated and purified, or intracellular viral particles released by mechanical disruption (cell debris can be removed by large-pore depth filtration and host cell DNA digested with endonuclease). Virus particles may be subsequently purified and concentrated by tangential-flow filtration, followed by diafiltration. The resulting concentrated bulk maybe formulated by dilution with a buffer containing stabilizers, filled into vials, and lyophilized. Compositions and formulations may be stored for later use. For use, lyophilized viral particles may be reconstituted by addition of diluent.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

TABLE 5

| | | | | |
|---|---|---|---|---|
| | | | Region | Region |
| | | | encoding the | encoding the |
| | | | non-structural/ | capsid protein |
| | | | rep protein | (Accession |
| | | | (Accession | Number/ |
| | Accession | | Number/ | GI |
| | Number/ | | GI Number of | Number of |
| Description | GI Number | ITR | protein) | protein) |

Avian adeno-associated virus

| | | | | |
|---|---|---|---|---|
| Avian adeno-associated | GQ368252.1/ | | 243-2237 | 2255-4471 |
| virus isolate YZ-1, | GI: | | (ACU30841.1/ | (ACU30842.1/ |
| complete genome | 255710032 | | GI: 255710033) | GI: 255710034) |
| Avian adeno-associated | KF937794.1/ | 1-142 | 244-2235 | 2253-4469 |
| viris isolate ZN1, | GI: | 4540-4682 | (AHK22792.1/ | (AHK22793.1/ |
| complete genome | 588284633 | | GI: 588284634) | GI: 588284635) |

Avian adeno-associated virus ATCC VR-865

| | | | | |
|---|---|---|---|---|
| Avian adeno-associated | AY186198.1/ | 1-142 | 244-2232 | 2250-4481 |
| virus ATCC VR-865, | GI: 31414777 | 4552-4694 | (AAO32086.1/ | (AAO32087.1/ |
| complete genome | | | GI: 31414778) | GI: 31414779) |
| Avian adeno-associated | NC_004828.1/ | 1-142 | 244-2232 | 2250-4481 |
| virus ATCC VR-865, | GI: 31543992 | 4552-4694 | (NP_852780.1/ | (NP_852781.1/ |
| complete genome | | | GI: 31543993) | GI: 31543994) |
| Avian adeno-associated | AY629582.3/ | 1-142 | 244-2232 | 2250-4481 |
| virus ATCC VR-865, | GI: 48996102 | 4552-4694 | (AAT48612.1/ | (AAT48613.1/ |
| complete genome | | | GI: 48996103) | GI: 48996104) |

Avian adeno-associated virus strain DA-1

| | | | | |
|---|---|---|---|---|
| Avian adeno-associated | AY629583.1/ | 1-142 | 244-2235 | 2253-4469 |
| virus strain DA-1, | GI: 48996105 | 4540-1682 | (AAT48614.1/ | (AAT48615.1/ |
| complete genome | | | GI: 48996106) | GI: 48996107) |
| Avian adeno-associated | NC_006263.1/ | 1-142 | 244-2235 | 2253-4469 |
| virus strain DA-1, | GI: 51949968 | 4540-4682 | (YP_077182.1/ | (YP_077183.1/ |
| complete genome | | | GI: 51949969) | GI: 51949970) |

Bat adeno-associated virus YNM

| | | | |
|---|---|---|---|
| Bat adeno-associated | GU226971.1/ | 200-2041 | 2059-4233 |
| virus YNM, complete | GI: 289719008 | (ADD17085.1/ | (ADD17086.1/ |
| sequence | | GI: 289719009) | GI: 289719010) |
| Bat adeno-associated | NC_014468.1/ | 200-2041 | 2059-4233 |
| virus YNM, complete | GI: 304422916 | (YP_003858571.1/ | (YP_003858572.1/ |
| genome | | GI: 304422917) | GI: 304422918) |

Bovine adeno-associated virus

| | | | |
|---|---|---|---|
| Bovine adeno- | AY388617.1/ | 368-2200 | 2216-4426 |
| associated virus, | GI: 38679253 | (AAR26464.1/ | (AAR26465.1/ |
| complete genome | | GI: 38679254) | GI: 38679255) |
| Bovine adeno- | NC_0058889.1/ | 368-2200 | 2216-4426 |
| associated virus, | GI: 48696557 | (YP_024970.1/ | (YP_024971.1/ |
| complete genome | | GI: 48696558) | GI: 48696559) |

Bovine parvovirus-2

| | | | |
|---|---|---|---|
| Bovine parvovirus 2 | AF406966.1/ | 306-1919 | 2268-5384 |
| putative non-structural | GI: 15825370 | (AAL09671.1/ | (AAL09672.1/ |
| protein and putative | | GI: 15825371) | GI: 15825372) |
| capsid protein genes, | | | |
| complete cds. | | | |
| Bovine parvovirus 2, | NC_006259.1/ | 306-1919 | 2268-5384 |
| complete genome | GI: 51949957 | (YP_077175.1/ | (YP_077176.1/ |
| | | GI: 51949958) | GI: 51949959) |

California sea lion adeno-associated virus 1

| | | | |
|---|---|---|---|
| California sea lion | JN420371.1/ | 284-2086 | 2117-4273 |
| adeno-associated virus | GI: 343458966 | (AEM37641.1/ | (AEM37642.1/ |
| 1 isolate I136 Rep78 | | GI: 343458967) | GI: 343458968) |
| and VP1genes, | | | |
| complete cds. | | | |
| California sea lion | JN420372.1/ | 291-2093 | 2124-4280 |
| adeno-associated virus | GI: 343458969 | (AEM37643.1/ | (AEM37644.1/ |
| 1 isolate I187 Rep78 | | GI: 343458970) | GI: 343458971) |
| and VP1 genes, | | | |
| complete cds. | | | |

Goose parvovirus

| | | | | |
|---|---|---|---|---|
| Goose parvovirus | (U25749.1/ | 1-444 | 537-2420 | 2439-4637 |
| virulent B strain, | GI :1113795) | 4663-5106 | (AAA83229.1/ | (AAA83230.1/ |
| complete genome | | | GI: 1113796) | GI: 1113797) |
| | | | | 2874-4637 |
| | | | | (AAA83231.1/ |
| | | | | GI: 1113798) |
| | | | | 3033-4637 |
| | | | | (AAA83232.1/ |
| | | | | GI: 1113799) |
| Goose parvovirus, | (NC 001701.1/ | 1-444 | 537-2420 | 2439-4637 |
| complete genome | GI: 9628649) | 4663-5106 | (NP_043514.1/ | (NP_043515.1/ |
| | | | GI: 9628650) | GI: 9628651) |
| | | | | 2874-4637 |
| | | | | (NP_043516.1/ |
| | | | | GI: 9628652) |
| | | | | 3033-4637 |
| | | | | (NP_043517.1/ |
| | | | | GI: 9628653) |
| Goose parvovirus strain | (EU583389.1/ | 1-381 | 474-2357 | 2376-4574 |
| 82-0321V, complete | GI: 190888188) | 4600-4980 | (ACE95848.1/ | (ACE95849.1/ |
| genome | | | GI: 190888189) | GI: 190888190) |
| Goose parvovirus strain | (EU583390.1/ | 1-416 | 509-2392 | 2411-4609 |
| 82-0321, complete | GI: 190888191) | 4635-5050 | (ACE95850.1/ | (ACE95851.1/ |
| genome | | | GI: 190888192) | GI: 190888193) |
| Goose parvovirus strain | (EU583391.1/ | 1-418 | 511-2394 | 2413-4611 |
| 06-0329, complete | GI: 190888194) | 4637-5054 | (ACE95852.1/ | ACE95853.1/ |
| genome | | | GI: 190888195) | GI: 190888196) |
| Goose parvovirus strain | (EU583392.1/ | 1-443 | 536-2419 | 2438-4636 |
| VG32/1, complete | GI: 190888197) | 4662-5104 | (ACE95854.1/ | (ACE95855.1/ |
| genome | | | GI: 190888198) | GI: 190888199) |
| Goose parvovirus strain | (JF333590.1/ | 1-444 | 537-2420 | 2439-4637 |
| SH, complete genome | GI: 343113618) | 4663-5106 | (AEL87777.1/ | (AEL87778.1/ |
| | | | GI: 343113619) | GI: 343113620) |
| | | | | 2874-4637 |
| | | | | (AEL87779.1/ |
| | | | | GI: 343113621) |
| | | | | 3033-4637 |
| | | | | (AEL87780.1/ |
| | | | | GI: 343113622) |
| Goose parvovirus strain | (HQ891825.1/ | 1-444 | 537-2420 | 2439-4637 |
| GDaGPV, complete | GI: 359843307) | 4663-5106 | (AEV89789.1/ | (AEV89790.1/ |
| genome | | | GI: 359843308) | GI: 359843309) |
| | | | | 2874-4637 |
| | | | | (AEV89791.1/ |
| | | | | GI: 359843310) |
| | | | | 3033-4637 |
| | | | | (AEV89792.1/ |
| | | | | GI: 359843311) |
| Goose parvovirus strain | (KC478066.1/ | 1-416 | 509-2392 | 2411-4609 |
| SHEX1201, complete | GI: 459360514) | | (AGG56527.1/ | (AGG56528.1/ |
| genome | | | GI: 459360515) | GI: 459360516) |
| Goose parvovirus strain | (KC178571.1/ | 1-444 | 537-2420 | 2439-4637 |
| Y, complete genome | GI: 513129761) | | (AGO17637.1/ | (AGO176381/ |
| | | | GI: 513129762) | GI: 513129763) |
| Goose parvovirus strain | (KC184133.1/ | 1-443 | 536-2419 | 2438-4636 |
| E, complete genome | GI: 513129764) | | (AGO17639.1/ | (AGO17640.1/ |
| | | | GI: 513129765) | GI: 513129766) |
| Goose parvovirus strain | (KC996729.1/ | 1-442 | 535-2418 | 2437-4635 |
| SYG61v, complete | GI: 531997217) | | (AGT62580.1/ | (AGT62581.1/ |
| genome | | | GI: 531997218) | GI: 531997219) |
| Goose parvovirus strain | (KC996730.1/ | 1-414 | 507-2390 | 2409-4607 |
| Y299-6, complete | GI: 531997220) | | (AGT62582.1/ | (AGT62583.1/ |
| genome | | | GI: 531997221) | GI: 531997222) |
| Goose parvovinis | (AF416726.1/ | | 1-1884 | 1903-4101 |
| nonstructural protein | GI: 17226299) | | (AAL37721.1/ | (AAL37722.1/ |
| NS (ns) and capsid | | | GI: 17226300) | GI: 17226301) |
| protein VP (vp) genes, | | | 529-1884 | |
| complete ods. | | | (ABP93843.1/ | |
| | | | GI: 145694447) | |
| Goose parvovirus strain | (EF515837.1/ | | 1-1884 | 1903-4101 |
| DY NS1 (NS1), NS2 | GI: 145694445) | | (ABP93842.1/ | (ABP82770.1/ |
| (NS2), VP1 (VP1), | | | GI: 145694446) | GI: 145573169) |
| VP2 (VP2), and VP3 | | | | 2338-4101 |
| (VP3) genes, complete | | | | (ABP93844.1/ |
| cds. | | | | GI: 145694448) |
| | | | | 2497-4101 |
| | | | | (ABP93845.1/ |
| | | | | GI: 145694449) |
| Goose parvovirus strain | (JF926695.1/ | | 9-1892 | 1911-4109 |
| PT NS1 protein and | GI: 354463162) | | (AER25356.1/ | (AER25357.1/ |
| VPI protein genes, | | | GI: 354463163) | GI: 354463164) |
| complete cds. | | | | |
| Goose parvovirus strain | (JF926696.1/ | | 6-1889 | 1908-4106 |
| D NS1 protein and VP1 | GI: 354463165) | | (AER25358.1/ | (AER25359.1/ |
| protein genes, complete | | | GI: 354463166) | GI: 354463167) |
| cds. | | | | |

Mouse adeno-associated virus 1

| | | | |
|---|---|---|---|
| Mouse adeno- | DQ100362.1/ | 1-327 | 344-2485 |
| associated virus 1 rep | GI: 73665994 | (AAZ79671.1/ | (AAZ79672.1/ |
| gene, partial cds; and | | GI: 73665995) | GI: 73665996) |
| VP1 capsid, VP2 | | | 731-2485 |
| capsid, and VP3 capsid | | | (AAZ79673.1/ |
| genes, complete cds. | | | GI: 73665997) |
| | | | 911-2485 |
| | | | (AAZ79674.1/ |
| | | | GI: 73665998) |

Muscovy duck parvovirus

| | | | | |
|---|---|---|---|---|
| Barbarie duck | U22967.1/ | 1-457 | 548-2431 | 2450-4648 |
| parvovirus REP protein | GI: 1113784 | 4678-5132 | (AAA83224.1/ | (AAA83225.1/ |
| (rep) and three capsid | | | GI: 1113785) | GI: 1113786) |
| protein VP (vp) genes, | | | | 2885-4648 |
| complete cds | | | | (AAA83226.1/ |
| | | | | GI: 1113787) |
| | | | | 3044-4648 |
| | | | | (AAA83227.1/ |
| | | | | GI: 1113788) |
| Muscovy duck | NC_006147.2/ | 1-457 | 548-2431 | 2450-4648 |
| parvovims, complete | GI: 51593841 | 4678-5132 | (YP_068410.1/ | (YP_068411.1/ |
| genome | | | GI: 51593842) | GI: 51593843) |
| | | | | 2885-4648 |
| | | | | (YP_068412.1; |
| | | | | GI: 51593844) |
| | | | | 3044-4648 |
| | | | | (YP_068413.1/ |
| | | | | GI: 51593845) |
| Muscovy duck | JF926697.1/ | | 61-1944 | 1963-4161 |
| parvovims strain P REP | GI: 354463168 | | (AER25360.1/ | (AER25361.1/ |
| protein and VP1 protein | | | GI: 354463169) | GI: 354463170) |
| genes, complete cds. | | | | |
| Muscovy duck | JF926698.1/ | | 61-1944 | 1963-4161 |
| parvovirus strain P1 | GI: 354463171 | | (AER25362.1/ | (AER25363.1 |
| REP protein and VP1 | | | GI: 354463172) | GI: 354463173) |
| protein genes, complete | | | | |
| cds. | | | | |
| Muscovy duck | KC171936.1/ | 1-381 | 512-2395 | 2414-4612 |
| parvovirus isolate | GI: 459256867 | 4681-5061 | (AGG53766.1/ | (AGG53768.1/ |
| SAAS-SHNH, | | | GI: 459256868) | GI: 459256870) |
| complete genome | | | | 2849-4612 |
| | | | | (AGG53769.1/ |
| | | | | GI: 459256871) |
| | | | | 3008-4612 |
| | | | | (AGG53767.1/ |
| | | | | GI: 459256869) |

Serpentine adeno-associated virus 2

| | | | |
|---|---|---|---|
| Serpentine adeno- | EU872429.1/ | 1-642 | 661-1297 |
| associated virus 2 non- | GI: 215401981 | (ACJ66590.1/ | (ACJ66591.1/ |
| structural protein 1 and | | GI: 215401982) | GI: 215401983) |
| capsid protein genes, | | | |
| partial cds | | | |

Snake parvovirus 1

| | | | |
|---|---|---|---|
| Snake parvovirus 1 | AY349010.1/ | 324-2012 | 2030-4210 |
| non-structural protein 1 | GI: 38017148 | (AAR07954.1/ | (AAR07955.1/ |
| (NS1) and capsid | | GI: 38017149) | GI: 38017150) |
| protein (VP1) genes, | | | |
| complete cds | | | |
| Snake parvovirus 1, | NC_006148.1/ | 324-2012 | 2030-4210 |
| complete genome | GI: 51555744 | (YP_068093.1/ | (YP_068094.1/ |
| | | GI: 51555745) | GI: 51555746) |

TABLE 6
Brief Description of the Sequences in the Sequence Listing

| Description | SEQ ID NO: |
|---|---|
| Anti-hTfr CDRs and variable regions | 1-320 |
| | (see Table 1) |
| SpyTag (AA) | 321 |
| Spycatcher (Aa) | 322 |
| KTag (AA) | 323 |
| SpyTag002 (AA) | 324 |
| Isopeptag (AA) | 325 |
| c-myc (AA) | 326 |
| B1 epitope (AA) | 327 |
| 4 linker (AA) | 328 |
| 6 linker (AA) | 329 |
| 8 linker (AA) | 330 |
| 10 linker (AA) | 331 |
| 10 linker (AA) | 332 |
| 12795B scFv (AA) | 333 |
| 12798B scFv (AA) | 334 |
| 12799B scFv (AA) | 335 |
| 12801B scFv (AA) | 336 |
| 12802B scFv (AA) | 337 |
| 12808B scFv (AA) | 338 |
| 12812B scFv (AA) | 339 |
| 12816B scFv (AA) | 340 |
| 12833B scFv (AA) | 341 |
| 12834B scFv (AA) | 342 |
| 12835B scFv (AA) | 343 |
| 12839B scFv (AA) | 344 |
| 12841B scFv (AA) | 345 |
| 12843B scFv (AA) | 346 |
| 12844B scFv (AA) | 347 |
| 12845B scFv (AA) | 348 |
| 12847B scFv (AA) | 349 |
| 12848B scFv (AA) | 350 |
| 12850B scFv (AA) | 351 |
| 31863B scFv (AA) | 352 |
| 31874B scFv (AA) | 353 |
| 69261 scFv (AA) | 354 |
| 69263 scFv (AA) | 355 |
| 69305 scFv (AA) | 356 |
| 69307 scFv (AA) | 357 |
| 69323 scFv (AA) | 358 |
| 69326 scFv (AA) | 359 |
| 69329 scFv (AA) | 360 |
| 69331 scFv (AA) | 361 |
| 69332 scFv (AA) | 362 |
| 69340 scFv (AA) | 363 |
| 69348 scFv (AA) | 364 |
| Anti-hTfr variable regions | 365-388 |
| | (see Table 1) |
| SpyLigase (AA) | 389 |
| SnoopTag (AA) | 390 |
| SnoopCatcher (AA) | 391 |
| pAAV2-CAP wt (DNA) | 392 |
| pAAV2-CAP wt (AA) | 393 |
| pAAV2-CAP R585A R588A (HBM) (DNA) | 394 |
| pAAV2-CAP R585A R588A (HBM) (AA) | 395 |
| pAAV2-CAP N587 SpyTag HBM (DNA) | 396 |
| pAAV2-CAP N587 SpyTag HBM (AA) | 397 |
| pAAV2-CAP N587 Linker 1 SpyTag HBM (DNA) | 398 |
| pAAV2-CAP N587 Linker 1 SpyTag HBM (AA) | 399 |
| pAAV2-CAP N587 Linker 2 SpyTag HBM (DNA) | 400 |
| pAAV2-CAP N587 Linker 2 SpyTag HBM (AA) | 401 |
| pAAV2-CAP N587 Linker 4 SpyTag HBM (DNA) | 402 |
| pAAV2-CAP N587 Linker 4 SpyTag HBM (AA) | 403 |
| pAAV2-CAP N587 Linker 6 SpyTag HBM (DNA) | 404 |
| pAAV2-CAP N587 Linker 6 SpyTag HBM (AA) | 405 |
| pAAV2-CAP N587 Linker 8 SpyTag HBM (DNA) | 406 |
| pAAV2-CAP N587 Linker 8 SpyTag HBM (AA) | 407 |
| pAAV2-CAP N587 Linker 10 SpyTag HBM (DNA) | 408 |
| pAAV2-CAP N587 Linker 10 SpyTag HBM (AA) | 409 |
| pAAV2-CAP G453 SpyTag HBMx5 (DNA) | 410 |
| pAAV2-CAP G453 SpyTag HBMx5 (AA) | 411 |
| pAAV2-CAP G453 Linker10 SpyTag HBMx5 (DNA) | 412 |
| pAAV2-CAP G453 Linker10 SpyTag HBMx5 (AA) | 413 |
| pAAV9-CAP wt (DNA Artificial) | 414 |
| pAAV9-CAP wt (AA) | 415 |
| pAAV9-CAP W503A (DNA) | 416 |
| pAAV9-CAP W503A (AA) | 417 |
| pAAV9-CAP N272A (DNA) | 418 |
| pAAV9-CAP N272A (AA) | 419 |
| pAAV9-CAP A589 SpyTag W503A (DNA) | 420 |
| pAAV9-CAP A589 SpyTag W503A (AA) | 421 |
| pAAV9-CAP A589 Linker10 SpyTag W503A (DNA) | 422 |
| pAAV9-CAP A589 Linker10 SpyTag W503A (AA) | 423 |
| pAAV9-CAP G453 Linker10 SpyTag W503A (DNA) | 424 |
| pAAV9-RC G453 Linker 10 SpyTag W503A (AA) | 425 |
| pAAV8-CAP wt (DNA) | 426 |
| pAAV8-CAP wt (AA) | 427 |
| pAAV1-CAP wt (DNA) | 428 |
| pAAVI-CAP wt (AA) | 429 |
| AAV6 VP1 (AA) | 430 |
| pAAV2-CAP N587 Myc (DNA) | 431 |
| pAAV2-CAP N587 Myc (AA) | 432 |
| IgG-SpyCatcher Linker (AA) | 433 |
| Human transferrin receptor 1 (AA) | 434 |
| Mouse transferrin receptor 1 (AA) | 435 |
| Human transferrin receptor 1, extracellular domain (AA) | 436 |
| Human IgG4 heavy chain-linker - SpyCatcher (DNA) | 437 |
| Human IgG4 heavy chain-linker - SpyCatcher (AA) | 438 |
| Human IgG4 Fab-linker-SpyCatcher (AA) | 439 |
| IgG (DNA) | 440 |
| IgG (AA) | 441 |
| SpyCatcher002 (AA) | 442 |
| bGH pA-specific primer (DNA) | 443 |
| Upstream barcode primer (DNA) | 444 |
| Downstream barcode primer (DNA) | 445 |
| hIgG1 Fc | 446 |

### EXAMPLES

The following examples are provided for illustrative purposes only and are not intended to limit the scope of the invention.

### Results

AAV Particles can be Retargeted to Mouse TJR (mTfR) In Vitro and In Vivo

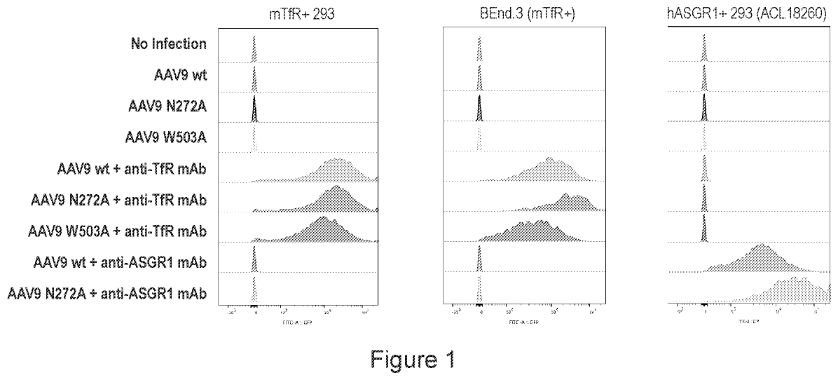

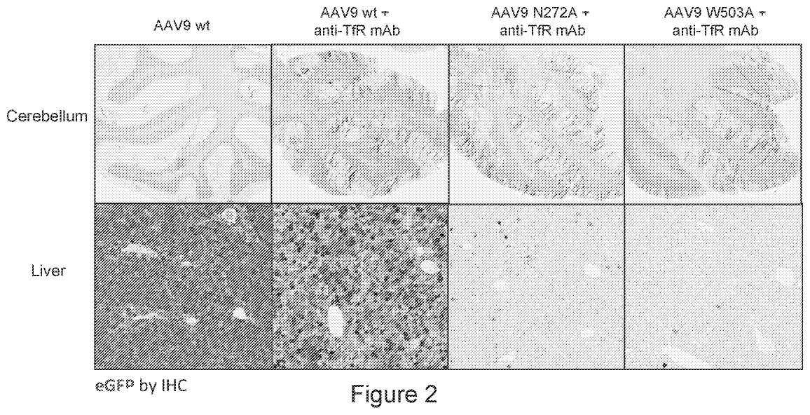

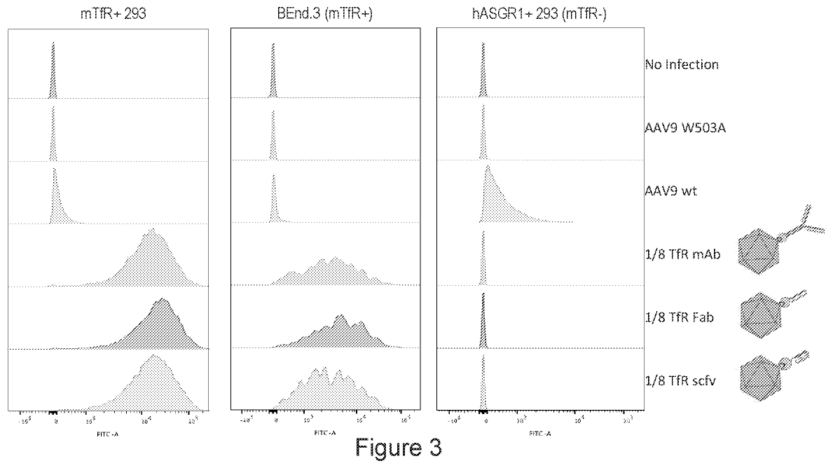

As shown in FIG. 1, AAV particles targeted to mouse TfR via a surrogate anti-mTfR (8D3) antibody specifically infect multiple mTfR+ cell lines in vitro. Moreover, as shown in FIG. 2, AAV9 wildtype particles alone, or conjugated to the anti-mTfR targeting antibody 8D3, transduce the liver of WT C57BL/6J mice in vivo. AAV9 N272A or AAV9 W503A particles conjugated to the anti-mTfR targeting antibody 8D3 are detargeted from the liver and do not facilitate high levels of liver eGFP expression. Compared to mice injected with AAV9 wildtype, brain sections from mice injected with AAV9 wildtype or detargeted particles conjugated to an antibody that binds to mTfR show enhanced eGFP staining in the brain.

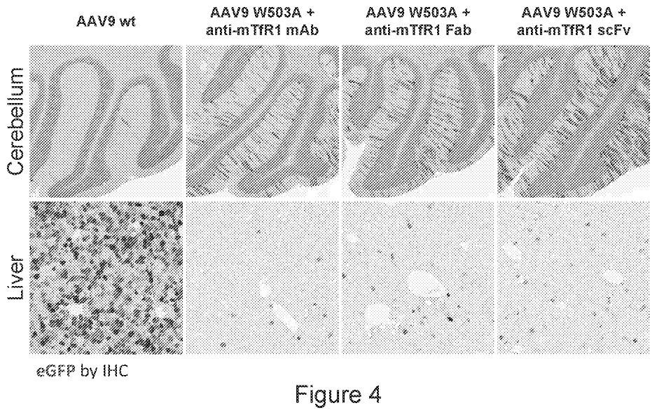

Furthermore, as shown in FIG. 3, mouse surrogate mTfR-binding bivalent mAbs, Fabs, and scFvs conjugated to AAV particles can mediate in vitro transduction in multiple mTfR+ cell lines. And as shown in FIG. 4, AAV9 wildtype particles can transduce the liver of WT C57BL/6J mice, while AAV9 W503A particles are retargeted from the liver and do not facilitate high levels of liver eGFP expression. Compared to mice injected with AAV9 wildtype, brain sections from mice injected with AAV9 W503A particles conjugated to an antibody or variant thereof that binds to mTfR show enhanced eGFP staining in the brain. Increased eGFP staining is observed with AAV9 W503A particles conjugated to an antibody Fab, a scFV, or a bivalent antibody (mAb).

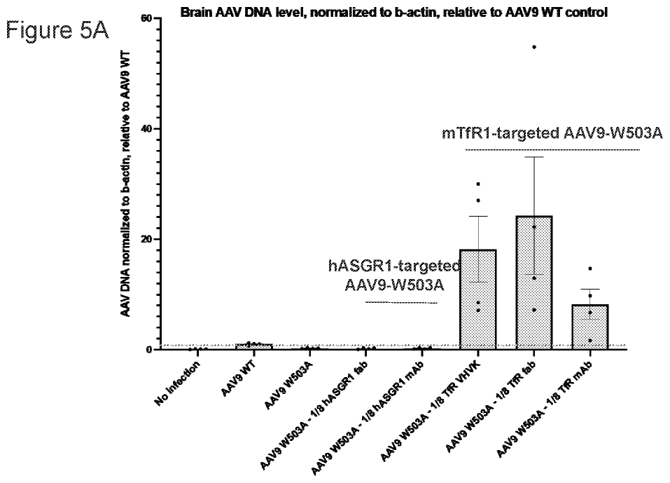

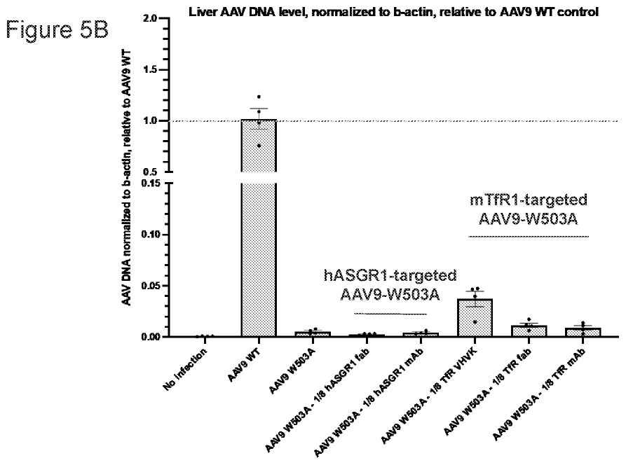

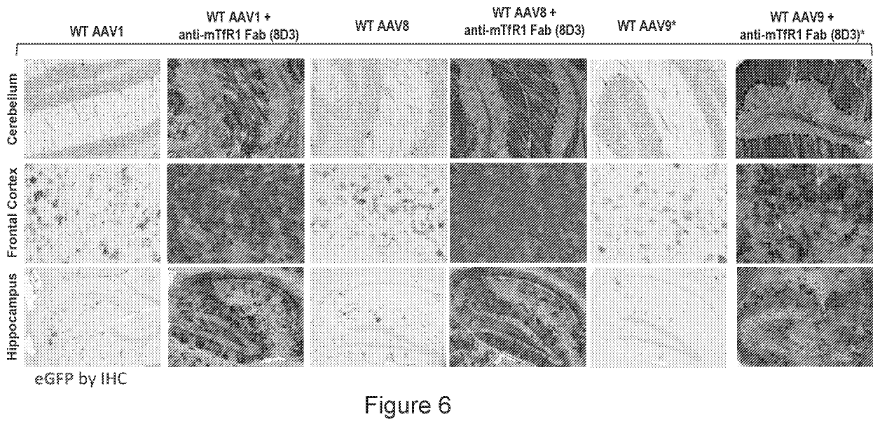

Moreover, as shown in FIG. 5A, AAV DNA from AAV9 W503A particles conjugated to an antibody that binds to mTfR are detected at greater levels in the brain as compared to AAV9 wildtype or AAV9 W503A particles conjugated to an antibody that binds to hASGR1. This is observed in the brain of mice injected with AAV9 W503A particles conjugated to an antibody Fab, a scFV, or a bivalent antibody. In FIG. 5B, AAV9 wildtype DNA is detected at high levels in the liver of WT C57BL/6J mice, while DNA is detected at lower levels from AAV9 W503A particles detargeted from the liver and conjugated to antibodies targeting mTfR, or hASGR1 as a non-targeted control, in variety of antibody formats. In addition, compared to mice injected with wildtype AAV serotypes 1, 8 and 9, brain sections from mice injected with wildtype AAV1, 8 and 9 particles conjugated to a Fab that binds to mTfR show enhanced eGFP staining in the brain (FIG. 6). Accordingly, it was observed that retargeting with TfR improves CNS transduction regardless of serotype used.

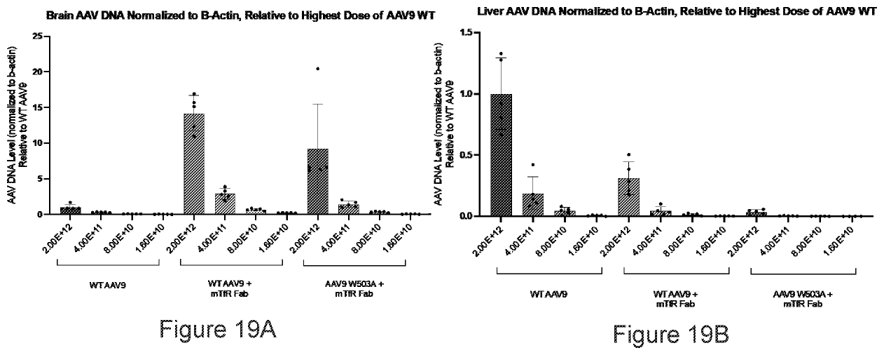

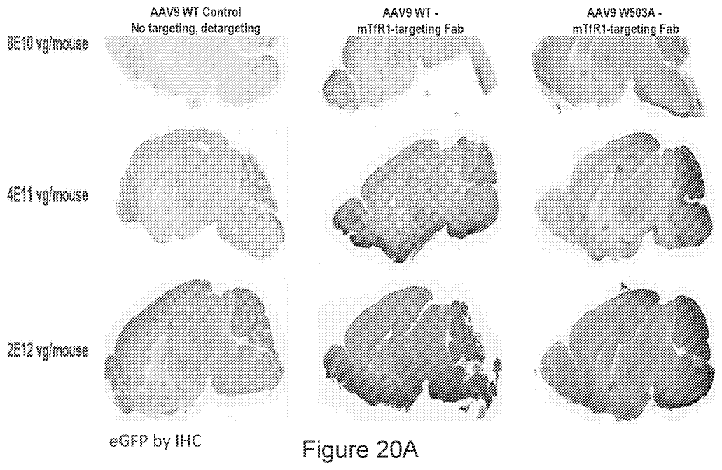

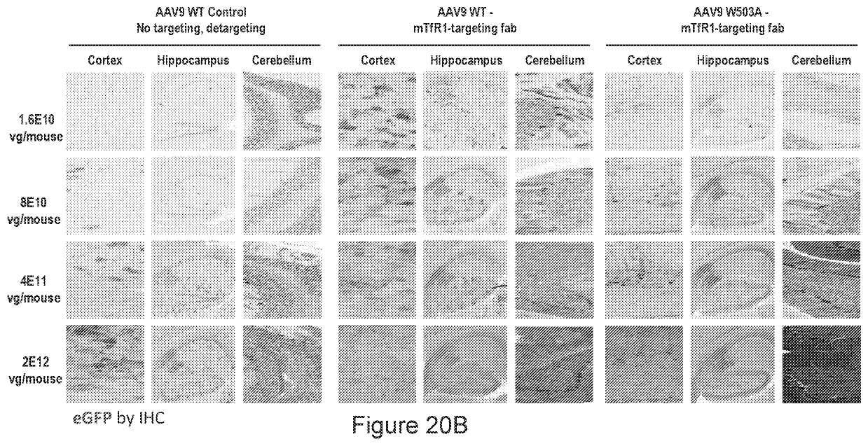

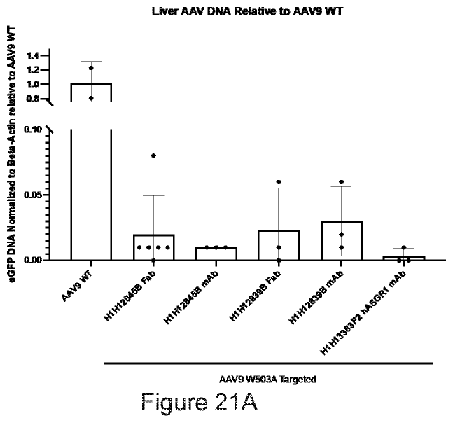

To determine the efficacy of anti-mTfR Fabs conjugated to AAV particles at various doses, a dose escalation study was performed. As shown in FIG. 19A, AAV DNA from WT AAV9 particles conjugated to Fabs that bind to mTfR (“8D3”) or AAV9 W503A particles conjugated to Fabs that bind to mTfR (“8D3”) are detected at greater levels in the brain as compared to WT AAV9. This increase in AAV DNA in the brain with AAV targeted to mTfR is observed at all doses tested. In FIG. 19B, WT AAV9 DNA is detected at high levels in the liver, while DNA from AAV9 W503A particles conjugated to Fabs that bind to mTfR (“8D3”) are detargeted from the liver and are detected at lower levels. This decrease in AAV DNA level in the liver with AAV9 W503A targeted to mTfR is observed at all doses tested. Accordingly, mTfR retargeted AAVs show enhanced delivery of AAV vector DNA to brain, and reduced delivery to liver, relative to WT AAV9 at all doses tested, as measured by qPCR. AAV particles targeted to mTfR demonstrate enhanced brain transduction compared to WT AAV9 in a dose-dependent manner. Moreover, compared to WT AAV9 alone, WT AAV9 or AAV9 W503A retargeted with Fabs to mTfR (“8D3”) results in greater brain transduction at all doses tested (FIG. 20A and FIG. 20B). Accordingly, it is shown that TfR-retargeted WT AAV9 or AAV9 W503A achieves comparable CNS transduction to wild-type (WT) AAV9 at considerably lower doses.

AAV Particles can be Retargeted to Human TfR (hTfR) In Vitro and In Vivo

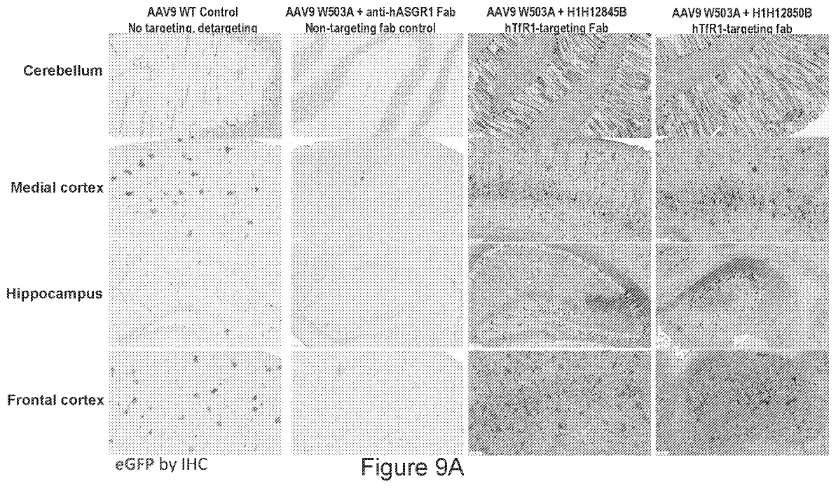

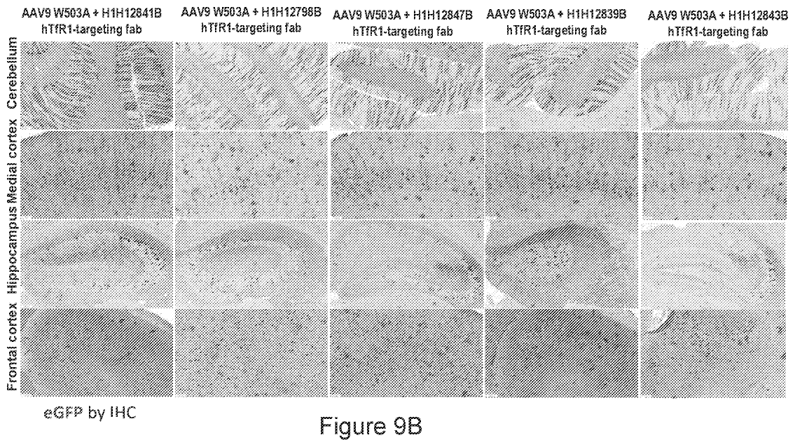

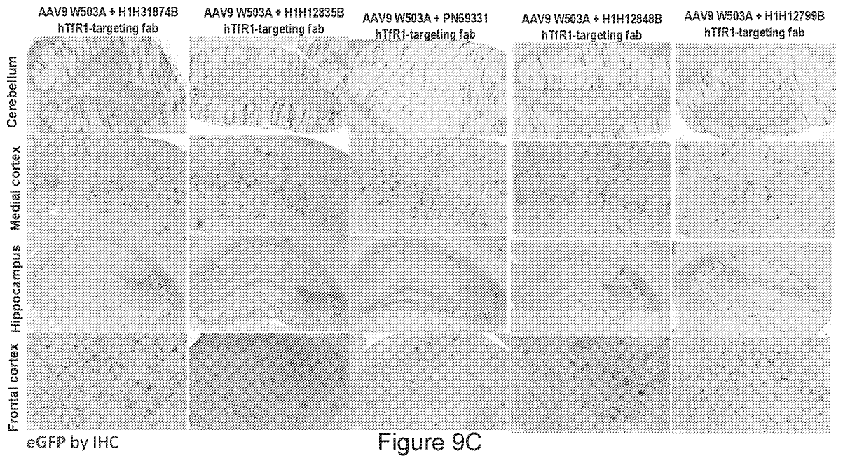

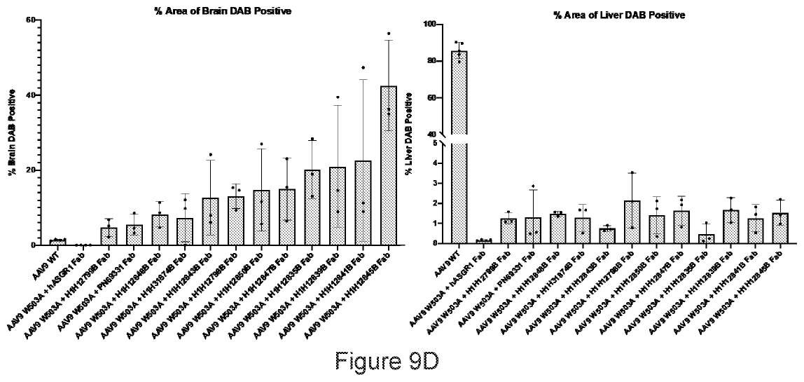

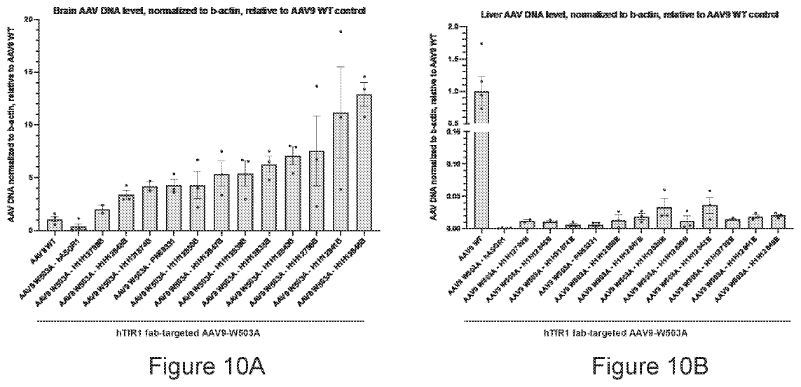

As shown in FIG. 7, AAV particles targeted to human TFR via conjugation to an anti-hTfR Fab specifically infect hTfR+ cell lines in vitro. Moreover, as shown in FIG. 8, AAV9 wildtype particles can transduce the liver of TFRC^{hu/hu} mice, while AAV9 W503A particles are detargeted from the liver and do not facilitate high levels of liver eGFP expression. Compared to wildtype AAV9 and AAV9 W503A particles conjugated to an antibody Fab that binds to hASGR1, AAV9 W503A particles conjugated to antibody Fabs that bind to hTfR show enhanced GFP staining in the brain. In addition, compared to wildtype AAV9 and AAV9 W503A particles conjugated to a Fab that binds to hASGR1, AAV9 W503A particles conjugated to Fabs that bind to hTfR show enhanced GFP staining multiple regions of the brain (FIGS. 9A-9D). Moreover, as shown in FIG. 10A, AAV DNA from AAV9 W503A particles conjugated to Fabs that bind to hTfR are detected at greater levels in the brain as compared to AAV9 WT or AAV9 W503A particles conjugated to a Fab that binds to hASGR1. This increase in AAV DNA is observed with AAV9 W503A particles conjugated to multiple antibody Fabs that bind to hTFR. In FIG. 10B, AAV9 wildtype DNA is detected at high levels in the liver of TFRC^{hu/hu} mice, while DNA from AAV9 W503A particles conjugated to Fabs that bind to hTfR or to hASGR1 are detargeted from the liver and are detected at lower levels. Accordingly, hTfR Fab retargeted AAVs show enhanced delivery of AAV vector DNA to brain, and reduced delivery to liver, as measured by qPCR.

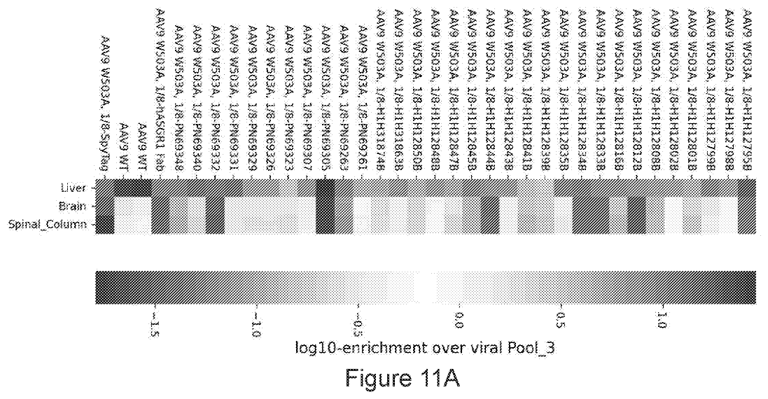

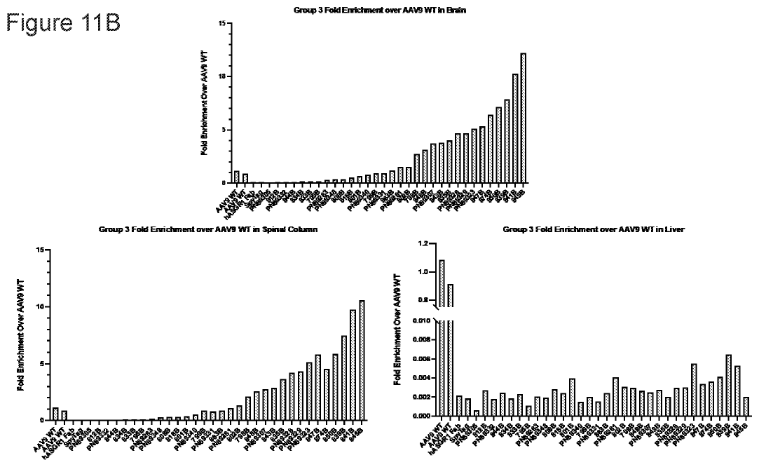

To further demonstrate retargeting of hTfR-conjugated AAV particles, a pool of uniquely barcoded AAV particles was administered to female humanized TFRC mice (TFRC^{hu/hu}). In the liver, wildtype AAV9 alone represents the majority of all barcodes present in the tissue, as expected. In brain and spinal column, detargeted AAV9 (W503A) capsids conjugated to TfR targeting Fabs represent the majority of all barcodes present in the tissues, outperforming AAV9 alone, which accounted for a small percentage of the total barcodes (FIG. 11A and FIG. 11B).

To further understand the tropism of hTfR retargeted AAV particles, various brain cell types in TFRC^{hu/hu} mice were examined. As shown in FIG. 12A and FIG. 12B, AAV particles targeted to human TfR demonstrate transduction across a broad range of multiple brain cell types, including both neuronal and glial cell populations. As shown in FIG. 12A, hTfR fab-retargeted AAVs (WT AAV9 and AAV9 W503A) efficiently transduce multiple neuronal populations, including but not limited to cortical neurons and purkinje cells. In addition, retargeting of both WT AAV9 and AAV9 W503A results in transduction of glial cells, including but not limited to astrocytes and oligodendrocytes. Transduction of some brain microvascular endothelial cells was also observed. As shown in FIG. 12B, hTfR fab-retargeted WT AAV9 robustly transduces neurons and astrocytes, and to a lesser degree, oligodendrocytes. Transduction of these various cell types is observed across a wide range of different brain regions, demonstrating that retargeting with TfR facilitates gene transfer to a variety of cell types in the CNS.

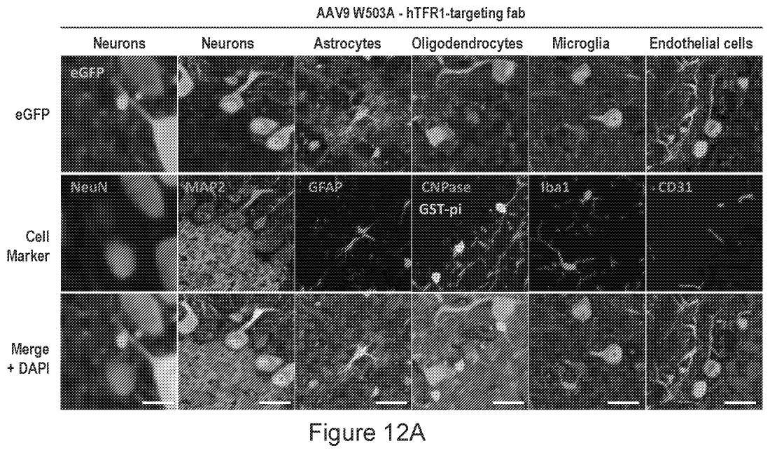

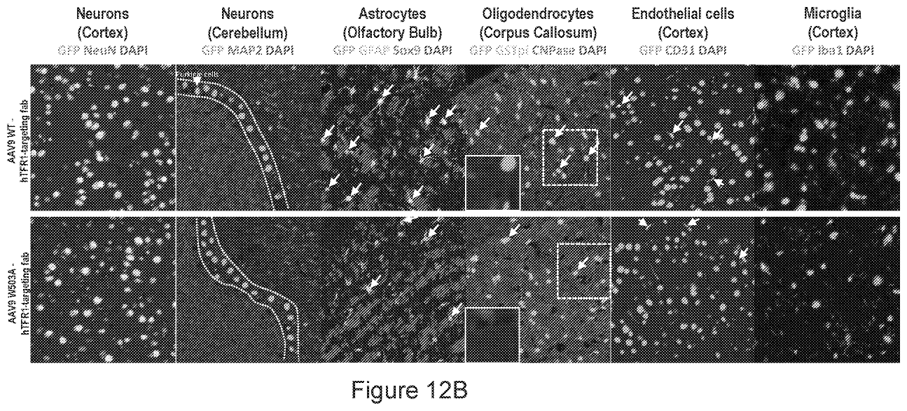

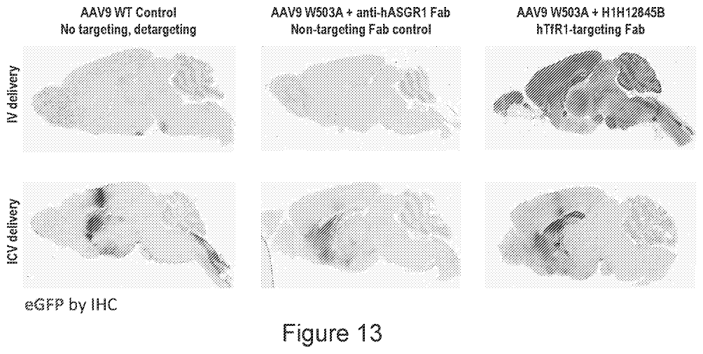

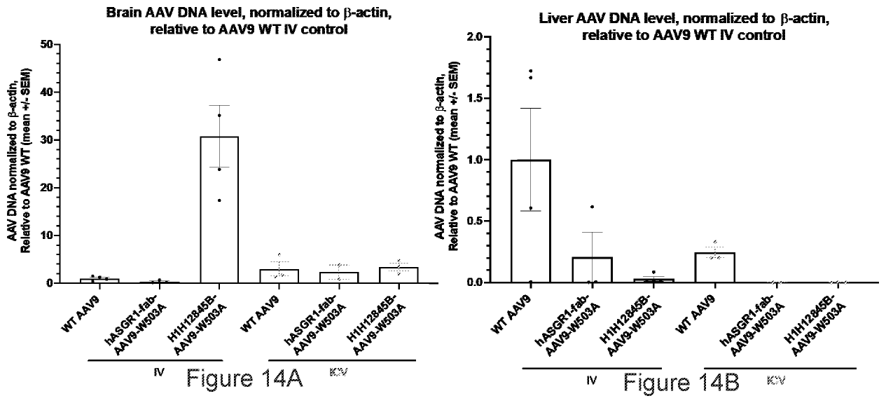

Next, a comparison of intravenous versus intracerebroventricular administration of hTfR retargeted AAV particles was performed. Following intravenous injection, AAV9 W503A particles conjugated to Fabs that bind to hTfR show enhanced eGFP staining in the brain compared to WT AAV9 and AAV9 W503A particles conjugated to a Fab that binds to hASGR1. Following intracerebroventricular injection, eGFP expression in the brain is comparable between targeted or non-targeted AAV. Intravenous injection of AAV results in more widespread brain transduction than intracerebroventricular injection, with AAV9 W503A particles conjugated to Fabs that bind to hTfR showing enhanced transduction across a wide range of brain regions (FIG. 13). As shown in FIG. 14A, following intravenous delivery, AAV DNA from AAV9 W503A particles conjugated to an antibody that binds to hTfR are detected at greater levels in the brain as compared to WT AAV9 or AAV9 W503A particles conjugated to an antibody that binds to hASGR1. Following intracerebroventricular injection, AAV DNA levels in the brain are low and comparable between targeted or non-targeted AAVs. In FIG. 14B, following intravenous delivery, WT AAV9 DNA is detected at high levels in the liver of TFRC^{hu/hu} mice, while DNA is detected at lower levels from AAV9 W503A particles detargeted from the liver and conjugated to Fabs targeting hTfR, or hASGR1 as a non-targeted control. AAV DNA is detected at higher levels in the liver following intravenous injections compared to intracerebroventricular injections of targeted or non-targeted AAVs. AAV particles targeted to human TfR demonstrate enhanced brain transduction after systemic intravenous (IV) delivery compared to intracerebroventricular (ICV) delivery.

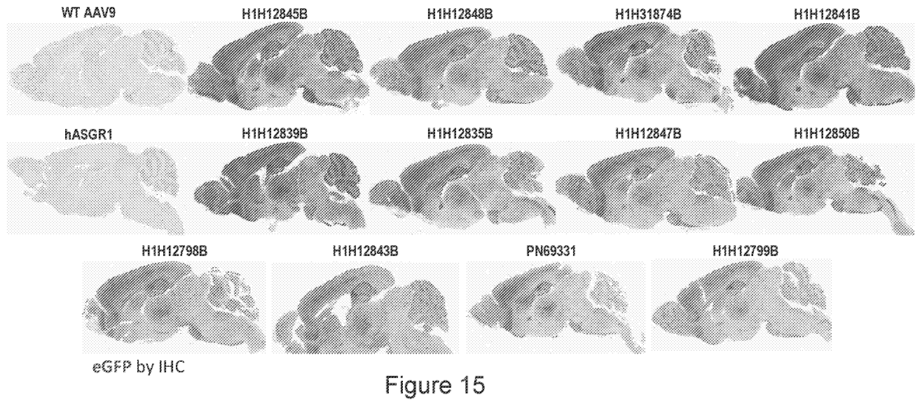

To determine the efficacy for a variety of different antibodies, several different anti-hTfR Fabs were conjugated to AAV particles and administered to TFRC^{hu/hu} mice. Compared to WT AAV9 and AAV9 W503A particles conjugated to a Fab that binds to hASGR1, AAV9 W503A particles conjugated to Fabs that bind to hTfR show enhanced eGFP staining in the brain (FIG. 15).

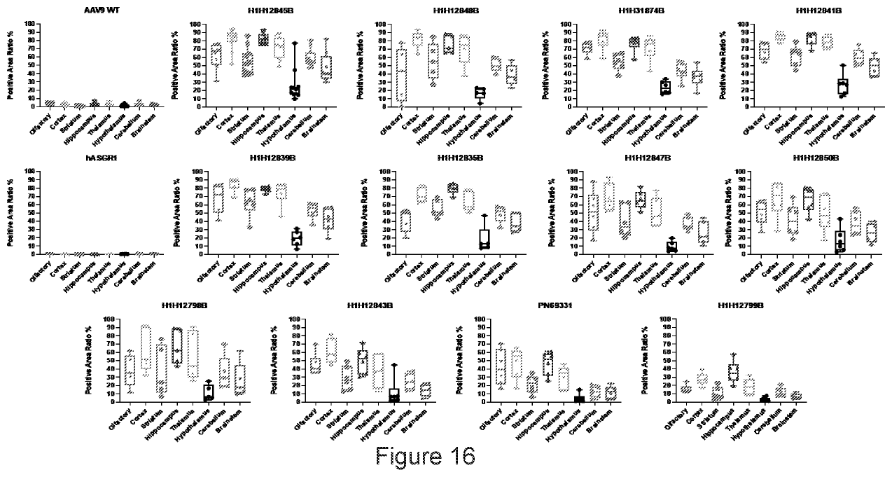

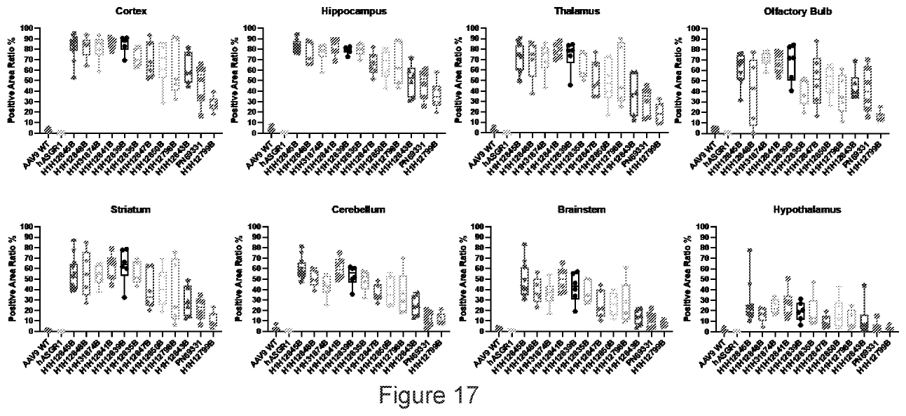

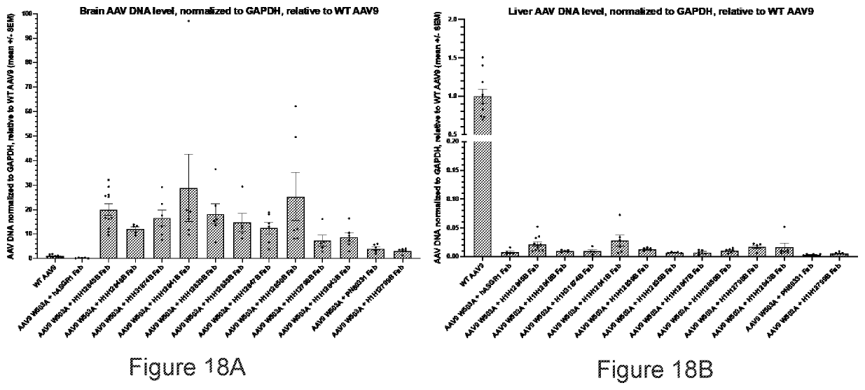

To explore the biodistribution of anti-hTfR Fabs conjugated to AAV particles, eGFP expression in a variety of brain regions in TFRC^{hu/hu} mice was examined. hTfR Fab retargeted AAVs show enhanced delivery to a wide range of brain regions following systemic injection, and enhanced brain transduction relative to WT AAV9 in all brain regions assessed. For AAV9 W503A particles conjugated to Fabs that bind to hTfR, cortex, hippocampus and thalamus show the highest level of eGFP expression, whereas the hypothalamus consistently shows the lowest level of eGFP expression (FIG. 16 and FIG. 17). As an additional confirmation, AAV DNA was measured in the brain (FIG. 18A) and liver (FIG. 18B) of TFRC^{hu/hu} mice administered anti-hTfR Fabs conjugated to AAV particles. As shown in FIG. 18A, AAV DNA from AAV9 W503A particles conjugated to Fabs that bind to hTfR are detected at greater levels in the brain as compared to WT AAV9 or AAV9 W503A particles conjugated to a Fab that binds to hASGR1. This increase in AAV DNA is observed with AAV9 W503A particles conjugated to multiple antibody Fabs that bind to hTFR. In FIG. 18B, WT AAV9 DNA is detected at high levels in the liver of TFRC^{hu/hu} mice, while DNA from AAV9 W503A particles conjugated to Fabs that bind to hTfR or to hASGR1 are detargeted from the liver and are detected at lower levels. Accordingly, hTfR Fab retargeted AAVs show enhanced delivery of AAV vector DNA to brain, and reduced delivery to liver, as measured by qPCR.

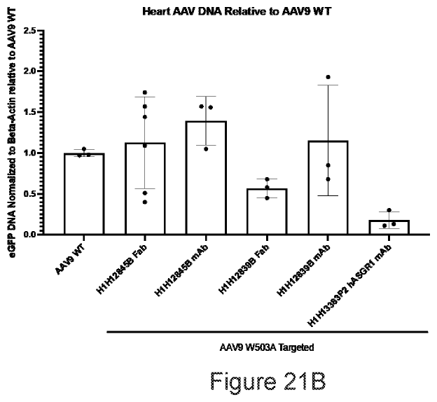

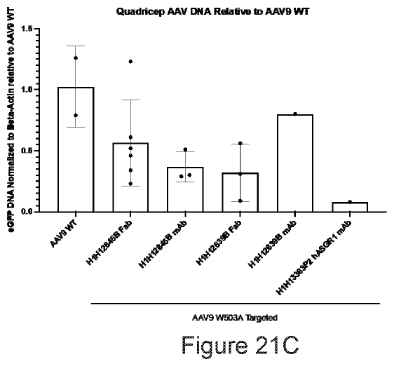

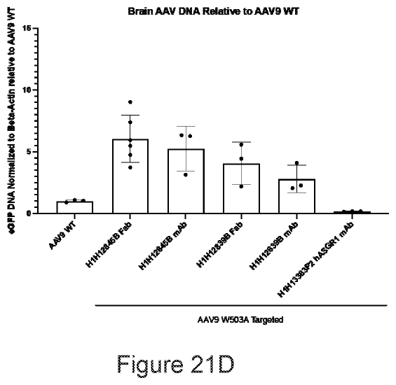

Additionally, hTfR Fab retargeted AAV delivery to various tissues in TFRC^{hu/hu} mice was assessed using qPCR. As shown in FIG. 21A, AAV9 wildtype DNA is detected at high levels in the liver of female TFRC^{hu/hu} mice, while DNA is detected at lower levels from AAV9 W503A particles detargeted from the liver and conjugated to antibodies and Fabs targeting hTfR, or hASGR1 as a non-targeted control. Thus, W503A detargeted, hTfR Fab retargeted AAVs show reduced delivery of AAV vector DNA to liver, as measured by qPCR. In FIG. 21B, AAV9 wildtype DNA is detected at similar levels in the heart of female TFRC^{hu/hu} mice compared to AAV9 W503A particles detargeted from the liver and conjugated to antibodies and Fabs targeting hTfR, and higher than AAV9 W503A particles conjugated to an antibody that binds to hASGR1. Thus, W503A detargeted, hTfR Fab retargeted AAVs show comparable delivery of AAV vector DNA to heart, as measured by qPCR. In FIG. 21C, AAV9 wildtype DNA is detected at similar to higher levels in the quadriceps femoris of female TFRC^{hu/hu} mice compared to AAV9 W503A particles detargeted from the liver and conjugated to antibodies and Fabs targeting hTfR, and higher than AAV9 W503A particles conjugated to an antibody that binds to hASGR1. Thus, W503A detargeted, hTfR Fab retargeted AAVs show comparable delivery of AAV vector DNA to quadriceps femoris, as measured by qPCR. In FIG. 21D, AAV DNA from AAV9 W503A particles conjugated to an antibody or Fab that binds to hTfR are detected at greater levels in the brain as compared to AAV9 wildtype or AAV9 W503A particles conjugated to an antibody that binds to hASGR1. Thus, W503A detargeted, hTfR Fab retargeted AAVs show enhanced delivery of AAV vector DNA to brain, as measured by qPCR.

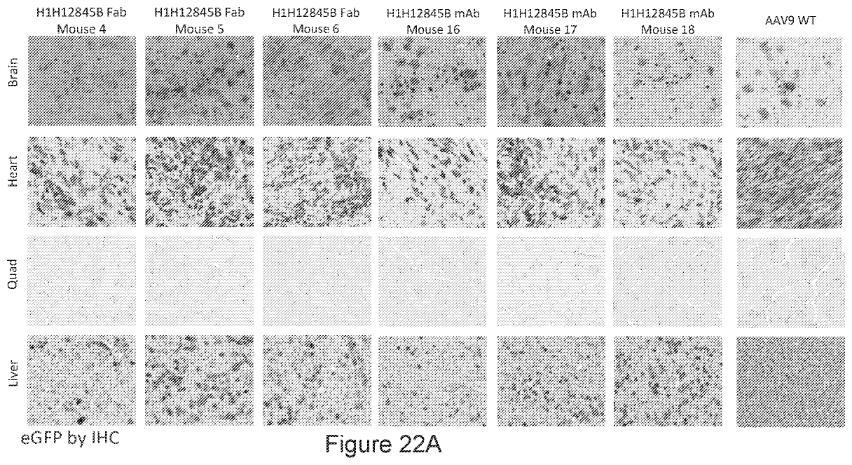

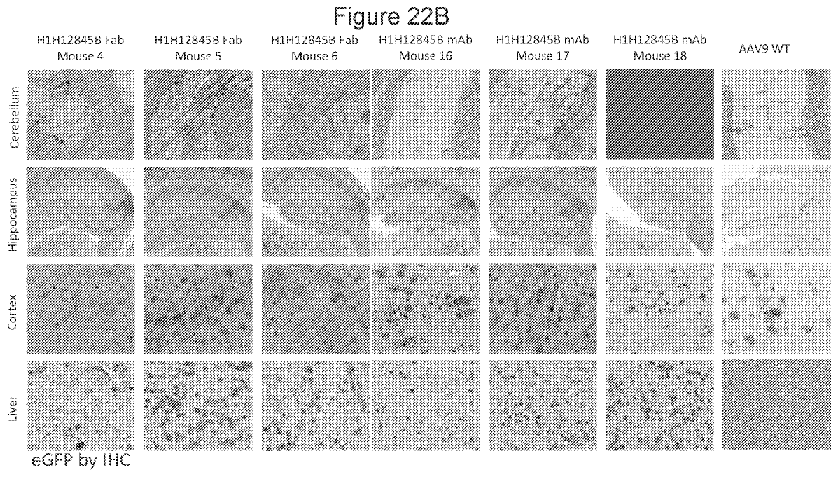

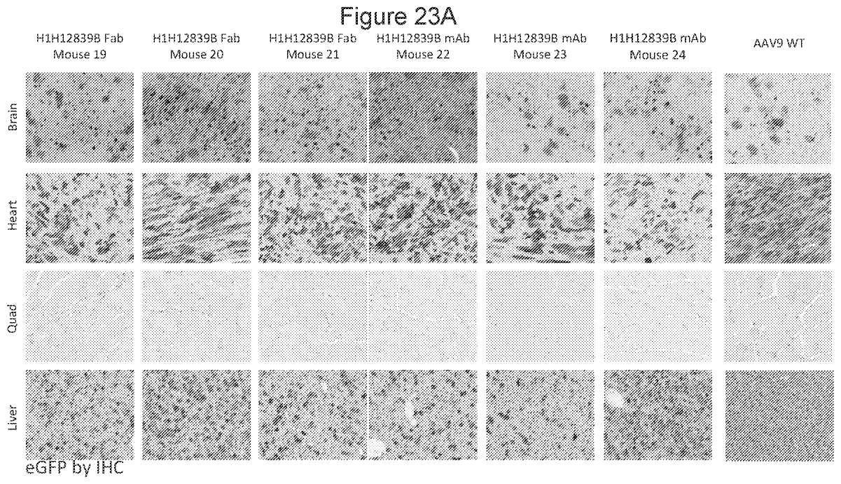

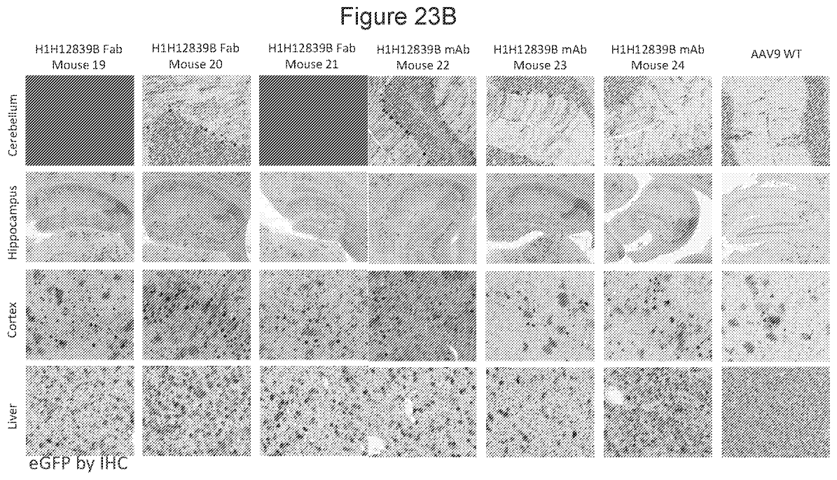

In further experiments, AAV delivery to various tissues in TFRC^{hu/hu} mice using anti-hTfR Fab was compared to delivery using the corresponding anti-hTfR mAb. As shown in FIG. 22A, groups of mice that were injected with detargeted AAV9 W503A conjugated to H1H12845B Fab or H1H12845B mAb show comparable expression of eGFP in brain, heart, quadriceps femoris, and liver. Compared to wildtype AAV9, mice that were injected with detargeted AAV9 W503A conjugated to H1H12845B Fab or H1H12845B mAb showed higher expression of eGFP in the brain, lower expression of eGFP in the heart and liver, and comparable expression of eGFP in the quadriceps femoris. Thus, hTfR Fab and mAb retargeted AAV particles transduce the brain similarly. As shown in FIG. 22B, both groups of mice that were injected with detargeted AAV9 W503A conjugated to H1H12845B Fab or H1H12845B mAb show comparable expression of eGFP in the cerebellum, hippocampus, and cortex, and liver. Compared to wildtype AAV9, mice that were injected with detargeted AAV9 W503A conjugated to H1H12845B Fab or H1H12845B mAb resulted in higher expression of eGFP in the cerebellum, hippocampus, and cortex, and lower expression of eGFP in the liver. Thus, hTfR Fab and mAb retargeted AAV particles transduce the brain similarly. As shown in FIG. 23A, groups of mice that were injected with detargeted AAV9 W503A conjugated to H1H12839B Fab or H1H12839B mAb show comparable expression of eGFP in brain, heart, quadriceps femoris, and liver. Compared to wildtype AAV9, mice that were injected with detargeted AAV9 W503A conjugated to H1H12839B Fab or H1H12839B mAb showed higher expression of eGFP in the brain, lower expression of eGFP in the heart and liver, and comparable expression of eGFP in the quadriceps femoris. Thus, hTfR Fab and mAb retargeted AAV particles transduce the brain similarly. And as shown in FIG. 23B, both groups of mice that were injected with detargeted AAV9 W503A conjugated to H1H12839B Fab or H1H12839B mAb show comparable expression of eGFP in the cerebellum, hippocampus, and cortex, and liver. Compared to wildtype AAV9, mice that were injected with detargeted AAV9 W503A conjugated to H1H12839B Fab or H1H12839B mAb resulted in higher expression of eGFP in the cerebellum, hippocampus, and cortex, and lower expression of eGFP in the liver. Thus, hTfR Fab and mAb retargeted AAV particles transduce the brain similarly.

AAV Particles can be Retargeted to Mouse TfR (mTfR) in Neonates

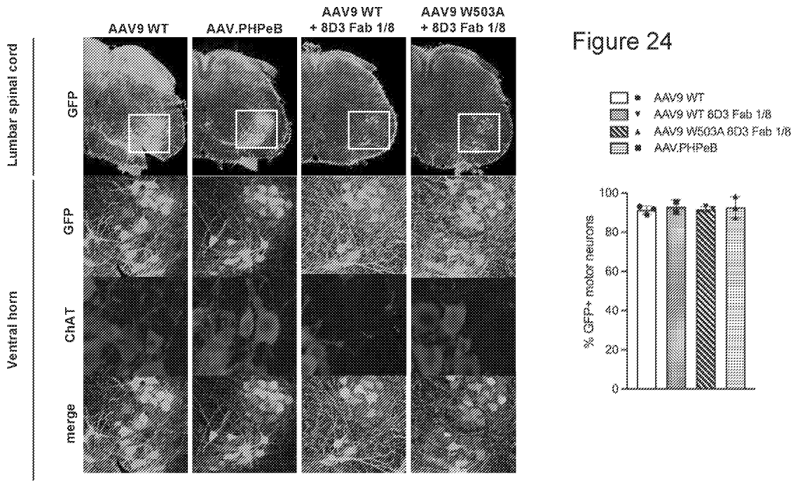

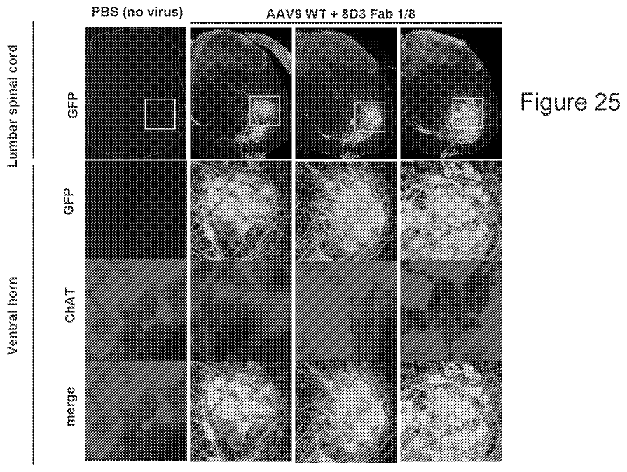

Finally, the delivery of mTfR-retargeted AAVs to neonates was assessed using immunofluorescence imaging. As shown in FIG. 24, and as expected, both WT AAV9 and a modified version (AAV.PHP.eB) have robust spinal cord and motor neuron transduction. Both WT AAV9 and the W503A capsid variant conjugated to TfR antibodies retain the ability to transduce motor neurons, indicating the TfR antibody conjugation does not disrupt the tropism of these viruses for neonatal motor neurons. While untargeted WT AAV9 and AAV.PHP.eB show more intense GFP signal within motor neurons compared to AAV9 targeted to TfR, quantification of the number of motor neurons transduced indicates a transduction efficiency of ~90% for all viruses, indicating retargeted viruses achieve high transduction efficiency despite lower total GFP signal on a per cell basis. Thus, AAV particles targeted to TfR transduce neonatal spinal cord motor neurons when administered directly into the CNS. As shown in FIG. 25, when compared to an intravenously (i.v.) injected PBS negative control mouse, neonatal mice injected via i.v. with retargeted AAV9 display highly robust and reproducible transduction of lumbar spinal cord motor neurons. Thus, AAV particles targeted to TfR highly transduce neonatal spinal cord motor neurons when administered intravenously.

AAV Particles Targeted to TfR can Deliver Functional Cargo to the CNS, Including Nucleic Acid Sequences that Encode Therapeutic Antibodies, and shRNAs for Targeted Gene Knockdown.

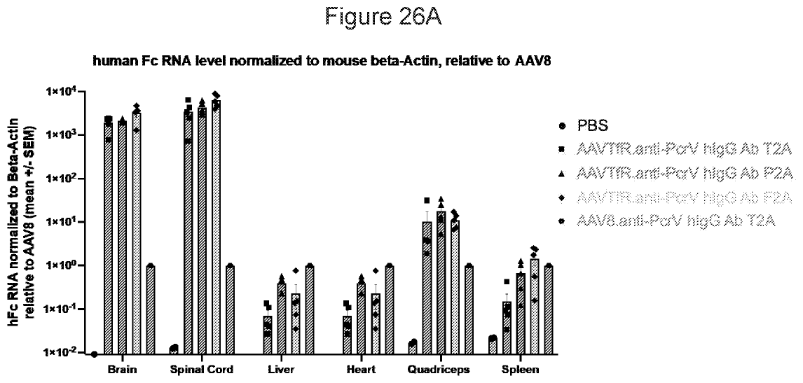

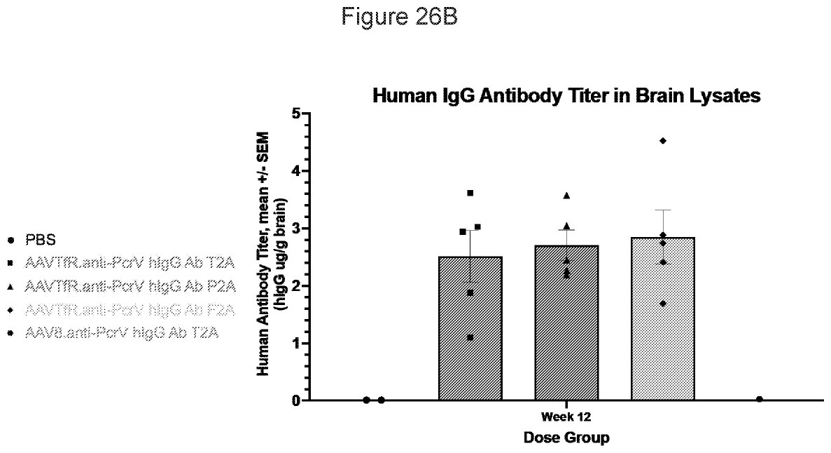

The ability of TfR-retargeted AAVs to mediate expression of a therapeutic payload in the brain via systemic delivery was assessed. To assess the delivery of vectorized antibodies to the brain using mTfR-retargeted AAVs, AAV9 W503A conjugated to scFvs targeting mTfR was compared to AAV8, which is not expected to cross the blood brain barrier and transduce cells in the CNS. TfR-targeted AAV particles were packaged with transgenes that contained a ubiquitously expressing CAGG promoter and gene of interest sequence for a human IgG antibody targeting the Pseudomonas aeruginosa type 3 secretion system (PcrV). For each group tested, the heavy and light chain sequences were separated by a different 2A motif (P2A, T2A, or F2A), which is essential for the creation of steric hindrance and ribosome skipping to allow two polypeptides to be generated from the single AAV-derived mRNA molecule. Robust RNA expression of human IgG antibody sequences was observed in the brain and spinal cord of animals treated with the TfR-targeted AAVs, as shown in FIG. 26A. Thus, AAV targeted to mouse TfR facilitates the expression of secretable antibodies in the CNS after IV delivery. AAV RNA from AAV9 W503A particles conjugated to scFvs that bind to mTfR (“8D3”) was detected at greater levels in the brain and spinal cord as compared to AAV8. This increase in AAV RNA in the CNS with AAV targeted to mTfR is observed with all 2A sequence variant transgenes tested. AAV RNA levels in non-CNS tissues was minimal in animals injected with AAV9 W503A particles conjugated to scFvs that bind to mTfR (“8D3”). Additionally, FIG. 26B shows higher human antibody protein concentrations in the brain lysates of all animals treated with TfR-targeted AAVs than the animals injected with AAV8. Therefore, mTfR-retargeted AAV9 W503A facilitates the delivery and expression of vectorized antibody sequences to the brain via intravenous injection. Thus, AAV targeted to mouse TfR facilitates the expression of secretable antibodies in the CNS after IV delivery.

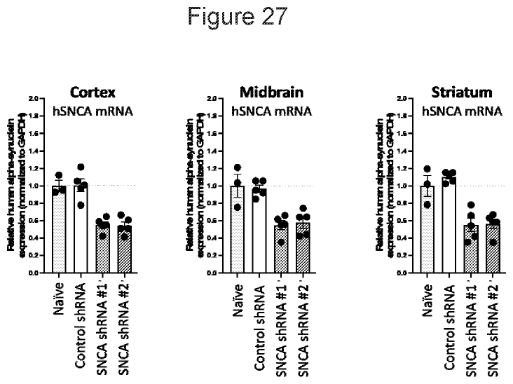

In addition, the ability of TfR1-retargeted AAVs to mediate expression of shRNA and induce the downregulation of a target mRNA in the brain via systemic delivery was assessed. TfR1-targeted AAV particles were packaged with transgenes driving the expression of shRNAs against SNCA. Two SNCA shRNA sequences were tested (SNCA shRNA #1 and SNCA shRNA #2). As shown in FIG. 27, SNCA humanized mice that were injected with detargeted AAV9 W503A conjugated to mTfR1-Fab and expressing either SNCA shRNA #1 or SNCA shRNA #2 showed a 40 to 50% reduction of human SNCA mRNA level in the cortex, midbrain and striatum when compared to naïve SNCA humanized mice and SNCA humanized mice that were injected with detargeted AAV9 W503A conjugated to mTfR1-Fab and expressing a control shRNA. Accordingly, TfR1-AAVs allow the delivery and functional expression of shRNAs and efficiently induce the downregulation of a target mRNA in multiple brain regions. Thus, expression of SNCA shRNAs by AAV targeted to mouse TfR decreases SNCA mRNA levels in the CNS after IV delivery.

### Materials and Methods

### Preparation of AAV Viral Vectors

Virus was generated by transfecting 293T packaging cells using PEI Pro with the following plasmids: pAd Helper, an AAV2 ITR-containing genome plasmid encoding a reporter protein, and a pAAV-CAP plasmid encoding AAV Rep and Cap genes, either with or without additional plasmids encoding either the heavy and light chains of an antibody, Fab or scFv. The antibody or Fab heavy chain constructs or scFvs are all fused to SpyCatcher at their C terminus. Transfection complexes were prepared in incomplete DMEM (no additional supplements) and incubated at room temperature for 10 minutes.

Each virus was generated by transfecting 15 cm plates of 293T packaging cells with the following plasmids and quantities:

| WT AAV9/N272A/W503A GFP | | |
|---|---|---|
| pAd Helper | 16 | ug |
| pscAAV-CBh-eGFP | 8 | ug |
| pAAV9-CAP wt or pAAV9 N272A or pAAV9 W503A | 8 | ug |
| WT AAV1/AAV8 GFP | | |
| pAd Helper | 16 | ug |
| pscAAV-CBh-eGFP | 8 | ug |
| pAAV1-CAP wt or pAAV8 CAP wt | 8 | ug |
| AAV9 wt Anti-Human ASGR1 or anti-mTfR mAb GFP | | |
| pAd Helper | 16 | ug |
| pscAAV-CBh-eGFP | 8 | ug |
| pAAV9 CAP G453 Linker 10 SpyTag W503A | 1 | ug |
| pAAV9-CAP wt | 7 | ug |
| Heavy chain plasmid with C terminal SpyCatcher | 1.5 | ug |
| Light chain plasmid | 3 | ug |
| AAV9 W503A Anti-Human ASGR1 or anti-mTfR mAb GFP | | |
| pAd Helper | 16 | ug |
| pscAAV-CBh-eGFP | 8 | ug |
| pAAV9 CAP G453 Linker 10 SpyTag W503A | 1 | ug |
| pAAV9-CAP W503A | 7 | ug |
| Heavy chain plasmid with C terminal SpyCatcher | 1.5 | ug |
| Light chain plasmid | 3 | ug |
| AAV9 N272A Anti-Human ASGR1 or anti-mTfR mAb GFP | | |
| pAd Helper | 16 | ug |
| pscAAV-CBh-eGFP | 8 | ug |
| pAAV9 CAP G453 Linker 10 SpyTag W503A | 1 | ug |
| pAAV9-CAP N272A | 7 | ug |
| Heavy chain plasmid with C terminal SpyCatcher | 1.5 | ug |
| Light chain plasmid | 3 | ug |
| AAV9 W503A Anti-Human ASGR1 or | | |
| anti-mTfR 8D3 or anti-hTfR Fab GFP | | |
| pAd Helper | 16 | ug |
| pscAAV-CBh-eGFP | 8 | ug |
| pAAV9 CAP G453 Linker 10 SpyTag W503A | 1 | ug |
| pAAV9-CAP W503A | 7 | ug |
| Fab Heavy chain plasmid with C terminal SpyCatcher | 1.5 | ug |
| Light chain plasmid | 3 | ug |
| AAV9 W503A anti-mTfR 8D3 scfv GFP | | |
| pAd Helper | 16 | ug |
| pscAAV-CBh-eGFP | 8 | ug |
| pAAV9 CAP G453 Linker 10 SpyTag W503A | 1 | ug |
| pAAV9-CAP W503A | 7 | ug |
| SpyCatcher VhVk scFv | 4 | ug |
| AAV1 anti-mTfR 8D3 Fab GFP | | |
| pAd Helper | 16 | ug |
| pscAAV-CBh-eGFP | 8 | ug |
| pAAV1 CAP XXX Linker 10 SpyTag | 1 | ug |
| pAAV1-CAP wt | 7 | ug |
| Fab Heavy chain plasmid with C terminal SpyCatcher | 1.5 | ug |
| Light chain plasmid | 3 | ug |
| AAV9 W503A anti-mTfR 8D3 Fab shRNA | | |
| pAd Helper | 16 | ug |
| pAAV-shRNA Expression cassette | 8 | ug |
| pAAV9 CAP G453 Linker 10 SpyTag W503A | 1 | ug |
| pAAV9-CAP W503A | 7 | ug |
| Fab Heavy chain plasmid with C terminal SpyCatcher | 1.5 | ug |
| Light chain plasmid | 3 | ug |
| AAV9 W503A anti-mTfR 8D3 scFv anti-PcrV hIgG | | |
| pAd Helper | 16 | ug |
| pAAV CAGG-anti PcrV hIgG expression cassettes | 8 | ug |
| Each AAV expression cassette utilized a unique 2A motifs: | | |
| P2A, T2A, or F2A | | |
| pAAV9 CAP G453 Linker 10 SpyTag W503A | 1 | ug |
| pAAV9-CAP W503A | 7 | ug |
| SpyCatcher VhVk scFv (8D3) | 4.5 | ug |

Post incubation, complexes are added to DMEM supplemented with 10% FBS, 1×NEAA, 1% Pen/Strep, and 1% L-Glutamine.

Transfected packaging cells were incubated for 3 days at 37° C., then virus was collected from cell lysates using a standard freeze-thaw protocol. In brief, packaging cells were lifted by scraping and pelleted. Supernatant was removed, and cells were resuspended in a solution of 50 mM Tris-HCl; 150 mM NaCl; and 2 mM MgCl2 [pH 8.0]. Intracellular virus particles were released by inducing cell lysis via three consecutive freeze-thaw cycles, consisting of shuttling cell suspension between dry ice/ethanol bath and 37° C. water bath with vigorous vortexing. Viscosity was reduced by treating lysate with EMD Millipore Benzonase (50 U/ml of cell lysate) for 90 min at 37° C., with occasional mixing. Debris was then pelleted by centrifugation, and the resulting supernatant was filtered through a 0.22 μm PVDF Millex-GV Filter. For crude virus to be tested in vitro, crude virus was pipetted into a low-protein-binding tube and stored at 4 C. For virus to be tested in vivo, the clarified lysate is further purified using a four step iodixanol density gradient. Gradients are loaded into a Beckman 70Ti rotor and spun at 66,100 rpm for 1.5 h at 10 C using and max acceleration and deceleration. After ultracentrifugation, iodixanol purified virions are extracted from the 40-60% interface. AAVs in iodixanol solution are diluted in DPBS+/+0.001% pluronic F68 so that the iodixanol is concentration is less than 1%. Purified virus is then concentrated to desired volume using a 100 kDa MWCO Amicon ultrafiltration unit.

Titer (viral genomes per milliliter; vg/mL) was determined by qPCR using a standard curve of a virus of known concentration.

### Cell Lines

All 293 cell lines were maintained in DMEM supplemented with 10% FBS, 1×NEAA, 1% Pen/Strep, and 1% L-glutamine. 293 hASGR1/2 and 293hTfR cell lines were generated by lentiviral transduction of the parental 293 cell line with a vector expressing the corresponding cDNA. All cell lines were obtained from the Regeneron TC core facility.

All 3T3 cell lines were maintained in DMEM supplemented with 10% BCS, 1×NEAA, 1% Pen/Strep, and 1% L-glutamine. 3T3 hTfR cell line was generated by lentiviral transduction of the parental 3T3 cell line with a vector expressing the corresponding cDNA. All cell lines were obtained from the Regeneron TC core facility.

b.End3 cell line was maintained in DMEM supplemented with 10% FBS, 1×NEAA, 1% Pen/Strep, and 1% L-glutamine. Cell line was obtained from the Regeneron TC core facility.

### AAV Capsid Protein Constructs

GeneBlocks encoding the desired SpyTag insertions, flanking linker amino acids, and additional mutations were purchased from IDT and cloned into corresponding digested pAAV-CAP wt plasmids using Gibson Assembly according to the manufacturer's protocol (NEB).

Cell Infection Transduction and Flow Cytometric analysis.

To infect cells, viral particles were added directly to the media of cells in culture, and the mixture was incubated at 37° C. Four days post-infection, cells were trypsinized, resuspended in PBS with 2% FBS, and the percentage of GFP+ cells was collected on a BD FACSCanto flow cytometer and analyzed using FlowJo software.

### Mouse Lines

Humanized TFRC mice (TFRC^{hu/hu}) express human TfR1 and do not express endogenous TfR1.

### In Vivo Analysis of AAV9 GFP Vectors

For intravenous (IV) injections, adult (3-4 month old) WT C57BL/6J mice were tail vein injected with doses of 7.5×10⁹, 1.6×10¹⁰, 5×10¹⁰, 8×10¹⁰, 4×10¹¹ or 2×10¹² vg/mouse of AAV particles. Adult (3-4 month old) female TFRC^{hu/hu} mice were tail vein injected with 1.5×10¹⁰, 1×10¹¹ or 4×10¹¹ vg/mouse of AAV particles. For intracerebroventricular (ICV) injections, adult (3-4 month old) Humanized TFRC (TFRC^{hu/hu}) mice underwent stereotaxic surgeries and received injections with 1×10¹⁰ vg/mouse of AAV particles into the lateral ventricle. Mice were sacrificed 2-3 weeks post-injection, and perfused with saline. Brain and liver were harvested for immunohistochemistry, immunofluorescence and qPCR analysis.

### Barcoding Analysis

Female humanized TFRC mice (TFRC^{hu/hu}) (7-9 week old) were retro-orbitally (RO) injected with 1×10¹¹ vg/mouse or 5×10¹¹ vg/mouse of AAV9 (WT AAV9, AAV PHP.eB, AAV9 W503A anti-ASGR1 Fab, and 32 AAV9 W503A anti-TfR Fabs with barcoded pITR-sc-CBh-eGFP-bGHpA plasmids as the viral genome plasmid). Each of the 36 viruses in the pool was packaged with a version of pITR-sc-CBh-eGFP-bGHpA that carried a unique 32 nucleotide long barcode that was used to quantify transgene expression by that capsid variant. Mice were sacrificed 14 days post injection, and the following organs were harvested for RNA extraction: liver, brain, and spinal cord.

AAV viral vectors were prepared as described above, with one modification: following cell lysis and prior to centrifugation to pellet debris, soluble Spytag peptide (AHIVMVDAYKPTK; SEQ ID NO:321) was added to lysed cell suspensions to a concentration of 100 ug/mL. Lysed cell suspension and soluble Spytag peptide were incubated for 60 min at 37° C. Samples were mixed by equal volume prior to centrifugation.

To analyze barcodes, total RNA isolated from MAID7229 mouse tissues and organs was purified using MagMAX-96 for Microarrays Total RNA Isolation Kit according to manufacturer's specifications. RNA was then treated with ezDNase and cDNA synthesis was performed using SuperScript IV reverse transcriptase and a bGH pA-specific primer (5′-ATCCTCCCCCTTGCTGTCCTGC-3′; SEQ ID NO:443). Barcoded GFP transcripts were amplified from cDNA samples with primers binding upstream (5′-TCGTCGGCAGCGTCAGATGTGTATAAGAGACAGgcactgacaattccgtggtctagg-3′; SEQ ID NO:444) and downstream (5′-GTCTCGTGGGCTCGGAGATGTGTATAAGAGACAGcaaacaacagatggctggcaactag-3′; SEQ ID NO:445) of the barcodes using the Q5 Ultra II 2× master mix. The pooled virus mix was included amongst the samples. Each sample was prepared in three technical replicates for the duration of the library preparation. Amplicons containing the Illumina adapters and unique dual indices (UDI-Illumina) were quantified using qubit and Tapestation, pooled at equimolar ratio, and sequenced on a Nextseq550 using the 300 cycles mid output kit.

### Tropism Studies

Adult (3-4 month old) humanized TFRC mice (TFRC^{hu/hu}) mice were tail vein injected with 1×10¹¹ vg/mouse of AAV particles. Mice were sacrificed 19 days post-injection, perfused with phosphate buffered saline and brains were harvested for histology.

For immunofluorescent staining and evaluation of CNS cell-type tropism, the brain was hemisected in the midsagittal plane and half of the brain was fixed in 10% neutral buffered formalin solution, and then transferred to 70% ethanol 24 hours later. Brains were stored in 70% ethanol for less than a week prior to paraffin embedding. Brains were embedded in paraffin blocks, cut into 4 m-thick sections, and mounted onto TOMO slides. Slides were de-waxed at 60° C. for 1 hour, de-paraffinized by 3×10 minutes washes in Xylene, followed by 3 minutes washes in descending series of ethanol as follows: 100%, 100%, 95%, 95%, 70%, and 50%. Slides were then incubated in 2 minutes incubations as follows: 2×distilled water, 2×TBS. Antigen Retrieval in 1×Tris-EDTA, pH 9 (Abcam #ab93684) was performed using a steamer at 98° C. for 30 minutes. After antigen retrieval, slides were incubated in 0.3% Hydrogen peroxide in TBS to quench endogenous peroxidase activity, washed 5 minutes in TBS and Blocked with TNB Blocking Buffer (0.1M Tris-HCl, 0.15M NaCl, 0.5% Blocking Reagent [Akoya, #NEL700A001], 0.3% Triton X-100) for 30 minutes. The following primary antibodies were used: GFP (Abcam, #13970) at a concentration of 0.1 mg/mL, GST-pi (MBL International, #MBL-312) at 10 μg/mL, CD31 (Abcam, #18281) at 5 μg/mL, MAP2 (Abcam, #ab32454) at 4 μg/mL, Iba1 (Abcam #178847) at 2.4 μg/mL, GFAP (Invitrogen #13-0300) at 2 μg/mL, Sox9 (Abcam #185966) at 5 μg/mL, CNPase (Sigma #MAB326) at 4 μg/mL and NeuN (Abcam, #ab177487) at 3.4 μg/mL. Slides were incubated in primary antibodies overnight at 4° C. Primary antibody was rinsed off with 3×5 minutes washes in TBS and incubated in 0.5×TNB Buffer 1-2 hour with secondary antibodies at concentration of 2 μg/mL and DAPI (Invitrogen, #D3571). The following secondary antibodies were used: Donkey anti-chicken A488 (Invitrogen, #A78948), Donkey anti-rabbit A647 (Invitrogen, #A31573), Donkey anti-rat A594 (Invitrogen, #A21209), Donkey anti-mouse A647 (Invitrogen, #A31574) and Donkey anti-rabbit A594 (Invitrogen, #A32754). Slides were rinsed in several washes of TBS and cover-slipped using anti-fade mounting media (Invitrogen Anti diamond #P36970). Slides were scanned at 20× with Zeiss Axio Scan Z1.

For immunohistochemistry staining followed by brain biodistribution quantifications, the brain was hemisected in the midsagittal plane and half of the brain was fixed in 10% neutral buffered formalin solution, and then transferred to 70% ethanol 24 hours later. Brains were stored in 70% ethanol for less than a week prior to paraffin embedding. Prior to sectioning, the hemibrain was trimmed to include a sagittal plane in each section. Tissues were sectioned using standardized plane sectioning at 4 μm thickness. To obtain representative sagittal brain section images, brain sections underwent standardized CC1 antigen retrieval (56 minutes) and were then treated with an antibody to eGFP (Abcam, #ab13970) at a concentration of 2 μg/mL for 5 hours. Following secondary antibody incubation, eGFP was detected with the Discovery Dab Map detection kit. Immunohistochemistry was performed on the Ventana Discovery Ultra platform. After staining, slides were imaged on an Aperio slide scanner at 40× magnification. For quantitation of GFP expression, 4 μm sagittal sections were taken to match lateral+1.5 mm from midline. Brain sections underwent standardized ER2 antigen retrieval (30 minutes) and were then treated with an antibody to eGFP (Invitrogen, #A11122) at a concentration of 2 μg/mL for 1 hour. Following secondary antibody incubation, eGFP was detected with the Leica Refine detection kit. Immunohistochemistry was performed on the Bond Rx platform. After staining, slides were imaged on an Aperio slide scanner at 40× magnification. The chromogenic IHC images were imported into HALO software to perform annotations and quantitation of the percent area occupied by GFP. Annotations were drawn to allow independent quantitation of several regions throughout the brain. These included: olfactory bulb, cortex, striatum, hippocampus, thalamus, hypothalamus, brain stem and cerebellum. Each set of annotations were modified by hand to match the designated brain area. In HALO analysis algorithm, GFP signal positivity was assessed by DAB optical density thresholding. The above DAB threshold was determined by surpassing background level of DAB staining. The GFP positive area was then normalized to the corresponding annotation area and then plotted as % Positive Area Ratio.

For qPCR, tissues were snap-frozen and stored at −80° C. prior to DNA extraction. Viral DNA was measured in each tissue using a probe recognizing eGFP and was calculated relative to a β-actin or GAPDH housekeeping gene.

### Dose Escalation

Adult (3-4 month old) WT C57BL/6J mice were tail vein injected with 1.6×10¹⁰, 8×10¹⁰, 4×10¹¹, 2×10¹² vg/mouse of AAV particles. Mice were sacrificed 2 weeks post-injection and perfused with phosphate buffered saline. Brain and liver were harvested for immunohistochemistry and qPCR analysis.

For immunohistochemistry staining, the brain was hemisected in the midsagittal plane and half of the brain was fixed in 10% neutral buffered formalin solution, and then transferred to 70% ethanol 24 hours later. Brains were stored in 70% ethanol for less than 1 week prior to paraffin embedding. Prior to sectioning, the hemibrain was trimmed to include a sagittal plane in each section. Tissues were sectioned using standardized plane sectioning at 4 μm thickness. Brain sections underwent standardized ER2 antigen retrieval (30 minutes) and were then treated with an antibody to eGFP (Invitrogen, #A11122) at a concentration of 2 g/mL for 1 hour. Following secondary antibody incubation, eGFP was detected with the Leica Refine detection kit. Immunohistochemistry was performed on the Bond Rx platform. After staining, slides were imaged on an Aperio slide scanner at 40× magnification.

### In Vivo Analysis of AAV Human IgG Vectors

Adult (3-4 month old) WT C57BL/6J mice were retro-orbitally injected with 5e11 vg/mouse of de-targeted AAV9 W503A conjugated to scFv antibodies targeting mTfR (“8D3”) or 5e10 vg/mouse of AAV8. Transgenes contained a ubiquitously expressing CAGG promoter and gene of interest sequence for a human IgG antibody targeting the Pseudomonas aeruginosa type 3 secretion system (PcrV). For each group tested, the heavy and light chain sequences were separated by a different 2A motif (P2A, T2A, or F2A), which is essential for the creation of steric hindrance and ribosome skipping to allow two polypeptides to be generated from the single AAV-derived mRNA molecule. Mice were sacrificed 12 weeks post-injection and perfused with phosphate buffered saline (PBS). Brain, spinal cord, liver, heart, quadriceps, and spleen were harvested for ELISA and qPCR analysis.

For ELISA-based detection of human IgG titer determination in brain, the brain was hemisected in the midsagittal plane and half of the brain was homogenized and lysed in 1×PBS and 2× HALT Protease Phosphotase Inhibitor Cocktail (Thermo Scientific, 78440). ELISA plates were coated with 1 ug/ml AffiniPure Goat Anti-Human IgG, Fcy Fragment (Jackson 109-005-098) at 4° C. overnight then blocked with 3% Bovine Serum Albumin. Diluted mouse brain lysates were applied to the wells at 1 mg total protein/mL and incubated for one hour at room temperature. Goat Anti-Human Kappa, Mouse ads-HRP (Southern Biotech, 2061-05) was used as a secondary antibody. Final detection was performed using SuperSignal ELISA Pico Chemiluminescent Substrate (Thermo, 1856156). Brain lysate sample readouts were interpolated against a purified human IgG standard curve.

In Vivo Analysis of AAV Vectors Expressing SNCA shRNAs

Adult (2-3 month old) SNCA humanized mice were tail vein injected with 4×10¹¹ vg/mouse of de-targeted AAV9 W503A conjugated to Fabs targeting mTfR (“8D3”). Transgenes contained a hU6 promoter driving SNCA shRNA expression and GFP as a fluorescent reporter driven with Ubc promoter. Two SNCA shRNA sequences were tested (SNCA shRNA #1 and SNCA shRNA #2). Mice were sacrificed 1 month post-injection and perfused with phosphate buffered saline (PBS). Brain were harvested, micro dissected, snap-frozen and stored at −80° C. prior to RNA extraction for qPCR analysis. SNCA mRNA level was measured in the cortex, midbrain and striatum using a probe targeting human SNCA mRNA and was calculated relative to a GAPDH housekeeping gene.

### Intracerebroventricular (i.e.v.) Injection in Neonatal Mice

Newborn pups (P0) were anesthetized on ice for ~7-10 minutes until toe pinch reflexes were diminished. Pups were placed under a dissecting microscope with an attached light source and the injection site was cleaned with an alcohol swab. Injections were done using a 10 uL Hamilton syringe (Hamilton cat #7653-01) fitted with a custom removable needle (32 gauge, 12 degree point angle, 0.75 inches long). Pups were given a single injection into the left ventricle at a position approximately 2 mm laterally from the superior sagittal sinus and 2 mm rostrally from the transverse sinus to a depth of approximately 2 mm. Each injection was done with the indicated viral genomes in a volume of 5 uL diluted with PBS and containing FastGreen dye (0.03%) to visualize fluid distribution. After injection, pups were placed on a 37° C. heating pad, monitored for full recovery, and then placed back in their home cages.

### Intravascular (i.v.) Injection (Via Temporal Facial Vein) in Neonatal Mice

Newborn pups (P0) were lightly anesthetized on ice for 5 minutes and then pups were placed on their side (right side up) under a dissecting microscope with an attached light source and the injection site was cleaned with an alcohol swab. Injections were done using a 100 uL Hamilton syringe (Hamilton cat #7638-01) fitted with a removable needle (33 gauge, 12 degree point angle, 0.5 inches long). The facial vein can be easily visualized through the skin in newborn mice up to P2, and was identified spatially as residing immediately anterior to the ear bud and is distinguished from a nearby capillary because it does not move with the skin. Injections were done in a total volume of 40 uL containing PBS and FastGreen dye (0.04%) to visualize fluid distribution. An accurate and optimal injection should lead to dye distribution throughout the entire vasculature within 5 minutes of injection. After injection, pups were placed on a 37° C. heating pad, monitored for full recovery, and then placed back in their home cages.

### Immunofluorescence in Mouse Pup Spinal Cords (Neonatal Injections)

Mice were perfused with ~10 ml of 0.1M phosphate buffer (PB) solution followed by perfusion with ~20 ml 4% paraformaldehyde (PFA) diluted in 0.1M PB. Post-perfusion, brain and spinal cord were kept intact within the skull and vertebral column and post-fixed in 4% PFA for 16-24 hrs at 4° C. followed by removal and replacement with 0.1M PB. Fixed spinal cord was dissected out of the vertebral column and the specific L5 segment was identified by the ventral roots and dissected out. Tissue were embedded in 4% low-melt agarose and sectioned at 70 um using a VT1000 S vibratome (Leica). Sections were blocked overnight at room temperature with 10% normal donkey serum diluted in TBS containing 0.3% Triton-X (TBS-T) and supplemented with 0.05% sodium azide. The next day, sections were incubated with primary antibody diluted in blocking solution for 2 days at room temperature. The antibodies used are as follows: anti-ChAT (Millipore cat #AB144P), 1:200; anti-NeuN (Millipore cat #MAB377), 1:500), and anti-GFP (Invitrogen cat #A10262), 1:4000. Following primary incubation, sections were washed at room temperature six times, 30 minutes each wash with TBS-T, followed by secondary antibody incubation overnight at room temperature diluted in TBS-T. The following secondary antibodies were used: AlexaFluor-647-conjugated donkey anti-goat antibody (Invitrogen, cat #A32849); AlexaFluor-568-conjugated donkey anti-mouse antibody (Invitrogen, cat #A10037); and AlexaFluor-488-conjugated goat anti-chicken antibody (Invitrogen, cat #A32931). After six, 30-minute washes in TBS-T, sections were mounted to slides using Fluoromount-G (SouthernBiotech). Images were acquired using a Zeiss LSM780 confocal microscope. For quantification of virally-transduced (i.e. GFP+) motor neurons, images were acquired using a 20× objective at 3 um steps along the entire z-axis of the section. The total number of GFP+ and GFP− motor neurons was determined by manually counting the ChAT+ cells in the lateral motor column located in the ventral horn. Motor neuron numbers are represented as the average number of motor neurons per section.

Quantitative RT-PCR Analysis of GFP mRNA Expression (Neonatal Injections)

Freshly dissected tissue was immediately placed in RNAlater and stored at −20° C. until RNA isolation. Total RNA was isolated using Trizol reagent followed by DNase treatment. RT-qPCR analysis was performed in a one-step reaction with QuantiNova Probe RT-PCR kit (Qiagen) according to the manufacturer's recommendations. The RT-qPCR contained 2 μL of RNA and an 8 μL of a mixture containing RT-PCR Master mix, ROX dye, RT-mix, and primer-probe mix. The reference assay for Drosha mRNA (cat #Mm01310009_ml) was obtained from ThermoFisher. The GFP mRNA assay (cat #P3-SS1664471-49-52) was obtained from Biosearch Technologies. RT-qPCR reactions were performed in quadruplicate on a ViiA™ 7 Real-Time PCR Detection System (ThermoFisher) with the RT reaction at 45° C. for 10 min followed by PCR cycling (95° C. 10 min, 45 cycles of 95° C. 5 sec, 60° C. 30 s) in an optical 384-well plate. ΔCt values were determined between the target RNA assay (GFP) and the Drosha mRNA reference.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. Although any methods and materials similar or equivalent to those described herein can be used in the practice or testing of the present invention, some preferred methods and materials are now described. All publications cited herein are incorporated herein by reference to describe in their entirety. Unless defined otherwise, all technical and scientific terms used herein have the same meaning as commonly understood by one of ordinary skill in the art to which this invention belongs.

What is claimed is:

1. A recombinant viral capsid protein comprising:
(i) a first member of a protein:protein binding pair inserted and/or displayed by the viral capsid;
(ii) a second member of the protein:protein binding pair, wherein the first member of the protein:protein binding pair and the second member of the protein:protein binding pair are associated; and
(iii) an antibody or binding portion thereof that binds an extracellular domain of a transferrin receptor protein 1 (abbreviated TfR1, TfR or CD71), wherein the antibody or binding portion thereof is fused to the second member of the protein:protein binding pair.

2. The recombinant viral capsid protein of claim 1, wherein the extracellular domain of TfR1 comprises an amino acid sequence set forth in SEQ ID NO:436.

3. The recombinant viral capsid protein of claim 1 or claim 2, wherein the extracellular domain of TfR1 is an extracellular domain of human (h) TfR1.

4. The recombinant viral capsid protein of any one of claims 1-3, further comprising a cell that expresses TfR1 on its surface,
wherein the viral capsid is bound to an extracellular domain of the TfR1 expressed on the surface of the cell.

5. The recombinant viral capsid protein of any one of claim 4, wherein the cell is listed in Table 2.

6. The recombinant viral capsid protein of any one of claims 1-5, further comprising a blood brain barrier endothelial cell that expresses TfR1 on its surface,
wherein the viral capsid is bound to an extracellular domain of the TfR1 expressed on the surface of the blood brain barrier endothelial cell.

7. The recombinant viral capsid protein of any one of claims 1-6, wherein the recombinant viral particle or the composition is in vitro.

8. The recombinant viral capsid protein of any one of claims 1-6, wherein the recombinant viral particle or the composition is in vivo.

9. The recombinant viral capsid protein of any one of claims 1-8, wherein:
(a) the first member of the protein:protein binding pair comprises SpyTag, Isopeptag, SnoopTag, SpyTag002, SpyTag003, or variants thereof,
(b) the second member of the protein:protein binding pair comprises, fused to the targeting ligand, a Spycatcher, KTag, pilin-C, SnoopCatcher, SpyCatcher002, SpyTag003, or variants thereof; and
(c) the first member of the protein:protein binding pair and the second member of the protein:protein binding pair are associated by an isopeptide bond.

10. The recombinant viral capsid protein of any one of claims 1-9, wherein:
(a) the first member of the protein:protein binding pair comprises SpyTag, or a variant thereof, and
(b) the second member of the protein:protein binding pair comprises SpyCatcher, or a variant thereof, fused to the targeting ligand.

11. The recombinant viral capsid protein of any one of claims 1-8, wherein:
(a) the first member of the protein:protein binding pair comprises the c-myc amino acid sequence set forth as SEQ ID NO:326; and
(b) the second member of the protein:protein binding pair comprises a bispecific binding protein comprising an anti-c-myc antibody and the targeting ligand.

12. The recombinant viral capsid protein of any one of claims 1-11, comprising a first and/or second linker operably linking the first member of the protein:protein binding pair to the recombinant viral capsid protein,
wherein each of the first and second linkers is independently at least one amino acid in length.

13. The recombinant viral capsid protein of claim 12, wherein the first and second linker are not identical.

14. The recombinant viral capsid protein of claim 12, wherein the first and second linker are identical.

15. The recombinant viral capsid protein of any one of claims 12-14, wherein the first linker is 10 amino acids in length and/or the second linker is 10 amino acids in length, optionally wherein the amino acid sequence of the first linker and/or the amino acid sequence of the second linker comprises the amino acid sequence set forth as SEQ ID NO:331 or SEQ ID NO:332.

16. The recombinant viral capsid protein of any one of claims 1-15, wherein:
(a) the viral capsid protein comprises an amino acid sequence of a modified VP1 capsid protein, a modified VP2 capsid protein, and/or modified VP3 capsid protein encoded by a mutated cap gene; and
(b) the mutated cap gene or a portion thereof comprises a nucleotide sequence at least 90% identical to a cap gene of an AAV or portion thereof, wherein the mutated cap gene or portion thereof is genetically modified to comprise an insertion of a nucleotide sequence encoding the first member of the protein:protein binding pair such that the modified VP1 capsid protein, the modified VP2 capsid protein and/or the modified VP3 capsid protein comprises the first member of the protein:protein binding pair.

17. The recombinant viral capsid protein of claim 1-16, wherein the mutated cap gene or portion thereof is genetically modified to comprise one or more additional mutations such that the modified VP1 capsid protein, the modified VP2 capsid protein, and/or the modified VP3 capsid protein comprises, in addition to the first member of the protein:protein binding pair:
(i) a point mutation comprising a substitution, insertion, or deletion of an amino acid;
(ii) a chimeric amino acid sequence; or
(iii) both a point mutation and a chimeric amino acid sequence.

18. The recombinant viral capsid protein of claim 17, wherein the substitution, insertion, or deletion of an amino acid reduces the natural tropism of a viral particle comprising the recombinant viral capsid protein and/or creates a detectable label.

19. The recombinant viral capsid protein of any one of claims 1-18, wherein:
(a) the viral capsid protein comprises an amino acid sequence of a modified VP1 capsid protein, a modified VP2 capsid protein, and/or modified VP3 capsid protein encoded by a mutated cap gene; and
(b) the mutated cap gene or a portion thereof comprises a nucleotide sequence at least 90% identical to a cap gene of an AAV or portion thereof, wherein the mutated cap gene or portion thereof is genetically modified to comprise an insertion of a nucleotide sequence encoding the first member of the protein:protein binding pair such that the modified VP1 capsid protein, the modified VP2 capsid protein and/or the modified VP3 capsid protein comprises the first member of the protein:protein binding pair; and
(c) the AAV is selected from the group consisting of AAV1, AAV2, AAV3B, AAV4, AAV5, AAV6, AAV7, AAV8, AAV9, AAV rh10, AAV rh32.33, a non-primate animal AAV listed in Table 5, and a combination thereof.

20. The recombinant viral capsid protein of any one of claims 16-19, wherein the AAV is AAV2.

21. The recombinant viral capsid protein of any one of claims 16-20, wherein the viral capsid protein comprises a modified AAV2 VP1 capsid protein that comprises the first member of the protein:protein binding pair linked, optionally via a linker, to an amino acid at position 1453 and/or 1587.

22. The recombinant viral capsid protein of any one of claims 16-21, wherein the viral capsid protein comprises a modified AAV2 VP1 capsid protein that comprises the first member of the protein:protein binding pair displayed, via a linker, at position G453,
optionally wherein the modified AAV2 VP1 capsid protein further comprises an R585A modification, an R588A modification, or both the R585A modification and the R588A modification, and
optionally wherein the modified AAV2 VP1 capsid protein further comprises an R484A modification, an R487A modification, an R585A modification, an R588A modification, and a K532A modification, or any combination of an R484A modification, an R487A modification, an R585A modification, an R588A modification, and a K532A modification.

23. The recombinant viral capsid protein of any one of claims 16-19, wherein the AAV is AAV9.

24. The recombinant viral capsid protein of any one of claims 16-19 and 23, wherein the viral capsid protein comprises a modified AAV9 VP1 capsid protein that comprises the first member of the protein:protein binding pair linked, optionally via a linker, to an amino acid at position 1453, 1587 or 1589.

25. The recombinant viral capsid protein of any one of claims 16-19 and 23-24, wherein the viral capsid protein comprises a modified AAV9 VP1 capsid protein that comprises the first member of the protein:protein binding pair displayed, via a linker, at G453,
optionally wherein the modified AAV9 VP1 capsid protein further comprises an N272A modification, a W503A modification, or both the N272A modification and the W503A modification.

26. The recombinant viral capsid protein of any one of claims 16-19, wherein the non-primate animal AAV is an avian AAV (AAAV), a non-human mammalian AAV or a squamate AAV.

27. The recombinant viral capsid protein of any one of claims 16-19 and 26, wherein the non-primate animal AAV is an AAAV.

28. The recombinant viral capsid protein of any one of claims 16-19 and 26-27, wherein the viral capsid protein comprises a modified AAAV VP1 capsid protein that comprises the first member of the protein:protein binding pair linked, optionally via a linker, to an amino acid at position 1444 or 1580.

29. The recombinant viral capsid protein of any one of claims 16-19 and 26-28, wherein the viral capsid protein comprises a modified AAAV VP1 capsid protein that comprises the first member of the protein:protein binding pair linked, optionally via a linker, to an amino acid at a position selected from the group consisting of 1429, I430, I431, I432, I433, I434, I436, I437, and I565.

30. The recombinant viral capsid protein of any one of claims 16-19 and 26, wherein the non-primate animal AAV is a squamate AAV.

31. The recombinant viral capsid protein of any one of claims 16-19, 26, and 30, wherein the non-primate animal AAV is a bearded dragon AAV.

32. The recombinant viral capsid protein of any one of claims 16-19, 26 and 30-31, wherein the viral capsid protein comprises a modified bearded dragon VP1 capsid protein that comprises the first member of the protein:protein binding pair linked, optionally via a linker, to an amino acid at position 1573 or 1436.

33. The recombinant viral capsid protein of any one of claims 16-19 and 26, wherein the non-primate animal AAV is a non-human mammalian AAV.

34. The recombinant viral capsid protein of any one of claims 16-19, 26, and 33, wherein the non-primate animal AAV is a sea lion AAV.

35. The recombinant viral capsid protein of any one of claims 1-34, wherein the antibody or binding portion thereof that binds an extracellular domain of TfR1 binds the same epitope on the extracellular domain of TfR1 as a reference antibody comprising an HCVR/LCVR amino acid sequence pair as set forth in Table 1.

36. The recombinant viral capsid protein of any one of claims 1-35, wherein the antibody or binding portion thereof that binds an extracellular domain of TfR1 comprises heavy chain complementarity determining regions (HCDR1, HCDR2 and HCDR3) from a heavy chain variable region (HCVR) comprising an amino acid sequence set forth in SEQ ID NO: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302 or 312 (or a variant thereof); and/or
light chain complementarity determining regions (LCDR1, LCDR2 and LCDR3) from a light chain variable region (LCVR) comprising an amino acid sequence set forth in SEQ ID NO: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307 or 317 (or a variant thereof).

37. The recombinant viral capsid protein of any one of claims 1-36, wherein the antibody or binding portion thereof that binds an extracellular domain of TfR1 comprises:
(i) a HCVR comprising the HCDR1, HCDR2 and HCDR3 of a HCRV that comprises the amino acid sequence set forth in SEQ ID NO: 2 (or a variant thereof); and a LCVR comprising the LCDR1, LCDR2 and LCDR3 of a LCRV that comprises the amino acid sequence set forth in SEQ ID NO: 7 (or a variant thereof);
(ii) a HCVR comprising the HCDR1, HCDR2 and HCDR3 of a HCRV that comprises the amino acid sequence set forth in SEQ ID NO: 12 (or a variant thereof); and a LCVR comprising the LCDR1, LCDR2 and LCDR3 of a LCRV that comprises the amino acid sequence set forth in SEQ ID NO: 17 (or a variant thereof);
(iii) a HCVR comprising the HCDR1, HCDR2 and HCDR3 of a HCRV that comprises the amino acid sequence set forth in SEQ ID NO: 22 (or a variant thereof); and a LCVR comprising the LCDR1, LCDR2 and LCDR3 of a LCRV that comprises the amino acid sequence set forth in SEQ ID NO: 27 (or a variant thereof);
(iv) a HCVR comprising the HCDR1, HCDR2 and HCDR3 of a HCRV that comprises the amino acid sequence set forth in SEQ ID NO: 32 (or a variant thereof); and a LCVR comprising the LCDR1, LCDR2 and LCDR3 of a LCRV that comprises the amino acid sequence set forth in SEQ ID NO: 37 (or a variant thereof);
(v) a HCVR comprising the HCDR1, HCDR2 and HCDR3 of a HCRV that comprises the amino acid sequence set forth in SEQ ID NO: 42 (or a variant thereof); and a LCVR comprising the LCDR1, LCDR2 and LCDR3 of a LCRV that comprises the amino acid sequence set forth in SEQ ID NO: 47 (or a variant thereof);
(vi) a HCVR comprising the HCDR1, HCDR2 and HCDR3 of a HCRV that comprises the amino acid sequence set forth in SEQ ID NO: 52 (or a variant thereof); and a LCVR comprising the LCDR1, LCDR2 and LCDR3 of a LCRV that comprises the amino acid sequence set forth in SEQ ID NO: 57 (or a variant thereof);
(vii) a HCVR comprising the HCDR1, HCDR2 and HCDR3 of a HCRV that comprises the amino acid sequence set forth in SEQ ID NO: 62 (or a variant thereof); and a LCVR comprising the LCDR1, LCDR2 and LCDR3 of a LCRV that comprises the amino acid sequence set forth in SEQ ID NO: 67 (or a variant thereof);
(viii) a HCVR comprising the HCDR1, HCDR2 and HCDR3 of a HCRV that comprises the amino acid sequence set forth in SEQ ID NO: 72 (or a variant thereof); and a LCVR comprising the LCDR1, LCDR2 and LCDR3 of a LCRV that comprises the amino acid sequence set forth in SEQ ID NO: 77 (or a variant thereof);
(ix) a HCVR comprising the HCDR1, HCDR2 and HCDR3 of a HCRV that comprises the amino acid sequence set forth in SEQ ID NO: 82 (or a variant thereof); and a LCVR comprising the LCDR1, LCDR2 and LCDR3 of a LCRV that comprises the amino acid sequence set forth in SEQ ID NO: 87 (or a variant thereof);
(x) a HCVR comprising the HCDR1, HCDR2 and HCDR3 of a HCRV that comprises the amino acid sequence set forth in SEQ ID NO: 92 (or a variant thereof); and a LCVR comprising the LCDR1, LCDR2 and LCDR3 of a LCRV that comprises the amino acid sequence set forth in SEQ ID NO: 97 (or a variant thereof);
(xi) a HCVR comprising the HCDR1, HCDR2 and HCDR3 of a HCRV that comprises the amino acid sequence set forth in SEQ ID NO: 102 (or a variant thereof); and a LCVR comprising the LCDR1, LCDR2 and LCDR3 of a LCRV that comprises the amino acid sequence set forth in SEQ ID NO: 107 (or a variant thereof);
(xii) a HCVR comprising the HCDR1, HCDR2 and HCDR3 of a HCRV that comprises the amino acid sequence set forth in SEQ ID NO: 112 (or a variant thereof); and a LCVR comprising the LCDR1, LCDR2 and LCDR3 of a LCRV that comprises the amino acid sequence set forth in SEQ ID NO: 117 (or a variant thereof);
(xiii) a HCVR comprising the HCDR1, HCDR2 and HCDR3 of a HCRV that comprises the amino acid sequence set forth in SEQ ID NO: 122 (or a variant thereof); and a LCVR comprising the LCDR1, LCDR2 and LCDR3 of a LCRV that comprises the amino acid sequence set forth in SEQ ID NO: 127 (or a variant thereof);
(xiv) a HCVR comprising the HCDR1, HCDR2 and HCDR3 of a HCRV that comprises the amino acid sequence set forth in SEQ ID NO: 132 (or a variant thereof); and a LCVR comprising the LCDR1, LCDR2 and LCDR3 of a LCRV that comprises the amino acid sequence set forth in SEQ ID NO: 137 (or a variant thereof);
(xv) a HCVR comprising the HCDR1, HCDR2 and HCDR3 of a HCRV that comprises the amino acid sequence set forth in SEQ ID NO: 142 (or a variant thereof); and a LCVR comprising the LCDR1, LCDR2 and LCDR3 of a LCRV that comprises the amino acid sequence set forth in SEQ ID NO: 147 (or a variant thereof);
(xvi) a HCVR comprising the HCDR1, HCDR2 and HCDR3 of a HCRV that comprises the amino acid sequence set forth in SEQ ID NO: 152 (or a variant thereof); and a LCVR comprising the LCDR1, LCDR2 and LCDR3 of a LCRV that comprises the amino acid sequence set forth in SEQ ID NO: 157 (or a variant thereof);
(xvii) a HCVR comprising the HCDR1, HCDR2 and HCDR3 of a HCRV that comprises the amino acid sequence set forth in SEQ ID NO: 162 (or a variant thereof); and a LCVR comprising the LCDR1, LCDR2 and LCDR3 of a LCRV that comprises the amino acid sequence set forth in SEQ ID NO: 167 (or a variant thereof);
(xviii) a HCVR comprising the HCDR1, HCDR2 and HCDR3 of a HCRV that comprises the amino acid sequence set forth in SEQ ID NO: 172 (or a variant thereof); and a LCVR comprising the LCDR1, LCDR2 and LCDR3 of a LCRV that comprises the amino acid sequence set forth in SEQ ID NO: 177 (or a variant thereof);
(xix) a HCVR comprising the HCDR1, HCDR2 and HCDR3 of a HCRV that comprises the amino acid sequence set forth in SEQ ID NO: 182 (or a variant thereof); and a LCVR comprising the LCDR1, LCDR2 and LCDR3 of a LCRV that comprises the amino acid sequence set forth in SEQ ID NO: 187 (or a variant thereof);
(xx) a HCVR comprising the HCDR1, HCDR2 and HCDR3 of a HCRV that comprises the amino acid sequence set forth in SEQ ID NO: 192 (or a variant thereof); and a LCVR comprising the LCDR1, LCDR2 and LCDR3 of a LCRV that comprises the amino acid sequence set forth in SEQ ID NO: 197 (or a variant thereof);
(xxi) a HCVR comprising the HCDR1, HCDR2 and HCDR3 of a HCRV that comprises the amino acid sequence set forth in SEQ ID NO: 202 (or a variant thereof); and a LCVR comprising the LCDR1, LCDR2 and LCDR3 of a LCRV that comprises the amino acid sequence set forth in SEQ ID NO: 207 (or a variant thereof);
(xxii) a HCVR comprising the HCDR1, HCDR2 and HCDR3 of a HCRV that comprises the amino acid sequence set forth in SEQ ID NO: 212 (or a variant thereof); and a LCVR comprising the LCDR1, LCDR2 and LCDR3 of a LCRV that comprises the amino acid sequence set forth in SEQ ID NO: 217 (or a variant thereof);
(xxiii) a HCVR comprising the HCDR1, HCDR2 and HCDR3 of a HCRV that comprises the amino acid sequence set forth in SEQ ID NO: 222 (or a variant thereof); and a LCVR comprising the LCDR1, LCDR2 and LCDR3 of a LCRV that comprises the amino acid sequence set forth in SEQ ID NO: 227 (or a variant thereof);
(xiv) a HCVR comprising the HCDR1, HCDR2 and HCDR3 of a HCRV that comprises the amino acid sequence set forth in SEQ ID NO: 232 (or a variant thereof); and a LCVR comprising the LCDR1, LCDR2 and LCDR3 of a LCRV that comprises the amino acid sequence set forth in SEQ ID NO: 237 (or a variant thereof);
(xv) a HCVR comprising the HCDR1, HCDR2 and HCDR3 of a HCRV that comprises the amino acid sequence set forth in SEQ ID NO: 242 (or a variant thereof); and a LCVR comprising the LCDR1, LCDR2 and LCDR3 of a LCRV that comprises the amino acid sequence set forth in SEQ ID NO: 247 (or a variant thereof);
(xvi) a HCVR comprising the HCDR1, HCDR2 and HCDR3 of a HCRV that comprises the amino acid sequence set forth in SEQ ID NO: 252 (or a variant thereof); and a LCVR comprising the LCDR1, LCDR2 and LCDR3 of a LCRV that comprises the amino acid sequence set forth in SEQ ID NO: 257 (or a variant thereof);
(xvii) a HCVR comprising the HCDR1, HCDR2 and HCDR3 of a HCRV that comprises the amino acid sequence set forth in SEQ ID NO: 262 (or a variant thereof); and a LCVR comprising the LCDR1, LCDR2 and LCDR3 of a LCRV that comprises the amino acid sequence set forth in SEQ ID NO: 267 (or a variant thereof);
(xviii) a HCVR comprising the HCDR1, HCDR2 and HCDR3 of a HCRV that comprises the amino acid sequence set forth in SEQ ID NO: 272 (or a variant thereof); and a LCVR comprising the LCDR1, LCDR2 and LCDR3 of a LCRV that comprises the amino acid sequence set forth in SEQ ID NO: 277 (or a variant thereof);
(xix) a HCVR comprising the HCDR1, HCDR2 and HCDR3 of a HCRV that comprises the amino acid sequence set forth in SEQ ID NO: 282 (or a variant thereof); and a LCVR comprising the LCDR1, LCDR2 and LCDR3 of a LCRV that comprises the amino acid sequence set forth in SEQ ID NO: 287 (or a variant thereof);
(xxx) a HCVR comprising the HCDR1, HCDR2 and HCDR3 of a HCRV that comprises the amino acid sequence set forth in SEQ ID NO: 292 (or a variant thereof); and a LCVR comprising the LCDR1, LCDR2 and LCDR3 of a LCRV that comprises the amino acid sequence set forth in SEQ ID NO: 297 (or a variant thereof);
(xxxi) a HCVR comprising the HCDR1, HCDR2 and HCDR3 of a HCRV that comprises the amino acid sequence set forth in SEQ ID NO: 302 (or a variant thereof); and a LCVR comprising the LCDR1, LCDR2 and LCDR3 of a LCRV that comprises the amino acid sequence set forth in SEQ ID NO: 307 (or a variant thereof); and/or
(xxxii) a HCVR comprising the HCDR1, HCDR2 and HCDR3 of a HCRV that comprises the amino acid sequence set forth in SEQ ID NO: 312 (or a variant thereof); and a LCVR comprising the LCDR1, LCDR2 and LCDR3 of a LCRV that comprises the amino acid sequence set forth in SEQ ID NO: 317 (or a variant thereof).

38. The recombinant viral capsid protein of any one of claims 1-37, wherein the antibody or binding portion thereof that binds an extracellular domain of TfR1 comprises:
(a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and
a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof);
(b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and
a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof);
(c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and
a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof);
(d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and
a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof);
(e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and
a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof);
(f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and
a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof);
(g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and
a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof);
(h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and
a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof);
(i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and
a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof);
(j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and
a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof);
(k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and
a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof);
(l) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and
a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof);
(m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and
a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof);
(n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and
a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof);
(o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and
a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof);
(p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and
a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof);
(q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and
a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof);
(r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and
a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof);
(s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and
a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof);
(t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and
a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof);
(u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and
a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof);
(v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and
a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof);
(w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and
a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof);
(x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and
a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof);
(y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and
a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof);
(z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and
a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof);
(aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and
a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof);
(ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and
a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof);
(ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and
a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof);
(ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and
a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof);
(ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and
a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or
(af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and
a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof).

39. The recombinant viral capsid protein of any one of claims 1-38, wherein the antibody or binding portion thereof that binds an extracellular domain of TfR1 comprises:
(i) a HCVR that comprises the amino acid sequence set forth in SEQ ID NO: 2 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 (or a variant thereof);
(vi) a HCVR that comprises the amino acid sequence set forth in SEQ ID NO: 52 (or a variant thereof); and a LCVR that comprises the amino acid sequence set forth in SEQ ID NO: 57 (or a variant thereof);
(vii) a HCVR that comprises the amino acid sequence set forth in SEQ ID NO: 62 (or a variant thereof); and a LCVR that comprises the amino acid sequence set forth in SEQ ID NO: 67 (or a variant thereof);
(viii) a HCVR that comprises the amino acid sequence set forth in SEQ ID NO: 72 (or a variant thereof); and a LCVR that comprises the amino acid sequence set forth in SEQ ID NO: 77 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 (or a variant thereof); and a LCVR that comprises the amino acid sequence set forth in SEQ ID NO: 117 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 (or a variant thereof); and a LCVR that comprises the amino acid sequence set forth in SEQ ID NO: 147 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 (or a variant thereof); and a LCVR that comprises the amino acid sequence set forth in SEQ ID NO: 187 (or a variant thereof);
(xx) a HCVR that comprises the amino acid sequence set forth in SEQ ID NO: 192 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ ID NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 (or a variant thereof); and a LCVR that comprises the amino acid sequence set forth in SEQ ID NO: 307 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

40. The recombinant viral capsid protein of any one of claims 1-38, wherein the antibody or binding portion thereof that binds an extracellular domain of TfR1 comprises:
(i) a HCVR that comprises the amino acid sequence set forth in SEQ ID NO: 2 (or a variant thereof); and a LCVR that comprises the amino acid sequence set forth in SEQ ID NO: 7 (or a variant thereof);
(ii) a HCVR that comprises the amino acid sequence set forth in SEQ ID NO: 42 (or a variant thereof); and a LCVR that comprises the amino acid sequence set forth in SEQ ID NO: 47 (or a variant thereof);
(iii) a HCVR that comprises the amino acid sequence set forth in SEQ ID NO: 122 (or a variant thereof); and a LCVR that comprises the amino acid sequence set forth in SEQ ID NO: 127 (or a variant thereof);
(iv) a HCVR that comprises the amino acid sequence set forth in SEQ ID NO: 132 (or a variant thereof); and a LCVR that comprises the amino acid sequence set forth in SEQ ID NO: 137 (or a variant thereof);
(v) a HCVR that comprises the amino acid sequence set forth in SEQ ID NO: 212 (or a variant thereof); and a LCVR that comprises the amino acid sequence set forth in SEQ ID NO: 217 (or a variant thereof);
(vi) a HCVR that comprises the amino acid sequence set forth in SEQ ID NO: 222 (or a variant thereof); and a LCVR that comprises the amino acid sequence set forth in SEQ ID NO: 227 (or a variant thereof);
(vii) a HCVR that comprises the amino acid sequence set forth in SEQ ID NO: 232 (or a variant thereof); and a LCVR that comprises the amino acid sequence set forth in SEQ ID NO: 237 (or a variant thereof);
(viii) a HCVR that comprises the amino acid sequence set forth in SEQ ID NO: 242 (or a variant thereof); and a LCVR that comprises the amino acid sequence set forth in SEQ ID NO: 247 (or a variant thereof);
(ix) a HCVR that comprises the amino acid sequence set forth in SEQ ID NO: 262 (or a variant thereof); and a LCVR that comprises the amino acid sequence set forth in SEQ ID NO: 267 (or a variant thereof);
(x) a HCVR that comprises the amino acid sequence set forth in SEQ ID NO: 272 (or a variant thereof); and a LCVR that comprises the amino acid sequence set forth in SEQ ID NO: 277 (or a variant thereof);
(xi) a HCVR that comprises the amino acid sequence set forth in SEQ ID NO: 282 (or a variant thereof); and a LCVR that comprises the amino acid sequence set forth in SEQ ID NO: 287 (or a variant thereof); and/or
(xii) a HCVR that comprises the amino acid sequence set forth in SEQ ID NO: 292 (or a variant thereof); and a LCVR that comprises the amino acid sequence set forth in SEQ ID NO: 297 (or a variant thereof).

41. A recombinant viral capsid comprising the recombinant viral capsid protein of any one of claims 1-40.

42. The recombinant viral capsid of claim 41, wherein the viral capsid is a mosaic viral capsid and further comprises a reference viral capsid protein that is at least 95% identical to the recombinant viral capsid protein, and
wherein the reference viral capsid protein lacks all three of (i) the first member of the protein:protein binding pair, (ii) the second member of the protein:protein binding pair, and (iii) the antibody or binding portion thereof.

43. The recombinant viral capsid of claim 42, wherein the mosaic viral capsid comprises the reference viral capsid protein and the recombinant viral capsid protein at a ratio of at least 2:1, 3:1, 4:1, 5:1, 6:1, 7:1. 8:1, 9:1, 10:1, 11:1, 12:1, 13:1, 14:1, 15:1, 16:1, 17:1, 18:1, 19:1, 20:1.

44. The recombinant viral capsid of any one of claims 41-43, further comprising a nucleotide of interest encapsidated within the viral capsid.

45. The recombinant viral capsid of claim 44, wherein the nucleotide of interest is a reporter gene.

46. The recombinant viral capsid of claim 44 or claim 45, wherein the nucleotide of interest encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

47. The recombinant viral capsid of claim 44, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

48. The recombinant viral capsid of any one of claims 44-47, wherein the nucleotide of interest is operably linked to a promoter that is organ-specific, tissue-specific, or cell-specific.

49. The recombinant viral capsid of claim 48, wherein the promoter is brain-specific.

50. The recombinant viral capsid of claim 48 or claim 49, wherein the promoter is neuron-specific, glial cell-specific, astrocyte-specific, oligodendrocyte-specific, microglia-specific and/or central nervous system-specific.

51. The recombinant viral capsid of any one of claims 48-50, wherein the promoter is selected from the group consisting of human glial fibrillary acidic protein (GFAP) promoter, human synapsin 1 (SYN1) promoter, human synapsin 2 (SYN2) promoter, human metallothionein 3 (MT3) promoter, and human proteolipid protein 1 (PLP1) promoter.

52. The recombinant viral capsid of claim 48 or claim 49, wherein the promoter is a neuron-, astrocyte-, or oligodendrocyte-specific, or neuron-, astrocyte-, or oligodendrocyte-preferential promoter.

53. The recombinant viral capsid of claim 52, wherein the promoter is selected from the group consisting of: an NSE promoter, a Synapsin promoter, a MeCP2 promoter, an oligodendrocyte transcription factor 1 (Olig1) promoter, a chondroitin sulfate proteoglycan (Cspg4) promoter, a CNP (2′,3′-Cyclic-nucleotide 3′-phosphodiesterase) promoter, and a GFAP promoter.

54. A pharmaceutical composition comprising (a) the recombinant viral capsid of any one of claims 41-53 and (b) a pharmaceutically acceptable carrier or excipient.

55. A method of delivering a nucleotide of interest across a blood brain barrier in a mammalian subject, the method comprising administering the pharmaceutical composition of claim 54 to the mammal.

56. The method of claim 55, wherein the administering comprises intravenous injection.

57. The method of claim 56, wherein the subject is modified to express the targeting ligand, e.g., from a safe harbor locus.

58. The method of claim 56, wherein the subject is a primate mammal, preferably a human.

59. The method of any one of claims 54-58, wherein the mammalian blood brain barrier cell is a mammalian brain endothelial cell.

60. The method of any one of claims 54-59, wherein endothelial cells in the mammalian blood brain barrier express transferrin receptor protein 1 on the cell surface and (i) the first member of the protein:protein binding pair, (ii) the second member of the protein:protein binding pair, and (iii) the antibody or binding portion thereof together direct the tropism of the viral vector to the endothelial cells in the mammalian blood brain barrier.

61. The method of claim 60, wherein the viral particle is transported across the interior of the endothelial cells of the blood brain barrier for delivery to the brain by transcytosis after binding of the viral particle to the transferrin receptor protein 1 on the endothelial cell surface such that the endothelial cells are not infected by the viral particle.

62. The method of any one of claims 55-61, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

63. The method of claim 62, wherein the therapeutic protein is a soluble protein.

64. The method of claim 62 or 63, wherein the therapeutic protein comprises an antibody or binding portion thereof.

65. The method of claim 64, wherein the administering comprises intravenous or intracerebroventricular injection.

66. The method of claim 62, wherein the nucleotide of interest encodes an shRNA molecule.

67. The method of claims 55-66, wherein the nucleotide of interest is operably linked to a brain-specific promoter and the nucleotide of interest is preferentially expressed in the brain over other organs or tissues.

68. A method of treating a disease in a patient in need thereof comprising:
administering to the patient (a) the viral particle or composition according to any one of claims 1-53 or (b) the pharmaceutical composition of claim 54,
wherein the viral particle comprises a nucleotide of interest encapsidated within the viral capsid, and
wherein the nucleotide of interest encodes a therapeutic moiety selected from the group consisting of: a therapeutic protein, a suicide gene, an antibody or a fragment thereof, a CRISPR/Cas system or a portion(s) thereof, an antisense oligonucleotide, a ribozyme, an RNAi molecule, and a shRNA molecule.

69. The method of claim 68, wherein the therapeutic moiety targets α-synuclein.

70. The method of claim 69, wherein the therapeutic moiety comprises a SNCA shRNA molecule.

71. Use of the viral particle or composition according to any one of claims 1-53 or the pharmaceutical composition of claim 54 for the manufacture of a medicament for the treatment of disease.

72. A viral protein, capsid, genome, particle, and methods of making and using the same, including in the manufacture of a medicament, substantially as herein described.
